(19) United States
(12) Patent Application Publication
(10) Pub. No.: US 2008/0200467 A1
(43) Pub. Date: Aug. 21, 2008

(54) SOLUBLE EPOXIDE HYDROLASE INHIBITORS

(75) Inventors: Dinesh Patel, Fremont, CA (US); Richard D. Gless, Oakland, CA (US); Heather K. Webb Hsu, San Francisco, CA (US); Sampath Kumar Anandan, Fremont, CA (US); Bhaskar R. Aavula, Pleasanton, CA (US)

Correspondence Address: FOLEY & LARDNER LLP, 975 PAGE MILL ROAD, PALO ALTO, CA, 94304, US

(73) Assignee: Arete Therapeutics, Inc.

(21) Appl. No.: 11/934,698

(22) Filed: Nov. 2, 2007

Related U.S. Application Data
(60) Provisional application No. 60/856,408, filed on Nov. 2, 2006.

(51) Int. Cl. A61K 31/5375; C07C 237/00; A61K 31/164; C07D 233/54; A61P 9/00; A61P 3/10; A61P 11/00; A61K 31/4164; A61K 31/445; C07D 295/00

(52) U.S. Cl. 514/237.8; 564/169; 514/621; 544/162; 564/188; 514/623; 546/233; 514/331; 548/338.1; 514/399

(57) ABSTRACT

Disclosed are alpha keto amide and alpha hydroxy amide compounds and compositions that inhibit soluble epoxide hydrolase (sEH), methods for preparing the compounds and compositions, and methods for treating patients with such compounds and compositions. The compounds, compositions, and methods are useful for treating a variety of sEH mediated diseases, including hypertensive, cardiovascular, inflammatory, pulmonary, and diabetic-related diseases.

### CROSS-REFERENCE TO RELATED APPLICATION

This application claims the benefit under 35 U.S.C. § 119(e) of U.S. Provisional Patent Application Ser. No. 60/856,408, filed on Nov. 2, 2006, which is incorporated herein by reference in its entirety.

### BACKGROUND OF THE INVENTION

1. Field of the Invention

This invention relates to the field of pharmaceutical chemistry. Provided herein are alpha keto amide and alpha hydroxylamide compounds that inhibit soluble epoxide hydrolase (sEH), pharmaceutical compositions containing such compounds, methods for preparing the compounds and formulations, and methods for treating patients with such compounds and compositions. The compounds, compositions, and methods are useful for treating a variety of sEH mediated diseases, including hypertensive, cardiovascular, inflammatory, pulmonary, and diabetic-related diseases.

2. State of the Art

The arachidonate cascade is a ubiquitous lipid signaling cascade in which arachidonic acid is liberated from the plasma membrane lipid reserves in response to a variety of extra-cellular and/or intra-cellular signals. The released arachidonic acid is then available to act as a substrate for a variety of oxidative enzymes that convert arachidonic acid to signaling lipids that play critical roles, for example, in inflammation. Disruption of the pathways leading to the lipids remains an important strategy for many commercial drugs used to treat a multitude of inflammatory disorders. For example, non-steroidal anti-inflammatory drugs (NSAIDs) disrupt the conversion of arachidonic acid to prostaglandins by inhibiting cyclooxygenases (COX1 and COX2). New asthma drugs, such as SINGULAIR™ disrupt the conversion of arachidonic acid to leukotrienes by inhibiting lipoxygenase (LOX).

Certain cytochrome P450-dependent enzymes convert arachidonic acid into a series of epoxide derivatives known as epoxyeicosatrienoic acids (EETs). These EETs are particularly prevalent in endothelium (cells that make up arteries and vascular beds), kidney, and lung. In contrast to many of the end products of the prostaglandin and leukotriene pathways, the EETs have a variety of anti-inflammatory and anti-hypertensive properties and are known to be potent vasodilators and mediators of vascular permeability.

While EETs have potent effects in vivo, the epoxide moiety of the EETs is rapidly hydrolyzed into the less active dihydroxyeicosatrienoic acid (DHET) form by an enzyme called soluble epoxide hydrolase (sEH). Inhibition of sEH has been found to significantly reduce blood pressure in hypertensive animals (see, e.g., Yu et al. Circ. Res. 87:992-8 (2000) and Sinal et al. J. Biol. Chem. 275:40504-10 (2000)), to reduce the production of proinflammatory nitric oxide (NO), cytokines, and lipid mediators, and to contribute to inflammatory resolution by enhancing lipoxin A₄ production in vivo (see Schmelzer et al. Proc. Nat'l Acad. Sci. USA 102(28):9772-7 (2005)).

Various small molecule compounds have been found to inhibit sEH and elevate EET levels (Morisseau et al. Annu. Rev. Pharmacol. Toxicol. 45:311-33 (2005)). The availability of more potent compounds capable of inhibiting sEH and its inactivation of EETs would be highly desirable for treating a wide range of disorders that arise from inflammation and hypertension or that are otherwise mediated by sEH.

### SUMMARY OF THE INVENTION

This invention relates to compounds and their pharmaceutical compositions, to their preparation, and to their uses for treating diseases mediated by soluble epoxide hydrolase (sEH). In accordance with one aspect of the invention, provided is a compound or stereoisomer of Formula A or a pharmaceutically acceptable salt of the compound or the stereoisomer:

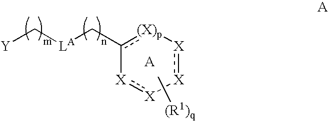

wherein
Y is selected from the group consisting of cycloalkyl, substituted cycloalkyl, phenyl, substituted phenyl, heterocyclyl, substituted heterocyclyl, heteroaryl, and substituted heteroaryl;
L^{A} is a linker of the formula:

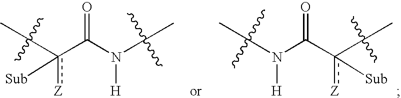

Sub is not present and Z is O if [symbol] connected thereto is a double bond, or Sub is hydrogen or alkyl, and Z is OH if [symbol] connected thereto is a single bond;
each X in ring A is independently selected from the group consisting of N, NH, NR¹, O, CH, CH₂, CHR¹, and CR¹R¹, with the proviso that at least two X's of the A ring are independently CH, CH₂, CHR¹, or CR¹R¹;
p is zero or one;
each R¹ is independently selected from the group consisting of alkyl, substituted alkyl, alkenyl, substituted alkenyl, alkynyl, substituted alkynyl, aryl, substituted aryl, heteroaryl, substituted heteroaryl, cycloalkyl, substituted cycloalkyl, heterocyclic, substituted heterocyclic, halo, hydroxy, nitro, cyano, alkoxy, substituted alkoxy, aryloxy, substituted aryloxy, acyl, aminocarbonyl, carboxy, carboxy ester, amino, substituted amino, acylamino, (carboxyl ester)amino, aminocarbonylamino, aminosulfonyl, substituted sulfonyl, and (substituted sulfonyl)amino;
q is 0, 1, 2, 3, or 4;
[symbol] of the ring A indicates a single or double bond;
m is 0, 1, 2, 3, 4, or 5; and
n is 0, 1, 2, 3, 4, or 5;
provided that the compound or pharmaceutically acceptable salt thereof is not
N-(1-benzylpiperidin-4-yl)-2-hydroxy-3-methyl-2-o-tolylbutanamide;
N-(1-benzylpiperidin-4-yl)-2-(3,5-difluorophenyl)-2-hydroxy-3-methylbutanamide;
N-(1-(4-fluorobenzyl)piperidin-4-yl)-2-hydroxy-3-methyl-2-phenylbutanamide;
N-(1-benzylpiperidin-4-yl)-2-hydroxy-3-methyl-2-(3-(trifluoromethyl)phenyl)butanamide;
N-(1-benzylpiperidin-4-yl)-2-hydroxy-3-methyl-2-m-tolylbutanamide;
N-(1-benzylpiperidin-4-yl)-3-ethyl-2-hydroxy-2-phenylpentanamide;
2-hydroxy-2-phenyl-N-(4-(piperidin-1-yl)phenyl)acetamide;
N-(2,4-dimethylphenyl)-2-oxo-2-phenylacetamide;
N1,N2-bis(2-(2-(4-nitrophenylamino)-2-oxoacetyl)phenyl)oxalamide;
2-(2,6-diphenoxyphenyl)-N-(6-methyl-2,4-bis(methylthio)pyridin-3-yl)-2-oxoacetamide;
2-(4-fluorophenyl)-N-(6-methyl-2,4-bis(methylthio)pyridin-3-yl)-2-oxoacetamide;
2-(4-bromophenyl)-N-(6-methyl-2,4-bis(methylthio)pyridin-3-yl)-2-oxoacetamide;
4-(2-(4-(dimethylamino)phenyl)-2-oxoacetamido)benzenesulfonic acid;
2-(2-acetamido-5-bromophenyl)-N-(4-ethoxyphenyl)-2-oxoacetamide;
N-(3-methoxy-4-(oxazol-5-yl)phenyl)-2-oxo-2-phenylacetamide;
2-(2-acetamidophenyl)-N-(4-ethylphenyl)-2-oxoacetamide;
2-(2-acetamidophenyl)-N-(3,5-dichlorophenyl)-2-oxoacetamide;
4-(2-(2-acetamidophenyl)-2-oxoacetamido)-N-phenethylbenzamide;
3-(2-(2-acetamidophenyl)-2-oxoacetamido)-N-(2,4,6-trifluorobenzyl)benzamide;
3-(2-(2-acetamidophenyl)-2-oxoacetamido)-N-(4-chlorophenethyl)benzamide;
2-oxo-N-(4-(4-oxo-4,5,6,7-tetrahydro-1H-pyrrolo[3,2-c]pyridin-2-yl)pyridin-2-yl)-2-phenylacetamide;
2-(2-acetamidophenyl)-N-(3,5-dimethylphenyl)-2-oxoacetamide;
2-(2-acetamidophenyl)-N-(4-(ethylthio)phenyl)-2-oxoacetamide;
2-(4-ethoxydibenzo[b,d]furan-1-yl)-2-oxo-N-(pyridin-4-yl)acetamide;
N-(2-(1-naphthamido)ethyl)-4-(2-(4-nitrophenyl)-2-oxoacetamido)piperidine-4-carboxamide;
2-oxo-N,2-di-p-tolylacetamide; or
3-methyl-2-(2-oxo-2-(p-tolylamino)acetyl)benzenesulfonic acid.

In Formula A, no two adjacent X groups may be both oxygen.

In one embodiment of the invention, provided is a compound or stereoisomer of Formula I or a pharmaceutically acceptable salt of the compound or the stereoisomer:

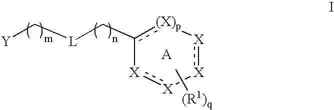

wherein
Y is selected from the group consisting of cycloalkyl, substituted cycloalkyl, phenyl, substituted phenyl, heterocyclyl, substituted heterocyclyl, heteroaryl, and substituted heteroaryl;
L is a linker of the formula:

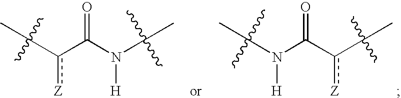

Z is O if [symbol] connected thereto is a double bond or Z is OH if [symbol] connected thereto is a single bond;
each X in ring A is independently selected from the group consisting of N, NH, NR¹, O, CH, CH₂, CHR¹, and CR¹R¹, with the proviso that at least two X's of the A ring are independently CH, CH₂, CHR¹, or CR¹R¹;
p is zero or one;
each R¹ is independently selected from the group consisting of alkyl, substituted alkyl, alkenyl, substituted alkenyl, alkynyl, substituted alkynyl, aryl, substituted aryl, heteroaryl, substituted heteroaryl, cycloalkyl, substituted cycloalkyl, heterocyclic, substituted heterocyclic, halo, hydroxy, nitro, cyano, alkoxy, substituted alkoxy, aryloxy, substituted aryloxy, acyl, aminocarbonyl, carboxy, carboxy ester, amino, substituted amino, acylamino, (carboxyl ester)amino, aminocarbonylamino, aminosulfonyl, substituted sulfonyl, and (substituted sulfonyl)amino;
q is 0, 1, 2, 3, or 4;
[symbol] of the ring A indicates a single or double bond;
m is 0, 1, 2, 3, 4, or 5; and
n is 0, 1, 2, 3, 4, or 5;
provided that the compound or pharmaceutically acceptable salt thereof is not 2-hydroxy-2-phenyl-N-(4-(piperidin-1-yl)phenyl)acetamide;
N-(2,4-dimethylphenyl)-2-oxo-2-phenylacetamide;
N1,N2-bis(2-(2-(4-nitrophenylamino)-2-oxoacetyl)phenyl)oxalamide;
2-(2,6-diphenoxyphenyl)-N-(6-methyl-2,4-bis(methylthio)pyridin-3-yl)-2-oxoacetamide;
2-(4-fluorophenyl)-N-(6-methyl-2,4-bis(methylthio)pyridin-3-yl)-2-oxoacetamide;
2-(4-bromophenyl)-N-(6-methyl-2,4-bis(methylthio)pyridin-3-yl)-2-oxoacetamide;
4-(2-(4-(dimethylamino)phenyl)-2-oxoacetamido)benzenesulfonic acid;
2-(2-acetamido-5-bromophenyl)-N-(4-ethoxyphenyl)-2-oxoacetamide;
N-(3-methoxy-4-(oxazol-5-yl)phenyl)-2-oxo-2-phenylacetamide;
2-(2-acetamidophenyl)-N-(4-ethylphenyl)-2-oxoacetamide;
2-(2-acetamidophenyl)-N-(3,5-dichlorophenyl)-2-oxoacetamide;
4-(2-(2-acetamidophenyl)-2-oxoacetamido)-N-phenethylbenzamide;
3-(2-(2-acetamidophenyl)-2-oxoacetamido)-N-(2,4,6-trifluorobenzyl)benzamide;
3-(2-(2-acetamidophenyl)-2-oxoacetamido)-N-(4-chlorophenethyl)benzamide;
2-oxo-N-(4-(4-oxo-4,5,6,7-tetrahydro-1H-pyrrolo[3,2-c]pyridin-2-yl)pyridin-2-yl)-2-phenylacetamide;
2-(2-acetamidophenyl)-N-(3,5-dimethylphenyl)-2-oxoacetamide;
2-(2-acetamidophenyl)-N-(4-(ethylthio)phenyl)-2-oxoacetamide;
2-(4-ethoxydibenzo[b,d]furan-1-yl)-2-oxo-N-(pyridin-4-yl)acetamide;
N-(2-(1-naphthamido)ethyl)-4-(2-(4-nitrophenyl)-2-oxoacetamido)piperidine-4-carboxamide;
2-oxo-N,2-di-p-tolylacetamide; or
3-methyl-2-(2-oxo-2-(p-tolylamino)acetyl)benzenesulfonic acid.

In Formula I, no two adjacent X groups may be both oxygen.

In another embodiment, provided is a compound or stereoisomer of Formula II or a pharmaceutically acceptable salt of the compound or the stereoisomer:

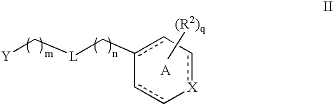

wherein
Y is selected from the group consisting of cyclohexyl, substituted cyclohexyl, adamantyl, substituted adamantyl, phenyl, substituted phenyl, heterocyclyl, substituted heterocyclyl, heteroaryl, and substituted heteroaryl;
L is a linker of the formula:

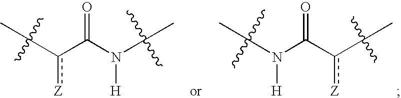

Z is O if [symbol] connected thereto is a double bond or Z is OH if [symbol] connected thereto is a single bond;
X in ring A is selected from the group consisting of N, NH, NR², O, CH, CH₂, CHR², and CR²R²;
each R² is independently selected from the group consisting of alkyl, substituted alkyl, alkenyl, substituted alkenyl, alkynyl, substituted alkynyl, aryl, substituted aryl, heteroaryl, substituted heteroaryl, cycloalkyl, substituted cycloalkyl, heterocyclic, substituted heterocyclic, halo, hydroxy, nitro, cyano, alkoxy, substituted alkoxy, aryloxy, substituted aryloxy, acyl, aminocarbonyl, carboxy, carboxy ester, amino, substituted amino, acylamino, (carboxyl ester)amino, aminocarbonylamino, aminosulfonyl, substituted sulfonyl, and (substituted sulfonyl)amino;
q is 0, 1, 2, 3, or 4;
[symbol] of the ring A indicates a single or double bond;
m is 0, 1, 2, 3, 4, or 5; and
n is 0, 1, 2, 3, 4, or 5;
provided that the compound or pharmaceutically acceptable salt thereof is not 2-hydroxy-2-phenyl-N-(4-(piperidin-1-yl)phenyl)acetamide;
N-(2,4-dimethylphenyl)-2-oxo-2-phenylacetamide;
N1,N2-bis(2-(2-(4-nitrophenylamino)-2-oxoacetyl)phenyl)oxalamide;
2-(2,6-diphenoxyphenyl)-N-(6-methyl-2,4-bis(methylthio)pyridin-3-yl)-2-oxoacetamide;
2-(4-fluorophenyl)-N-(6-methyl-2,4-bis(methylthio)pyridin-3-yl)-2-oxoacetamide;
2-(4-bromophenyl)-N-(6-methyl-2,4-bis(methylthio)pyridin-3-yl)-2-oxoacetamide;
4-(2-(4-(dimethylamino)phenyl)-2-oxoacetamido)benzenesulfonic acid;
2-(2-acetamido-5-bromophenyl)-N-(4-ethoxyphenyl)-2-oxoacetamide;
N-(3-methoxy-4-(oxazol-5-yl)phenyl)-2-oxo-2-phenylacetamide;
2-(2-acetamidophenyl)-N-(4-ethylphenyl)-2-oxoacetamide;
2-(2-acetamidophenyl)-N-(3,5-dichlorophenyl)-2-oxoacetamide;
4-(2-(2-acetamidophenyl)-2-oxoacetamido)-N-phenethylbenzamide;
3-(2-(2-acetamidophenyl)-2-oxoacetamido)-N-(2,4,6-trifluorobenzyl)benzamide;
3-(2-(2-acetamidophenyl)-2-oxoacetamido)-N-(4-chlorophenethyl)benzamide;
2-oxo-N-(4-(4-oxo-4,5,6,7-tetrahydro-1H-pyrrolo[3,2-c]pyridin-2-yl)pyridin-2-yl)-2-phenylacetamide;
2-(2-acetamidophenyl)-N-(3,5-dimethylphenyl)-2-oxoacetamide;
2-(2-acetamidophenyl)-N-(4-(ethylthio)phenyl)-2-oxoacetamide;
2-(4-ethoxydibenzo[b,d]furan-1-yl)-2-oxo-N-(pyridin-4-yl)acetamide;
N-(2-(1-naphthamido)ethyl)-4-(2-(4-nitrophenyl)-2-oxoacetamido)piperidine-4-carboxamide;
2-oxo-N,2-di-p-tolylacetamide; or
3-methyl-2-(2-oxo-2-(p-tolylamino)acetyl)benzenesulfonic acid.

In another embodiment, provided is a compound or stereoisomer of Formula III or a pharmaceutically acceptable salt of the compound or the stereoisomer:

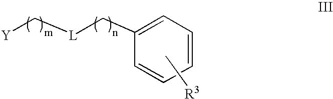

wherein
Y is selected from the group consisting of cyclohexyl, substituted cyclohexyl, adamantyl, substituted adamantyl, phenyl, substituted phenyl, heterocyclyl, substituted heterocyclyl, heteroaryl, and substituted heteroaryl;
L is a linker of the formula:

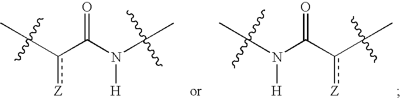

Z is O if [symbol] connected thereto is a double bond or Z is OH if [symbol] connected thereto is a single bond;
R³ is selected from the group consisting of alkyl, substituted alkyl, alkenyl, substituted alkenyl, alkynyl, substituted alkynyl, aryl, substituted aryl, heteroaryl, substituted heteroaryl, cycloalkyl, substituted cycloalkyl, heterocyclic, substituted heterocyclic, halo, hydroxy, nitro, cyano, alkoxy, substituted alkoxy, aryloxy, substituted aryloxy, acyl, aminocarbonyl, carboxy, carboxy ester, amino, substituted amino, acylamino, (carboxyl ester)amino, aminocarbonylamino, aminosulfonyl, substituted sulfonyl, and (substituted sulfonyl)amino;
m is 0, 1, or 2; and
n is 0, 1, or 2;
provided that the compound or pharmaceutically acceptable salt thereof is not
2-hydroxy-2-phenyl-N-(4-(piperidin-1-yl)phenyl)acetamide;
N-(2,4-dimethylphenyl)-2-oxo-2-phenylacetamide; N1,N2-bis(2-(2-(4-nitrophenylamino)-2-oxoacetyl)phenyl)oxalamide;
2-(2,6-diphenoxyphenyl)-N-(6-methyl-2,4-bis(methylthio)pyridin-3-yl)-2-oxoacetamide;
2-(4-fluorophenyl)-N-(6-methyl-2,4-bis(methylthio)pyridin-3-yl)-2-oxoacetamide;
2-(4-bromophenyl)-N-(6-methyl-2,4-bis(methylthio)pyridin-3-yl)-2-oxoacetamide;
4-(2-(4-(dimethylamino)phenyl)-2-oxoacetamido)benzenesulfonic acid;
2-(2-acetamido-5-bromophenyl)-N-(4-ethoxyphenyl)-2-oxoacetamide;
N-(3-methoxy-4-(oxazol-5-yl)phenyl)-2-oxo-2-phenylacetamide;
2-(2-acetamidophenyl)-N-(4-ethylphenyl)-2-oxoacetamide;
2-(2-acetamidophenyl)-N-(3,5-dichlorophenyl)-2-oxoacetamide;
4-(2-(2-acetamidophenyl)-2-oxoacetamido)-N-phenethylbenzamide;
3-(2-(2-acetamidophenyl)-2-oxoacetamido)-N-(2,4,6-trifluorobenzyl)benzamide;
3-(2-(2-acetamidophenyl)-2-oxoacetamido)-N-(4-chlorophenethyl)benzamide;
2-oxo-N-(4-(4-oxo-4,5,6,7-tetrahydro-1H-pyrrolo[3,2-c]pyridin-2-yl)pyridin-2-yl)-2-phenylacetamide;
2-(2-acetamidophenyl)-N-(3,5-dimethylphenyl)-2-oxoacetamide;
2-(2-acetamidophenyl)-N-(4-(ethylthio)phenyl)-2-oxoacetamide;
2-(4-ethoxydibenzo[b,d]furan-1-yl)-2-oxo-N-(pyridin-4-yl)acetamide;
N-(2-(1-naphthamido)ethyl)-4-(2-(4-nitrophenyl)-2-oxoacetamido)piperidine-4-carboxamide;
2-oxo-N,2-di-p-tolylacetamide; or
3-methyl-2-(2-oxo-2-(p-tolylamino)acetyl)benzenesulfonic acid.

In another embodiment, provided is a compound or stereoisomer of Formula IV or a pharmaceutically acceptable salt of the compound or the stereoisomer:

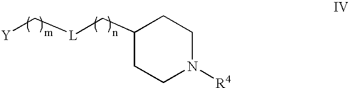

wherein
Y is selected from the group consisting of cyclohexyl, substituted cyclohexyl, adamantyl, substituted adamantyl, phenyl, substituted phenyl, heterocyclyl, substituted heterocyclyl, heteroaryl, and substituted heteroaryl;
L is a linker of the formula:

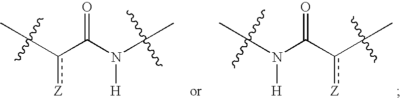

Z is O if [symbol] connected thereto is a double bond or Z is OH if [symbol] connected thereto is a single bond;
R⁴ is selected from the group consisting of alkyl, substituted alkyl, alkenyl, substituted alkenyl, alkynyl, substituted alkynyl, aryl, substituted aryl, heteroaryl, substituted heteroaryl, cycloalkyl, substituted cycloalkyl, heterocyclic, substituted heterocyclic, cyano, acyl, aminocarbonyl, aminosulfonyl, substituted sulfonyl, (substituted sulfonyl)amino, and carboxy ester;
m is 0, 1, or 2; and
n is 0, 1, or 2.

In accordance with another embodiment of the invention, provided is a compound or stereoisomer of Formula V or a pharmaceutically acceptable salt of the compound or the stereoisomer:

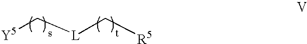

wherein
Y⁵ is selected from the group consisting of cycloalkyl, substituted cycloalkyl, phenyl, substituted phenyl, heterocyclyl, substituted heterocyclyl, heteroaryl, and substituted heteroaryl;
L is a linker of the formula:

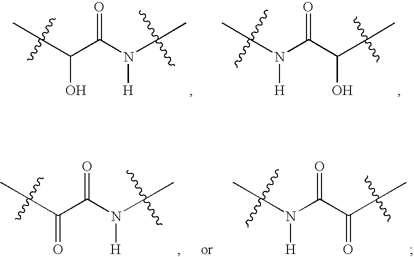

R⁵ is selected from the group consisting of alkyl, alkenyl, alkynyl, hydroxy, nitro, cyano, alkoxy, substituted alkoxy, aryloxy, substituted aryloxy, acyl, aminocarbonyl, carboxy, carboxy ester, amino, substituted amino, acylamino, (carboxyl ester)amino, aminocarbonylamino, aminosulfonyl, substituted sulfonyl, (substituted sulfonyl)amino, phenyl, substituted phenyl, heteroaryl, and substituted heteroaryl;
s is 0, 1, 2, 3, 4, or 5; and
t is 0, 1, 2, 3, 4, 5, 6, 7, 8, 9, 10, 11, 12, or 13.

In accordance with another aspect of the invention, provided is a method for treating a soluble epoxide hydrolase mediated disease, said method comprising administering to a patient a pharmaceutical composition comprising a pharmaceutically acceptable carrier and a therapeutically effective amount of a compound or stereoisomer of Formula I or a pharmaceutically acceptable salt of the compound or the stereoisomer:

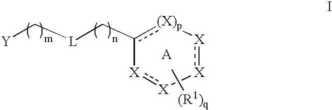

wherein
Y is selected from the group consisting of cycloalkyl, substituted cycloalkyl, phenyl, substituted phenyl, heterocyclyl, substituted heterocyclyl, heteroaryl, and substituted heteroaryl;
L is a linker of the formula:

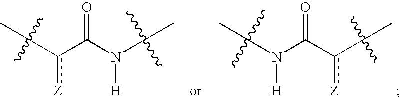

Z is O if [symbol] connected thereto is a double bond or Z is OH if [symbol] connected thereto is a single bond;
each X in ring A is independently selected from the group consisting of N, NH, NR¹, O, CH, CH₂, CHR¹, and CR¹R¹, with the proviso that at least two X's of the A ring are independently CH, CH₂, CHR¹, or CR¹R¹;
p is zero or one;
each R¹ is independently selected from the group consisting of alkyl, substituted alkyl, alkenyl, substituted alkenyl, alkynyl, substituted alkynyl, aryl, substituted aryl, heteroaryl, substituted heteroaryl, cycloalkyl, substituted cycloalkyl, heterocyclic, substituted heterocyclic, halo, hydroxy, nitro, cyano, alkoxy, substituted alkoxy, aryloxy, substituted aryloxy, acyl, aminocarbonyl, carboxy, carboxy ester, amino, substituted amino, acylamino, (carboxyl ester)amino, aminocarbonylamino, aminosulfonyl, substituted sulfonyl, and (substituted sulfonyl)amino;
q is 0, 1, 2, 3, or 4;
[symbol] of the ring A indicates a single or double bond;
m is 0, 1, 2, 3, 4, or 5; and
n is 0, 1, 2, 3, 4, or 5.

In accordance with yet another aspect of the invention, provided is a method for treating a soluble epoxide hydrolase mediated disease, said method comprising administering to a patient a pharmaceutical composition comprising a pharmaceutically acceptable carrier and a therapeutically effective amount of a compound or stereoisomer of Formula V or a pharmaceutically acceptable salt of the compound or the stereoisomer:

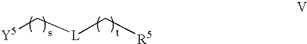

wherein
Y⁵ is selected from the group consisting of cycloalkyl, substituted cycloalkyl, phenyl, substituted phenyl, heterocyclyl, substituted heterocyclyl, heteroaryl, and substituted heteroaryl;
L is a linker of the formula:

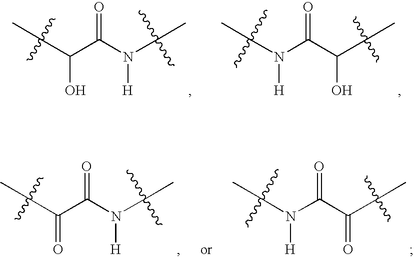

R⁵ is selected from the group consisting of alkyl, alkenyl, alkynyl, hydroxy, nitro, cyano, alkoxy, substituted alkoxy, aryloxy, substituted aryloxy, acyl, aminocarbonyl, carboxy, carboxy ester, amino, substituted amino, acylamino, (carboxyl ester)amino, aminocarbonylamino, aminosulfonyl, substituted sulfonyl, (substituted sulfonyl)amino, phenyl, substituted phenyl, heteroaryl, and substituted heteroaryl;
s is 0, 1, 2, 3, 4, or 5; and
t is 0, 1, 2, 3, 4, 5, 6, 7, 8, 9, 10, 11, 12, or 13.

These and other embodiments of the invention are further described in the Detailed Description that follows.

### DETAILED DESCRIPTION OF THE INVENTION

### Definitions

As used herein, the following definitions shall apply unless otherwise indicated.

“cis-Epoxyeicosatrienoic acids” (“EETs”) are biomediators synthesized by cytochrome P450 epoxygenases.

“Epoxide hydrolases” (“EH;” EC 3.3.2.3) are enzymes in the alpha/beta hydrolase fold family that add water to 3 membered cyclic ethers termed epoxides.

“Soluble epoxide hydrolase” (“sEH”) is an enzyme which in endothelial, smooth muscle and other cell types converts EETs to dihydroxy derivatives called dihydroxyeicosatrienoic acids (“DHETs”). The cloning and sequence of the murine sEH is set forth in Grant et al., J. Biol. Chem. 268(23):17628-17633 (1993). The cloning, sequence, and accession numbers of the human sEH sequence are set forth in Beetham et al., Arch. Biochem. Biophys. 305(1):197-201 (1993). The amino acid sequence of human sEH is also set forth as SEQ ID NO:2 of U.S. Pat. No. 5,445,956; the nucleic acid sequence encoding the human sEH is set forth as nucleotides 42-1703 of SEQ ID NO: 1 of that patent. The evolution and nomenclature of the gene is discussed in Beetham et al., DNA Cell Biol. 14(1):61-71 (1995). Soluble epoxide hydrolase represents a single highly conserved gene product with over 90% homology between rodent and human (Arand et al., FEBS Lett., 338:251-256 (1994)).

“Chronic Obstructive Pulmonary Disease” or “COPD” is also sometimes known as “chronic obstructive airway disease”, “chronic obstructive lung disease”, and “chronic airways disease.” COPD is generally defined as a disorder characterized by reduced maximal expiratory flow and slow forced emptying of the lungs. COPD is considered to encompass two related conditions, emphysema and chronic bronchitis. COPD can be diagnosed by the general practitioner using art recognized techniques, such as the patient's forced vital capacity (“FVC”), the maximum volume of air that can be forcibly expelled after a maximal inhalation. In the offices of general practitioners, the FVC is typically approximated by a 6 second maximal exhalation through a spirometer. The definition, diagnosis and treatment of COPD, emphysema, and chronic bronchitis are well known in the art and discussed in detail by, for example, Honig and Ingram, in Harrison's Principles of Internal Medicine, (Fauci et al., Eds), 14th Ed., 1998, McGraw-Hill, New York, pp. 1451-1460 (hereafter, “Harrison's Principles of Internal Medicine”). As the names imply, “obstructive pulmonary disease” and “obstructive lung disease” refer to obstructive diseases, as opposed to restrictive diseases. These diseases particularly include COPD, bronchial asthma, and small airway disease.

“Emphysema” is a disease of the lungs characterized by permanent destructive enlargement of the airspaces distal to the terminal bronchioles without obvious fibrosis.

“Chronic bronchitis” is a disease of the lungs characterized by chronic bronchial secretions which last for most days of a month, for three months, a year, for two years, etc.

“Small airway disease” refers to diseases where airflow obstruction is due, solely or predominantly to involvement of the small airways. These are defined as airways less than 2 mm in diameter and correspond to small cartilaginous bronchi, terminal bronchioles, and respiratory bronchioles. Small airway disease (SAD) represents luminal obstruction by inflammatory and fibrotic changes that increase airway resistance. The obstruction may be transient or permanent.

“Interstitial lung diseases (ILDs)” are restrictive lung diseases involving the alveolar walls, perialveolar tissues, and contiguous supporting structures. As discussed on the website of the American Lung Association, the tissue between the air sacs of the lung is the interstitium, and this is the tissue affected by fibrosis in the disease. Persons with such restrictive lung disease have difficulty breathing in because of the stiffness of the lung tissue but, in contrast to persons with obstructive lung disease, have no difficulty breathing out. The definition, diagnosis and treatment of interstitial lung diseases are well known in the art and discussed in detail by, for example, Reynolds, H. Y., in Harrison's Principles of Internal Medicine, supra, at pp. 1460-1466. Reynolds notes that, while ILDs have various initiating events, the immunopathological responses of lung tissue are limited and the ILDs therefore have common features.

“Idiopathic pulmonary fibrosis,” or “IPF,” is considered the prototype ILD. Although it is idiopathic in that the cause is not known, Reynolds, supra, notes that the term refers to a well defined clinical entity.

“Bronchoalveolar lavage,” or “BAL,” is a test which permits removal and examination of cells from the lower respiratory tract and is used in humans as a diagnostic procedure for pulmonary disorders such as IPF. In human patients, it is usually performed during bronchoscopy.

“Diabetic neuropathy” refers to acute and chronic peripheral nerve dysfunction resulting from diabetes.

“Diabetic nephropathy” refers to renal diseases resulting from diabetes.

“Alkyl” refers to monovalent saturated aliphatic hydrocarbyl groups having from 1 to 10 carbon atoms and preferably 1 to 6 carbon atoms. This term includes, by way of example, linear and branched hydrocarbyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃)(CH₃CH₂)CH—), t-butyl ((CH₃)₃C—), n-pentyl (CH₃CH₂CH₂CH₂CH₂—), and neopentyl ((CH₃)₃CCH₂—).

“Alkenyl” refers to straight or branched hydrocarbyl groups having from 2 to 6 carbon atoms and preferably 2 to 4 carbon atoms and having at least 1 and preferably from 1 to 2 sites of vinyl (>C═C<) unsaturation. Such groups are exemplified, for example, by vinyl, allyl, and but-3-en-1-yl. Included within this term are the cis and trans isomers or mixtures of these isomers.

“Alkynyl” refers to straight or branched monovalent hydrocarbyl groups having from 2 to 6 carbon atoms and preferably 2 to 3 carbon atoms and having at least 1 and preferably from 1 to 2 sites of acetylenic (—C≡C—) unsaturation. Examples of such alkynyl groups include acetylenyl (—C≡CH), and propargyl (—CH₂C≡CH).

“Substituted alkyl” refers to an alkyl group having from 1 to 5, preferably 1 to 3, or more preferably 1 to 2 substituents selected from the group consisting of alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are defined herein.

“Substituted alkenyl” refers to alkenyl groups having from 1 to 3 substituents, and preferably 1 to 2 substituents, selected from the group consisting of alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are defined herein and with the proviso that any hydroxy or thiol substitution is not attached to a vinyl (unsaturated) carbon atom.

“Substituted alkynyl” refers to alkynyl groups having from 1 to 3 substituents, and preferably 1 to 2 substituents, selected from the group consisting of alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are defined herein and with the proviso that any hydroxy or thiol substitution is not attached to an acetylenic carbon atom.

“Alkoxy” refers to the group —O-alkyl wherein alkyl is defined herein. Alkoxy includes, by way of example, methoxy, ethoxy, n-propoxy, isopropoxy, n-butoxy, t-butoxy, sec-butoxy, and n-pentoxy.

“Substituted alkoxy” refers to the group —O-(substituted alkyl) wherein substituted alkyl is defined herein.

“Acyl” refers to the groups H—C(O)—, alkyl-C(O)—, substituted alkyl-C(O)—, alkenyl-C(O)—, substituted alkenyl-C(O)—, alkynyl-C(O)—, substituted alkynyl-C(O)—, cycloalkyl-C(O)—, substituted cycloalkyl-C(O)—, cycloalkenyl-C(O)—, substituted cycloalkenyl-C(O)—, aryl-C(O)—, substituted aryl-C(O)—, heteroaryl-C(O)—, substituted heteroaryl-C(O)—, heterocyclic-C(O)—, and substituted heterocyclic-C(O)—, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. Acyl includes the “acetyl” group CH₃C(O)—.

“Acylamino” refers to the groups —NRC(O)alkyl, —NRC(O)substituted alkyl, —NRC(O)cycloalkyl, —NRC(O)substituted cycloalkyl, —NRC(O)cycloalkenyl, —NRC(O)substituted cycloalkenyl, —NRC(O)alkenyl, —NRC(O)substituted alkenyl, —NRC(O)alkynyl, —NRC(O)substituted alkynyl, —NRC(O)aryl, —NRC(O)substituted aryl, —NRC(O)heteroaryl, —NRC(O)substituted heteroaryl, —NRC(O)heterocyclic, and —NRC(O)substituted heterocyclic wherein R is hydrogen or alkyl and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Acyloxy” refers to the groups alkyl-C(O)O—, substituted alkyl-C(O)O—, alkenyl-C(O)O—, substituted alkenyl-C(O)O—, alkynyl-C(O)O—, substituted alkynyl-C(O)O—, aryl-C(O)O—, substituted aryl-C(O)O—, cycloalkyl-C(O)O—, substituted cycloalkyl-C(O)O—, cycloalkenyl-C(O)O—, substituted cycloalkenyl-C(O)O—, heteroaryl-C(O)O—, substituted heteroaryl-C(O)O—, heterocyclic-C(O)O—, and substituted heterocyclic-C(O)O— wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Amino” refers to the group —NH₂.

“Substituted amino” refers to the group —NR′R″ where R′ and R″ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, substituted heterocyclic, —SO₂-alkyl, —SO₂-substituted alkyl, —SO₂-alkenyl, —SO₂-substituted alkenyl, —SO₂-cycloalkyl, —SO₂-substituted cycloalkyl, —SO₂-cycloalkenyl, —SO₂-substituted cycloalkenyl, —SO₂-aryl, —SO₂-substituted aryl, —SO₂-heteroaryl, —SO₂-substituted heteroaryl, —SO₂-heterocyclic, and —SO₂-substituted heterocyclic and wherein R′ and R″ are optionally joined, together with the nitrogen bound thereto to form a heterocyclic or substituted heterocyclic group, provided that R′ and R″ are both not hydrogen, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. When R′ is hydrogen and R″ is alkyl, the substituted amino group is sometimes referred to herein as alkylamino. When R′ and R″ are alkyl, the substituted amino group is sometimes referred to herein as dialkylamino. When referring to a monosubstituted amino, it is meant that either R′ or R″ is hydrogen but not both. When referring to a disubstituted amino, it is meant that neither R′ nor R″ are hydrogen.

“Aminocarbonyl” refers to the group —C(O)NR¹⁰R¹¹ where R¹⁰ and R¹¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R¹⁰ and R¹¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminothiocarbonyl” refers to the group —C(S)NR¹⁰R¹¹ where R¹⁰ and R¹¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R¹⁰ and R¹¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminocarbonylamino” refers to the group —NRC(O)NR¹⁰R¹¹ where R is hydrogen or alkyl and R¹⁰ and R¹¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R¹⁰ and R¹¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminothiocarbonylamino” refers to the group —NRC(S)NR¹⁰R¹¹ where R is hydrogen or alkyl and R¹⁰ and R¹¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R¹⁰ and R¹¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminocarbonyloxy” refers to the group —O—C(O)NR¹⁰R¹¹ where R¹⁰ and R¹¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R¹⁰ and R¹¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminosulfonyl” refers to the group —SO₂NR¹⁰R¹¹ where R¹⁰ and R¹¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R¹⁰ and R¹¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminosulfonyloxy” refers to the group —O—SO₂NR¹⁰R¹¹ where R¹⁰ and R¹¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R¹⁰ and R¹¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminosulfonylamino” refers to the group —NR—SO₂NR¹⁰R¹¹ where R is hydrogen or alkyl and R¹⁰ and R¹¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R¹⁰ and R¹¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Amidino” refers to the group —C(═NR¹²)NR¹⁰R¹¹ where R¹⁰, R¹¹, and R¹² are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R¹⁰ and R¹¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aryl” or “Ar” refers to a monovalent aromatic carbocyclic group of from 6 to 14 carbon atoms having a single ring (e.g., phenyl) or multiple condensed rings (e.g., naphthyl or anthryl) which condensed rings may or may not be aromatic (e.g., 2-benzoxazolinone, 2H-1,4-benzoxazin-3(4H)-one-7-yl, and the like) provided that the point of attachment is at an aromatic carbon atom. Preferred aryl groups include phenyl and naphthyl.

“Substituted aryl” refers to aryl groups which are substituted with 1 to 5, preferably 1 to 3, or more preferably 1 to 2 substituents selected from the group consisting of alkyl, substituted alkyl, alkenyl, substituted alkenyl, alkynyl, substituted alkynyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are defined herein.

“Aryloxy” refers to the group —O-aryl, where aryl is as defined herein, that includes, by way of example, phenoxy and naphthoxy.

“Substituted aryloxy” refers to the group —O-(substituted aryl) where substituted aryl is as defined herein.

“Arylthio” refers to the group —S-aryl, where aryl is as defined herein.

“Substituted arylthio” refers to the group —S-(substituted aryl), where substituted aryl is as defined herein.

“Carbonyl” refers to the divalent group —C(O)— which is equivalent to —C(═O)—.

“Carboxy” or “carboxyl” refers to —COOH or salts thereof.

“Carboxyl ester” or “carboxy ester” refers to the groups —C(O)O-alkyl, —C(O)O-substituted alkyl, —C(O)O-alkenyl, —C(O)O-substituted alkenyl, —C(O)O-alkynyl, —C(O)O-substituted alkynyl, —C(O)O-aryl, —C(O)O-substituted aryl, —C(O)O-cycloalkyl, —C(O)O-substituted cycloalkyl, —C(O)O-cycloalkenyl, —C(O)O-substituted cycloalkenyl, —C(O)O-heteroaryl, —C(O)O-substituted heteroaryl, —C(O)O-heterocyclic, and —C(O)O-substituted heterocyclic wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“(Carboxyl ester)amino” refers to the group —NR—C(O)O-alkyl, —NR—C(O)O— substituted alkyl, —NR—C(O)O-alkenyl, —NR—C(O)O-substituted alkenyl, —NR—C(O)O-alkynyl, —NR—C(O)O-substituted alkynyl, —NR—C(O)O-aryl, —NR—C(O)O-substituted aryl, —NR—C(O)O-cycloalkyl, —NR—C(O)O-substituted cycloalkyl, —NR—C(O)O-cycloalkenyl, —NR—C(O)O-substituted cycloalkenyl, —NR—C(O)O-heteroaryl, —NR—C(O)O-substituted heteroaryl, —NR—C(O)O-heterocyclic, and —NR—C(O)O-substituted heterocyclic wherein R is alkyl or hydrogen, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“(Carboxyl ester)oxy” refers to the group —O—C(O)O-alkyl, —O—C(O)O-substituted alkyl, —O—C(O)O-alkenyl, —O—C(O)O-substituted alkenyl, —O—C(O)O-alkynyl, —O—C(O)O-substituted alkynyl, —O—C(O)O-aryl, —O—C(O)O-substituted aryl, —O—C(O)O-cycloalkyl, —O—C(O)O-substituted cycloalkyl, —O—C(O)O-cycloalkenyl, —O—C(O)O-substituted cycloalkenyl, —O—C(O)O-heteroaryl, —O—C(O)O-substituted heteroaryl, —O—C(O)O-heterocyclic, and —O—C(O)O-substituted heterocyclic wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Cyano” refers to the group —CN.

“Cycloalkyl” refers to cyclic alkyl groups of from 3 to 10 carbon atoms having single or multiple cyclic rings including fused, bridged, and spiro ring systems. One or more of the rings can be aryl, heteroaryl, or heterocyclic provided that the point of attachment is through the non-aromatic, non-heterocyclic ring carbocyclic ring. Examples of suitable cycloalkyl groups include, for instance, adamantyl, cyclopropyl, cyclobutyl, cyclopentyl, and cyclooctyl. Other examples of cycloalkyl groups include bicycle[2,2,2,]octanyl, norbornyl, and spiro groups such as spiro[4.5]dec-8-yl:

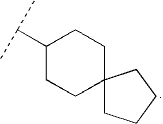

“Cycloalkenyl” refers to non-aromatic cyclic alkyl groups of from 3 to 10 carbon atoms having single or multiple cyclic rings and having at least one >C═C<ring unsaturation and preferably from 1 to 2 sites of >C═C<ring unsaturation.

“Substituted cycloalkyl” and “substituted cycloalkenyl” refers to a cycloalkyl or cycloalkenyl group having from 1 to 5 or preferably 1 to 3 substituents selected from the group consisting of oxo, thione, alkyl, substituted alkyl, alkenyl, substituted alkenyl, alkynyl, substituted alkynyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are defined herein.

“Cycloalkyloxy” refers to —O-cycloalkyl.

“Substituted cycloalkyloxy” refers to —O-(substituted cycloalkyl).

“Cycloalkylthio” refers to —S-cycloalkyl.

“Substituted cycloalkylthio” refers to —S-(substituted cycloalkyl).

“Cycloalkenyloxy” refers to —O-cycloalkenyl.

“Substituted cycloalkenyloxy” refers to —O-(substituted cycloalkenyl).

“Cycloalkenylthio” refers to —S-cycloalkenyl.

“Substituted cycloalkenylthio” refers to —S-(substituted cycloalkenyl).

“Guanidino” refers to the group —NHC(═NH)NH₂.

“Substituted guanidino” refers to —NR¹³C(═NR¹³)N(R¹³)₂ where each R¹³ is independently selected from the group consisting of hydrogen, alkyl, substituted alkyl, aryl, substituted aryl, heteroaryl, substituted heteroaryl, heterocyclic, and substituted heterocyclic and two R¹³ groups attached to a common guanidino nitrogen atom are optionally joined together with the nitrogen bound thereto to form a heterocyclic or substituted heterocyclic group,
provided that at least one R¹³ is not hydrogen, and wherein said substituents are as defined herein.

“Halo” or “halogen” refers to fluoro, chloro, bromo and iodo and preferably is fluoro or chloro.

“Haloalkyl” refers to alkyl groups substituted with 1 to 5, 1 to 3, or 1 to 2 halo groups, wherein alkyl and halo are as defined herein.

“Haloalkoxy” refers to alkoxy groups substituted with 1 to 5, 1 to 3, or 1 to 2 halo groups, wherein alkoxy and halo are as defined herein.

“Haloalkylthio” refers to alkylthio groups substituted with 1 to 5, 1 to 3, or 1 to 2 halo groups, wherein alkylthio and halo are as defined herein.

“Hydroxy” or “hydroxyl” refers to the group —OH.

“Heteroaryl” refers to an aromatic group of from 1 to 10 carbon atoms and 1 to 4 heteroatoms selected from the group consisting of oxygen, nitrogen and sulfur within the ring. Such heteroaryl groups can have a single ring (e.g., pyridinyl or furyl) or multiple condensed rings (e.g., indolizinyl or benzothienyl) wherein the condensed rings may or may not be aromatic and/or contain a heteroatom provided that the point of attachment is through an atom of the aromatic heteroaryl group. In one embodiment, the nitrogen and/or the sulfur ring atom(s) of the heteroaryl group are optionally oxidized to provide for the N-oxide (N→O), sulfinyl, or sulfonyl moieties. Preferred heteroaryls include pyridinyl, pyrrolyl, indolyl, thiophenyl, and furanyl.

“Substituted heteroaryl” refers to heteroaryl groups that are substituted with from 1 to 5, preferably 1 to 3, or more preferably 1 to 2 substituents selected from the group consisting of the same group of substituents defined for substituted aryl.

“Heteroaryloxy” refers to —O-heteroaryl.

“Substituted heteroaryloxy” refers to the group —O-(substituted heteroaryl).

“Heteroarylthio” refers to the group —S-heteroaryl.

“Substituted heteroarylthio” refers to the group —S-(substituted heteroaryl).

“Heterocycle” or “heterocyclic” or “heterocycloalkyl” or “heterocyclyl” refers to a saturated or partially saturated, but not aromatic, group having from 1 to 10 ring carbon atoms and from 1 to 4 ring heteroatoms selected from the group consisting of nitrogen, sulfur, or oxygen. Heterocycle encompasses single ring or multiple condensed rings, including fused bridged and spiro ring systems. In fused ring systems, one or more the rings can be cycloalkyl, aryl, or heteroaryl provided that the point of attachment is through the non-aromatic ring. In one embodiment, the nitrogen and/or sulfur atom(s) of the heterocyclic group are optionally oxidized to provide for the N-oxide, sulfinyl, or sulfonyl moieties.

“Substituted heterocyclic” or “substituted heterocycloalkyl” or “substituted heterocyclyl” refers to heterocyclyl groups that are substituted with from 1 to 5 or preferably 1 to 3 of the same substituents as defined for substituted cycloalkyl.

“Heterocyclyloxy” refers to the group —O-heterocyclyl.

“Substituted heterocyclyloxy” refers to the group —O-(substituted heterocyclyl).

“Heterocyclylthio” refers to the group —S-heterocyclyl.

“Substituted heterocyclylthio” refers to the group —S-(substituted heterocyclyl).

Examples of heterocycle and heteroaryls include, but are not limited to, azetidine, pyrrole, imidazole, pyrazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo[b]thiophene, thiazole, thiazolidine, thiophene, benzo[b]thiophene, morpholinyl, thiomorpholinyl (also referred to as thiamorpholinyl), 1,1-dioxothiomorpholinyl, piperidinyl, pyrrolidine, and tetrahydrofuranyl.

“Nitro” refers to the group —NO₂.

“Oxo” refers to the atom (═O) or (—O—).

“Spiro ring systems” refers to bicyclic ring systems that have a single ring carbon atom common to both rings.

“Sulfonyl” refers to the divalent group —S(O)₂—.

“Substituted sulfonyl” refers to the group —SO₂-alkyl, —SO₂-substituted alkyl, —SO₂-alkenyl, —SO₂-substituted alkenyl, —SO₂-cycloalkyl, —SO₂-substituted cycloalkyl, —SO₂-cycloalkenyl, —SO₂-substituted cycloalkenyl, —SO₂-aryl, —SO₂-substituted aryl, —SO₂-heteroaryl, —SO₂-substituted heteroaryl, —SO₂-heterocyclic, —SO₂-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. Substituted sulfonyl includes groups such as methyl-SO₂—, phenyl-SO₂—, and 4-methylphenyl-SO₂—. The term “alkylsulfonyl” refers to —SO₂-alkyl. The term “haloalkylsulfonyl” refers to —SO₂-haloalkyl where haloalkyl is defined herein. The term “(substituted sulfonyl)amino” refers to —NH(substituted sulfonyl) wherein substituted sulfonyl is as defined herein.

“Sulfonyloxy” refers to the group —OSO₂-alkyl, —OSO₂-substituted alkyl, —OSO₂-alkenyl, —OSO₂-substituted alkenyl, —OSO₂-cycloalkyl, —OSO₂-substituted cycloalkyl, —OSO₂-cycloalkenyl, —OSO₂-substituted cycloalkenyl, —OSO₂-aryl, —OSO₂-substituted aryl, —OSO₂-heteroaryl, —OSO₂-substituted heteroaryl, —OSO₂-heterocyclic, —OSO₂-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Thioacyl” refers to the groups H—C(S)—, alkyl-C(S)—, substituted alkyl-C(S)—, alkenyl-C(S)—, substituted alkenyl-C(S)—, alkynyl-C(S)—, substituted alkynyl-C(S)—, cycloalkyl-C(S)—, substituted cycloalkyl-C(S)—, cycloalkenyl-C(S)—, substituted cycloalkenyl-C(S)—, aryl-C(S)—, substituted aryl-C(S)—, heteroaryl-C(S)—, substituted heteroaryl-C(S)—, heterocyclic-C(S)—, and substituted heterocyclic-C(S)—, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Thiol” refers to the group —SH.

“Thiocarbonyl” refers to the divalent group —C(S)— which is equivalent to —C(═S)—.

“Thione” refers to the atom (═S).

“Alkylthio” refers to the group —S-alkyl wherein alkyl is as defined herein.

“Substituted alkylthio” refers to the group —S-(substituted alkyl) wherein substituted alkyl is as defined herein.

“Stereoisomer” or “stereoisomers” refer to compounds that differ in the chirality of one or more stereocenters. Stereoisomers include enantiomers and diastereomers.

“Tautomer” refer to alternate forms of a compound that differ in the position of a proton, such as enol-keto and imine-enamine tautomers, or the tautomeric forms of heteroaryl groups containing a ring atom attached to both a ring —NH— moiety and a ring ═N— moiety such as pyrazoles, imidazoles, benzimidazoles, triazoles, and tetrazoles.

“Patient” refers to mammals and includes humans and non-human mammals.

“Pharmaceutically acceptable salt” refers to pharmaceutically acceptable salts of a compound, which salts are derived from a variety of organic and inorganic counter ions well known in the art and include, by way of example only, sodium, potassium, calcium, magnesium, ammonium, and tetraalkylammonium; and when the molecule contains a basic functionality, salts of organic or inorganic acids, such as hydrochloride, hydrobromide, tartrate, mesylate, acetate, maleate, and oxalate.

“Treating” or “treatment” of a disease in a patient refers to (1) preventing the disease from occurring in a patient that is predisposed or does not yet display symptoms of the disease; (2) inhibiting the disease or arresting its development; or (3) ameliorating or causing regression of the disease.

Unless indicated otherwise, the nomenclature of substituents that are not explicitly defined herein are arrived at by naming the terminal portion of the functionality followed by the adjacent functionality toward the point of attachment. For example, the substituent “arylalkyloxycarbonyl” refers to the group (aryl)-(alkyl)-O—C(O)—.

It is understood that in all substituted groups defined above, polymers arrived at by defining substituents with further substituents to themselves (e.g., substituted aryl having a substituted aryl group as a substituent which is itself substituted with a substituted aryl group, which is further substituted by a substituted aryl group etc) are not intended for inclusion herein. In such cases, the maximum number of such substitutions is three. For example, serial substitutions of substituted aryl groups with two other substituted aryl groups are limited to -substituted aryl-(substituted aryl)-substituted aryl.

Similarly, it is understood that the above definitions are not intended to include impermissible substitution patterns (e.g., methyl substituted with 5 fluoro groups). Such impermissible substitution patterns are well known to the skilled artisan.

This invention relates to compounds and their pharmaceutical compositions, to their preparation, and to their uses for treating diseases mediated by soluble epoxide hydrolase (sEH). In accordance with one aspect of the invention, provided is a compound or stereoisomer of Formula A or a pharmaceutically acceptable salt of the compound or the stereoisomer:

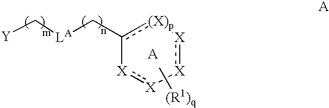

wherein
Y is selected from the group consisting of cycloalkyl, substituted cycloalkyl, phenyl, substituted phenyl, heterocyclyl, substituted heterocyclyl, heteroaryl, and substituted heteroaryl;
L^{A} is a linker of the formula:

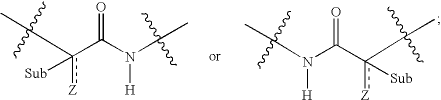

Sub is not present and Z is O if [symbol] connected thereto is a double bond, or Sub is hydrogen or alkyl, and Z is OH if [symbol] connected thereto is a single bond;
each X in ring A is independently selected from the group consisting of N, NH, NR¹, O, CH, CH₂, CHR¹, and CR¹R¹, with the proviso that at least two X's of the A ring are independently CH, CH₂, CHR¹, or CR¹R¹;
p is zero or one;
each R¹ is independently selected from the group consisting of alkyl, substituted alkyl, alkenyl, substituted alkenyl, alkynyl, substituted alkynyl, aryl, substituted aryl, heteroaryl, substituted heteroaryl, cycloalkyl, substituted cycloalkyl, heterocyclic, substituted heterocyclic, halo, hydroxy, nitro, cyano, alkoxy, substituted alkoxy, aryloxy, substituted aryloxy, acyl, aminocarbonyl, carboxy, carboxy ester, amino, substituted amino, acylamino, (carboxyl ester)amino, aminocarbonylamino, aminosulfonyl, substituted sulfonyl, and (substituted sulfonyl)amino;
q is 0, 1, 2, 3, or 4;
[symbol] of the ring A indicates a single or double bond;
m is 0, 1, 2, 3, 4, or 5; and
n is 0, 1, 2, 3, 4, or 5;
provided that the compound or pharmaceutically acceptable salt thereof is not
N-(1-benzylpiperidin-4-yl)-2-hydroxy-3-methyl-2-o-tolylbutanamide;
N-(1-benzylpiperidin-4-yl)-2-(3,5-difluorophenyl)-2-hydroxy-3-methylbutanamide;
N-(1-(4-fluorobenzyl)piperidin-4-yl)-2-hydroxy-3-methyl-2-phenylbutanamide;
N-(1-benzylpiperidin-4-yl)-2-hydroxy-3-methyl-2-(3-(trifluoromethyl)phenyl)butanamide;
N-(1-benzylpiperidin-4-yl)-2-hydroxy-3-methyl-2-m-tolylbutanamide;
N-(1-benzylpiperidin-4-yl)-3-ethyl-2-hydroxy-2-phenylpentanamide;
2-hydroxy-2-phenyl-N-(4-(piperidin-1-yl)phenyl)acetamide;
N-(2,4-dimethylphenyl)-2-oxo-2-phenylacetamide;
N1,N2-bis(2-(2-(4-nitrophenylamino)-2-oxoacetyl)phenyl)oxalamide;
2-(2,6-diphenoxyphenyl)-N-(6-methyl-2,4-bis(methylthio)pyridin-3-yl)-2-oxoacetamide;
2-(4-fluorophenyl)-N-(6-methyl-2,4-bis(methylthio)pyridin-3-yl)-2-oxoacetamide;
2-(4-bromophenyl)-N-(6-methyl-2,4-bis(methylthio)pyridin-3-yl)-2-oxoacetamide;
4-(2-(4-(dimethylamino)phenyl)-2-oxoacetamido)benzenesulfonic acid;
2-(2-acetamido-5-bromophenyl)-N-(4-ethoxyphenyl)-2-oxoacetamide;
N-(3-methoxy-4-(oxazol-5-yl)phenyl)-2-oxo-2-phenylacetamide;
2-(2-acetamidophenyl)-N-(4-ethylphenyl)-2-oxoacetamide;
2-(2-acetamidophenyl)-N-(3,5-dichlorophenyl)-2-oxoacetamide;
4-(2-(2-acetamidophenyl)-2-oxoacetamido)-N-phenethylbenzamide;
3-(2-(2-acetamidophenyl)-2-oxoacetamido)-N-(2,4,6-trifluorobenzyl)benzamide;
3-(2-(2-acetamidophenyl)-2-oxoacetamido)-N-(4-chlorophenethyl)benzamide;
2-oxo-N-(4-(4-oxo-4,5,6,7-tetrahydro-1H-pyrrolo[3,2-c]pyridin-2-yl)pyridin-2-yl)-2-phenylacetamide;
2-(2-acetamidophenyl)-N-(3,5-dimethylphenyl)-2-oxoacetamide;
2-(2-acetamidophenyl)-N-(4-(ethylthio)phenyl)-2-oxoacetamide;
2-(4-ethoxydibenzo[b,d]furan-1-yl)-2-oxo-N-(pyridin-4-yl)acetamide;
N-(2-(1-naphthamido)ethyl)-4-(2-(4-nitrophenyl)-2-oxoacetamido)piperidine-4-carboxamide;
2-oxo-N,2-di-p-tolylacetamide; or
3-methyl-2-(2-oxo-2-(p-tolylamino)acetyl)benzenesulfonic acid wherein these compounds correspond to the following respective structures:

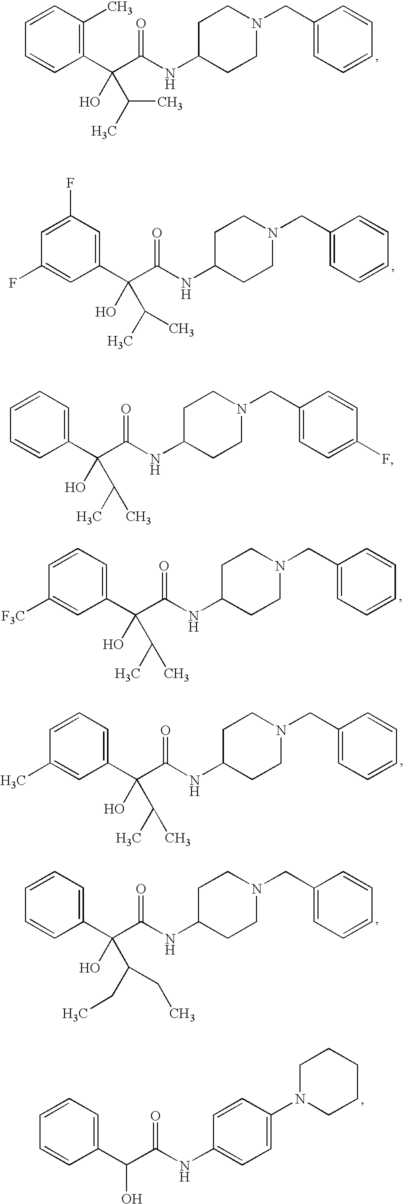

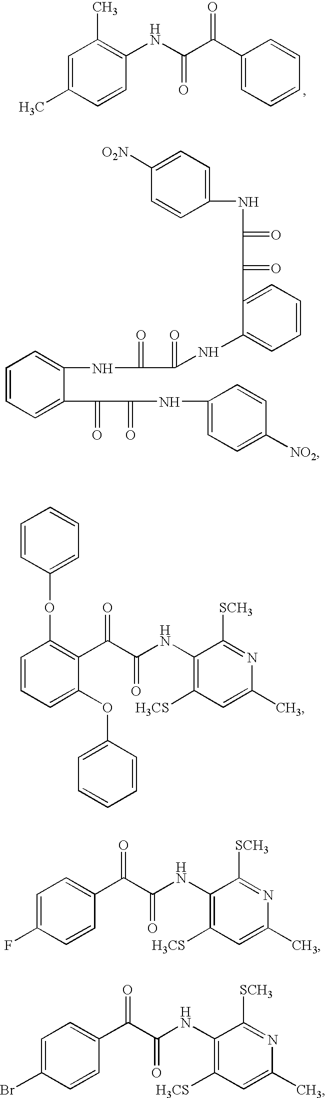

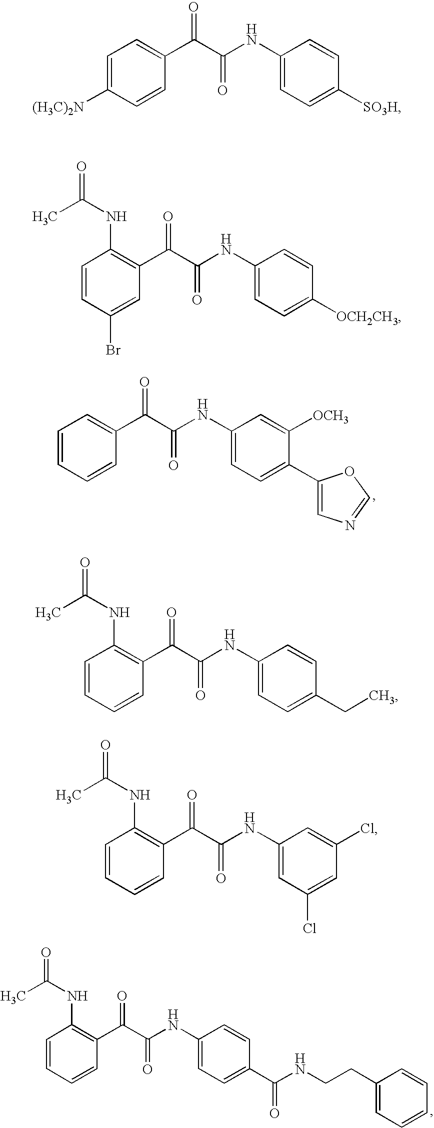

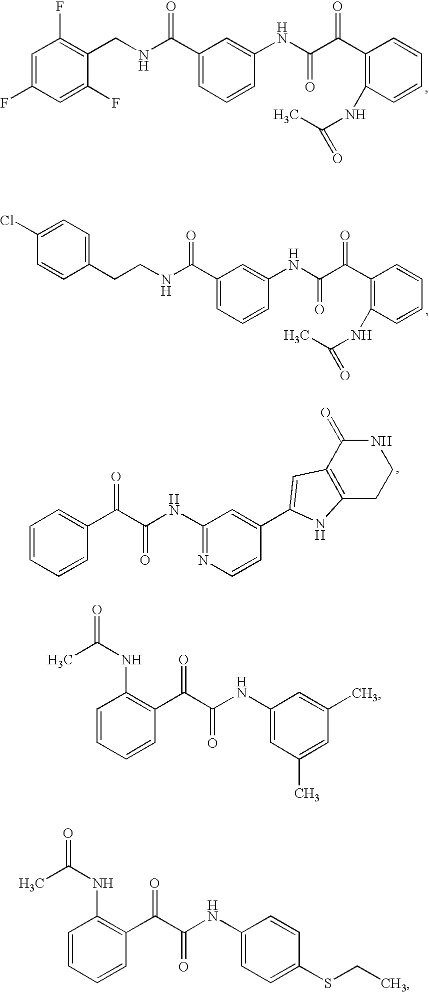

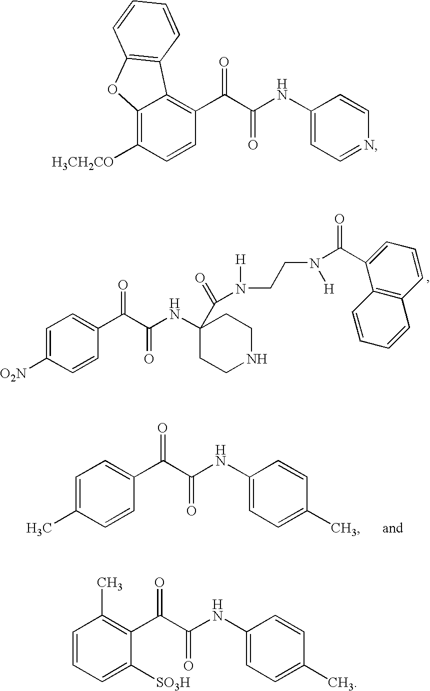

In Formula A, no two adjacent X groups may be both oxygen.

The present invention relates to compounds and their pharmaceutical compositions, to their preparation, and to their uses for treating diseases mediated by soluble epoxide hydrolase (sEH). In accordance with one aspect of the invention, provided is a compound or stereoisomer of Formula I or a pharmaceutically acceptable salt of the compound or the stereoisomer:

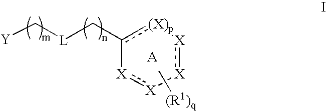

wherein
Y is selected from the group consisting of cycloalkyl, substituted cycloalkyl, phenyl, substituted phenyl, heterocyclyl, substituted heterocyclyl, heteroaryl, and substituted heteroaryl;
L is a linker of the formula:

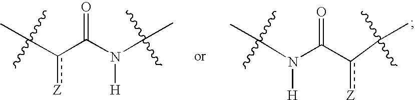

Z is O if [symbol] connected thereto is a double bond or Z is OH if [symbol] connected thereto is a single bond;
each X in ring A is independently selected from the group consisting of N, NH, NR¹, O, CH, CH₂, CHR¹, and CR¹R¹, with the proviso that at least two X's of the A ring are independently CH, CH₂, CHR¹, or CR¹R¹;
p is zero or one;
each R¹ is independently selected from the group consisting of alkyl, substituted alkyl, alkenyl, substituted alkenyl, alkynyl, substituted alkynyl, aryl, substituted aryl, heteroaryl, substituted heteroaryl, cycloalkyl, substituted cycloalkyl, heterocyclic, substituted heterocyclic, halo, hydroxy, nitro, cyano, alkoxy, substituted alkoxy, aryloxy, substituted aryloxy, acyl, aminocarbonyl, carboxy, carboxy ester, amino, substituted amino, acylamino, (carboxyl ester)amino, aminocarbonylamino, aminosulfonyl, substituted sulfonyl, and (substituted sulfonyl)amino;
q is 0, 1, 2, 3, or 4;
[symbol] of the ring A indicates a single or double bond;
m is 0, 1, 2, 3, 4, or 5; and
n is 0, 1, 2, 3, 4, or 5;
provided that the compound or pharmaceutically acceptable salt thereof is not
2-hydroxy-2-phenyl-N-(4-(piperidin-1-yl)phenyl)acetamide;
N-(2,4-dimethylphenyl)-2-oxo-2-phenylacetamide;
N1,N2-bis(2-(2-(4-nitrophenylamino)-2-oxoacetyl)phenyl)oxalamide;
2-(2,6-diphenoxyphenyl)-N-(6-methyl-2,4-bis(methylthio)pyridin-3-yl)-2-oxoacetamide;
2-(4-fluorophenyl)-N-(6-methyl-2,4-bis(methylthio)pyridin-3-yl)-2-oxoacetamide;
2-(4-bromophenyl)-N-(6-methyl-2,4-bis(methylthio)pyridin-3-yl)-2-oxoacetamide;
4-(2-(4-(dimethylamino)phenyl)-2-oxoacetamido)benzenesulfonic acid;
2-(2-acetamido-5-bromophenyl)-N-(4-ethoxyphenyl)-2-oxoacetamide;
N-(3-methoxy-4-(oxazol-5-yl)phenyl)-2-oxo-2-phenylacetamide;
2-(2-acetamidophenyl)-N-(4-ethylphenyl)-2-oxoacetamide;
2-(2-acetamidophenyl)-N-(3,5-dichlorophenyl)-2-oxoacetamide;
4-(2-(2-acetamidophenyl)-2-oxoacetamido)-N-phenethylbenzamide;
3-(2-(2-acetamidophenyl)-2-oxoacetamido)-N-(2,4,6-trifluorobenzyl)benzamide;
3-(2-(2-acetamidophenyl)-2-oxoacetamido)-N-(4-chlorophenethyl)benzamide;
2-oxo-N-(4-(4-oxo-4,5,6,7-tetrahydro-1H-pyrrolo[3,2-c]pyridin-2-yl)pyridin-2-yl)-2-phenylacetamide;
2-(2-acetamidophenyl)-N-(3,5-dimethylphenyl)-2-oxoacetamide;
2-(2-acetamidophenyl)-N-(4-(ethylthio)phenyl)-2-oxoacetamide;
2-(4-ethoxydibenzo[b,d]furan-1-yl)-2-oxo-N-(pyridin-4-yl)acetamide;
N-(2-(1-naphthamido)ethyl)-4-(2-(4-nitrophenyl)-2-oxoacetamido)piperidine-4-carboxamide;
2-oxo-N,2-di-p-tolylacetamide; or
3-methyl-2-(2-oxo-2-(p-tolylamino)acetyl)benzenesulfonic acid wherein these compounds correspond to the following respective structures:

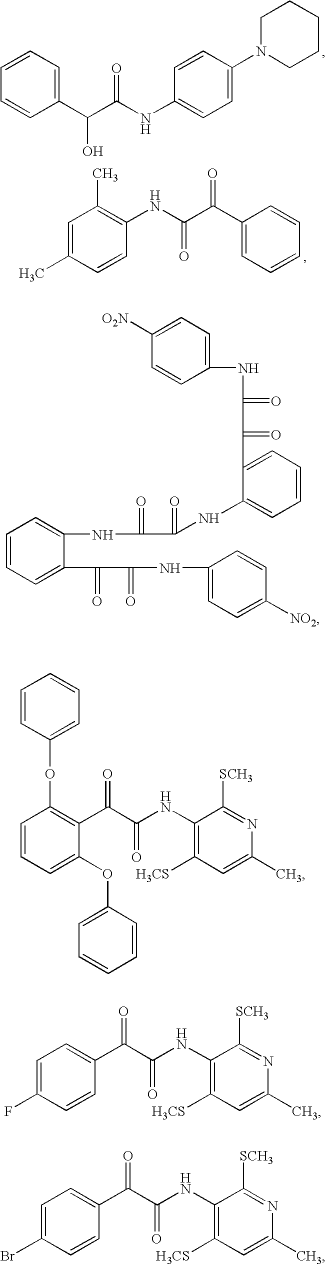

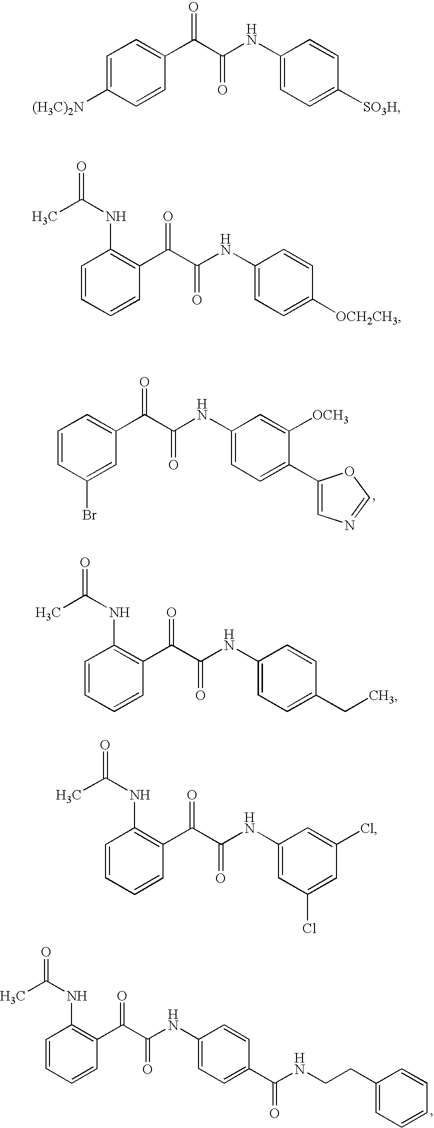

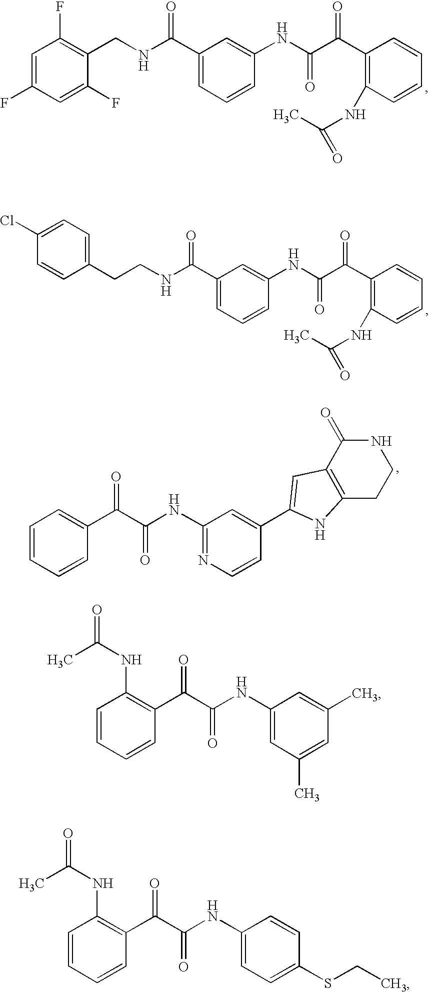

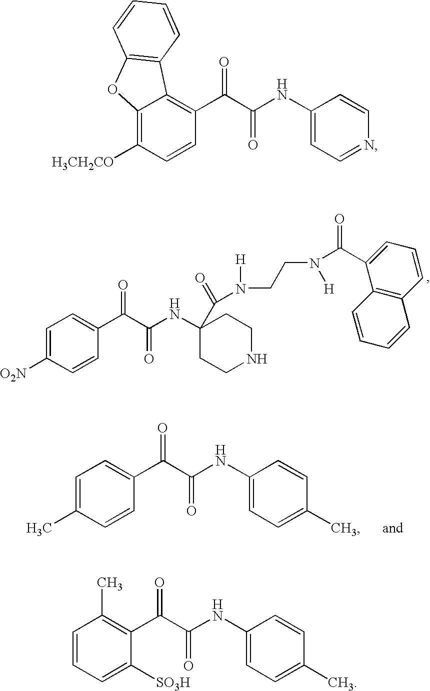

In Formula I, no two adjacent X groups may be both oxygen.

In some embodiments of Formula (A) or Formula (I), Y is cycloalkyl. In some aspects, Y is cyclohexyl. In some aspects, Y is selected from the group consisting of

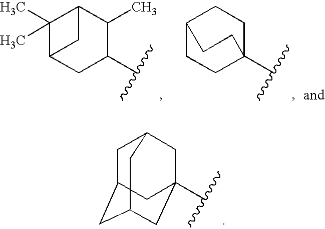

In some aspects, Y is spiro[4.5]dec-8-yl:

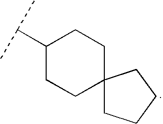

In some embodiments of Formula (A) or Formula (I), Y is C₆₋₁₀ heterocycloalkyl.

In some aspects, Y is quinuclidin-1-yl having the structure

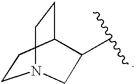

In some embodiments of Formula (A) or Formula (I), Y is phenyl or substituted phenyl.

In some embodiments of Formula (A) or Formula (I), at least four X's of the ring A are independently CH, CH₂, CHR¹, or CR¹R¹.

In some embodiments of Formula (A) or Formula (I), each [symbol] of the ring A is a double bond.

In some embodiments of Formula (A) or Formula (I), each [symbol] of the ring A is a single bond.

In some embodiments of Formula (A) or Formula (I), the ring A is selected from the group consisting of phenyl, pyridinyl, cyclohexyl, and piperidinyl. In some aspects, the ring A is selected from the group consisting of phenyl, piperidinyl, and cyclohexyl.

In some embodiments of Formula (A) or Formula (I), each R¹ is independently selected from the group consisting of alkoxy, substituted alkoxy, aminocarbonyl, haloalkyl, heterocyclic, substituted sulfonyl, acyl, carboxy, carboxyl ester, amino, substituted amino, acylamino, (carboxyl ester)amino, aminosulfonyl, and (substituted sulfonyl)amino.

In some embodiments of Formula (A) or Formula (I), q is 1. In some aspects, when q is 1, R¹ is in the 3-position or 4-position. In some aspects, when q is 1, R¹ is selected from the group consisting of alkoxy, substituted alkoxy, acyl, carboxy, carboxyl ester, aminocarbonyl, amino, substituted amino, acylamino, (carboxyl ester)amino, aminocarbonylamino, aminosulfonyl, substituted sulfonyl, (substituted sulfonyl)amino, haloalkyl, and heterocyclic.

In some embodiments of Formula (A) or Formula (I), Z is O and [symbol] connected thereto is a double bond. In some embodiments of Formula (I), Z is OH and [symbol] connected thereto is a single bond.

In some embodiments of Formula (A) or Formula (I), m is 0, 1, or 2. In some embodiments of Formula (I), n is 0, 1, or 2.

In some embodiments, this invention provides a compound of formula (Ia), or a stereoisomer, or salts thereof:

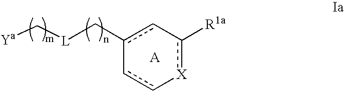

wherein
Y^{a} is C₆₋₁₀ cycloalkyl, substituted C₅₋₁₀ cycloalkyl, or

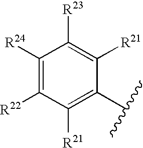

wherein each R²¹ is independently hydrogen or fluoro;
R²², R²¹, and R²⁴ are independently selected from the group consisting of hydrogen, halo, alkyl, acyl, acyloxy, carboxyl ester, acylamino, aminocarbonyl, aminocarbonylamino, aminocarbonyloxy, aminosulfonylamino, (carboxyl ester)amino, aminosulfonyl, (substituted sulfonyl)amino, haloalkyl, haloalkoxy, haloalkylthio, cyano, and alkylsulfonyl;
L is a linker of the formula:

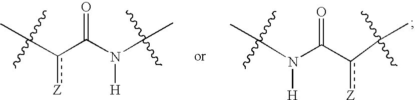

Z is O if [symbol] connected thereto is a double bond or Z is OH if [symbol] connected thereto is a single bond;
X in ring A is selected from the group consisting of N, NH, NR¹, O, CH, CH₂, CHR¹, and CR¹R¹;
each R¹ is independently selected from the group consisting of alkyl, substituted alkyl, alkenyl, substituted alkenyl, alkynyl, substituted alkynyl, aryl, substituted aryl, heteroaryl, substituted heteroaryl, cycloalkyl, substituted cycloalkyl, heterocyclic, substituted heterocyclic, halo, hydroxy, nitro, cyano, alkoxy, substituted alkoxy, aryloxy, substituted aryloxy, acyl, aminocarbonyl, carboxy, carboxy ester, amino, substituted amino, acylamino, (carboxyl ester)amino, aminocarbonylamino, aminosulfonyl, substituted sulfonyl, and (substituted sulfonyl)amino;
R^{1a} is hydrogen or R¹;
[symbol] of the ring A indicates a single or double bond;
m is 0, 1, 2, 3, 4, or 5; and
n is 0, 1, 2, 3, 4, or 5;
provided that the compound is not

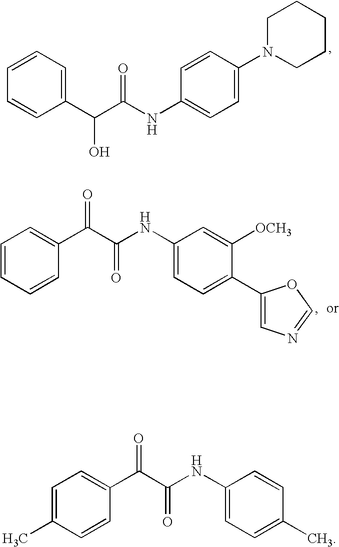

In some embodiments, Y^{a} is C₅₋₁₀ cycloalkyl. In some embodiments, Y^{a} is cyclohexyl. In some embodiments, Y^{a} is adamantyl.

In some embodiments, Y^{a} is

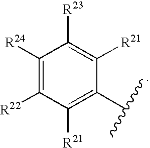

In some embodiments, at least one R²¹ is hydrogen. In some embodiments, both R²¹ are hydrogen.

In some embodiments, at least one of R²², R²³, and R²⁴ is selected from the group consisting of halo, haloalkyl, and halomethoxy. In some embodiments, R²², R²³, and both R are hydrogen, and R²⁴ is selected from the group consisting of fluoro, trifluoromethyl, and trifluoromethoxy. In some embodiments, R²³, R²⁴, and both R²¹ are hydrogen, and R²² is selected from the group consisting of fluoro, trifluoromethyl, and trifluoromethoxy.

In some embodiments, [symbol] of the ring A indicates a single bond, R^{1a} is hydrogen and X is selected from the group consisting of O, NH, NR¹, CH₂, and CHR¹. In some embodiments, X is NR¹ or CHR¹, and R¹ is selected from the group consisting of acyl, aminocarbonyl, substituted sulfonyl. In some embodiments, R¹ is selected from the group consisting of —C(O)-alkyl, —C(O)-substituted alkyl, —C(O)-phenyl, —C(O)-substituted phenyl, —SO₂-alkyl, —SO₂-substituted alkyl, —SO₂-phenyl, —SO₂-substituted phenyl. In some embodiments, the substituted phenyl is selected from the group consisting of tolyl, trifluoromethylphenyl, trifluoromethoxyphenyl, fluorophenyl, and chlorophenyl. In some embodiments, X is CHR¹ and R¹ is alkoxy or substituted alkoxy, preferably R¹ is methoxy.

In some embodiments, [symbol] of the ring A indicates a double bond, and X is N, CH, or CR¹. In some embodiments, X is CR¹, and R^{1a} is hydrogen. In some embodiments, X is CH and R^{1a} is R¹. In some embodiments, R¹ is selected from the group consisting of alkyl, substituted alkyl, alkoxy, substituted alkoxy, and heteroaryl. In some embodiments, R¹ is imidazolyl. In some embodiments, R¹ is methyl, methoxy, trifluoromethyl, trifluoromethoxy, phenoxy, substituted phenoxy, and benzyloxy.

In some embodiments, R¹ is

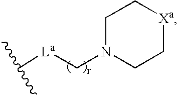

wherein La is —O—, —C(O)—, —C(O)—NH—, r is 0, 1, 2, or 3, and X^{a} is selected from the group consisting of O, S, SO, SO₂, CH₂ and NH.

In some embodiments, L is:

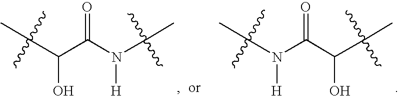

In some embodiments, L is:

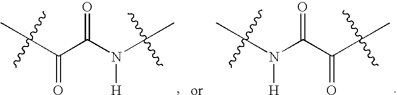

The present invention also relates to compounds, stereoisomers, salts, and their pharmaceutical compositions, to their preparation, and to their uses for treating diseases mediated by soluble epoxide hydrolase (sEH), wherein the compounds, stereoisomers, and pharmaceutically acceptable salts of the compound or the stereoisomer have Formula II:

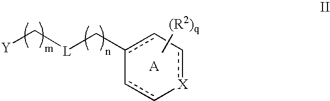

wherein
Y is selected from the group consisting of cyclohexyl, substituted cyclohexyl, adamantyl, substituted adamantyl, phenyl, substituted phenyl, heterocyclyl, substituted heterocyclyl, heteroaryl, and substituted heteroaryl;
L is a linker of the formula:

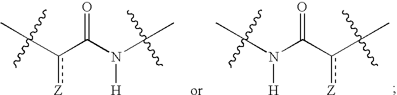

Z is O if [symbol] connected thereto is a double bond or Z is OH if [symbol] connected thereto is a single bond;
X in ring A is selected from the group consisting of N, NH, NR², O, CH, CH₂, CHR², and CR²R²;
each R² is independently selected from the group consisting of alkyl, substituted alkyl, alkenyl, substituted alkenyl, alkynyl, substituted alkynyl, aryl, substituted aryl, heteroaryl, substituted heteroaryl, cycloalkyl, substituted cycloalkyl, heterocyclic, substituted heterocyclic, halo, hydroxy, nitro, cyano, alkoxy, substituted alkoxy, aryloxy, substituted aryloxy, acyl, aminocarbonyl, carboxy, carboxy ester, amino, substituted amino, acylamino, (carboxyl ester)amino, aminocarbonylamino, aminosulfonyl, substituted sulfonyl, and (substituted sulfonyl)amino;
q is 0, 1, 2, 3, or 4;
[symbol] of the ring A indicates a single or double bond;
m is 0, 1, 2, 3, 4, or 5; and
n is 0, 1, 2, 3, 4, or 5;
provided that the compound or pharmaceutically acceptable salt thereof is not
2-hydroxy-2-phenyl-N-(4-(piperidin-1-yl)phenyl)acetamide;
N-(2,4-dimethylphenyl)-2-oxo-2-phenylacetamide;
N1,N2-bis(2-(2-(4-nitrophenylamino)-2-oxoacetyl)phenyl)oxalamide;
2-(2,6-diphenoxyphenyl)-N-(6-methyl-2,4-bis(methylthio)pyridin-3-yl)-2-oxoacetamide;
2-(4-fluorophenyl)-N-(6-methyl-2,4-bis(methylthio)pyridin-3-yl)-2-oxoacetamide;
2-(4-bromophenyl)-N-(6-methyl-2,4-bis(methylthio)pyridin-3-yl)-2-oxoacetamide;
4-(2-(4-(dimethylamino)phenyl)-2-oxoacetamido)benzenesulfonic acid;
2-(2-acetamido-5-bromophenyl)-N-(4-ethoxyphenyl)-2-oxoacetamide;
N-(3-methoxy-4-(oxazol-5-yl)phenyl)-2-oxo-2-phenylacetamide;
2-(2-acetamidophenyl)-N-(4-ethylphenyl)-2-oxoacetamide;
2-(2-acetamidophenyl)-N-(3,5-dichlorophenyl)-2-oxoacetamide;
4-(2-(2-acetamidophenyl)-2-oxoacetamido)-N-phenethylbenzamide;
3-(2-(2-acetamidophenyl)-2-oxoacetamido)-N-(2,4,6-trifluorobenzyl)benzamide;
3-(2-(2-acetamidophenyl)-2-oxoacetamido)-N-(4-chlorophenethyl)benzamide;
2-oxo-N-(4-(4-oxo-4,5,6,7-tetrahydro-1H-pyrrolo[3,2-c]pyridin-2-yl)pyridin-2-yl)-2-phenylacetamide;
2-(2-acetamidophenyl)-N-(3,5-dimethylphenyl)-2-oxoacetamide;
2-(2-acetamidophenyl)-N-(4-(ethylthio)phenyl)-2-oxoacetamide;
2-(4-ethoxydibenzo[b,d]furan-1-yl)-2-oxo-N-(pyridin-4-yl)acetamide;
N-(2-(1-naphthamido)ethyl)-4-(2-(4-nitrophenyl)-2-oxoacetamido)piperidine-4-carboxamide;
2-oxo-N,2-di-p-tolylacetamide; or
3-methyl-2-(2-oxo-2-(p-tolylamino)acetyl)benzenesulfonic acid.

In one embodiment provided is a compound or stereoisomer of Formula II or a pharmaceutically acceptable salt of the compound or the stereoisomer wherein:

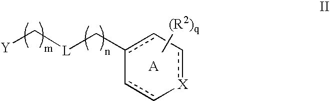

Y is selected from the group consisting of cyclohexyl, substituted cyclohexyl, adamantyl, substituted adamantyl, phenyl, substituted phenyl, heterocyclyl, and substituted heterocyclyl, wherein if Y is substituted cyclohexyl, substituted adamantyl, substituted phenyl, or substituted heterocyclyl, the substituent on Y is selected from the group consisting of alkyl, halo, and haloalkyl;
L is a linker of the formula:

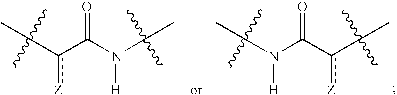

Z is O if [symbol] connected thereto is a double bond or Z is OH if [symbol] connected thereto is a single bond;
X in ring A is selected from the group consisting of N, NH, NR², O, CH, CH₂, CHR², and CR²R²;
each R² is independently selected from the group consisting of alkoxy, substituted alkoxy, aminocarbonyl, heterocyclic, substituted sulfonyl, acyl, carboxy, carboxyl ester, amino, substituted amino, acylamino, (carboxyl ester)amino, aminosulfonyl, (substituted sulfonyl)amino, and haloalkyl;
[symbol] of the ring A indicates a single or double bond;
m is 0, 1, 2, 3, 4, or 5; and
n is 0, 1, 2, 3, 4, or 5.

In some embodiments of Formula (II), Y is cyclohexyl or adamantyl. In some embodiments, Y is selected from the group consisting of 3-trifluoromethylphenyl, 4-trifluoromethoxyphenyl, 3-trifluoromethoxyphenyl, 4-fluoromethylphenyl, 3-fluorophenyl, 4-fluorophenyl.

In some embodiments of Formula (II), Y is substituted cyclohexyl, substituted adamantyl, substituted phenyl, or substituted heterocyclyl, wherein the substituent on Y is selected from the group consisting of alkyl, halo, and haloalkyl.

In some embodiments of Formula (II), each [symbol] of the ring A is a double bond.

In some embodiments of Formula (II), each [symbol] of the ring A is a single bond.

In some embodiments of Formula (II), the ring A is selected from the group consisting of phenyl, piperidinyl, and cyclohexyl.

In some embodiments of Formula (II), each R² is independently selected from the group consisting of alkoxy, substituted alkoxy, aminocarbonyl, haloalkyl, heterocyclic, substituted sulfonyl, acyl, carboxy, carboxyl ester, amino, substituted amino, acylamino, (carboxyl ester)amino, aminosulfonyl, and (substituted sulfonyl)amino.

In some embodiments of Formula (II), q is 1. In some aspects, q is 1, and R² is in the 3-position or 4-position. In some aspects, q is 1, and R² is selected from the group consisting of alkoxy, substituted alkoxy, acyl, carboxy, carboxyl ester, aminocarbonyl, amino, substituted amino, acylamino, (carboxyl ester)amino, aminocarbonylamino, aminosulfonyl, substituted sulfonyl, (substituted sulfonyl)amino, haloalkyl, and heterocyclic.

In some embodiments of Formula (II), Z is O and [symbol] connected thereto is a double bond. In some embodiments of Formula (II), Z is OH and [symbol] connected thereto is a single bond.

In some embodiments of Formula (II), m is 0, 1, or 2. In some embodiments of Formula (II), n is 0, 1, or 2.

In another embodiment, provided is a compound or stereoisomer of Formula III or a pharmaceutically acceptable salt of the compound or the stereoisomer:

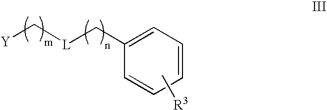

wherein
Y is selected from the group consisting of cyclohexyl, substituted cyclohexyl, adamantyl, substituted adamantyl, phenyl, substituted phenyl, heterocyclyl, substituted heterocyclyl, heteroaryl, and substituted heteroaryl;
L is a linker of the formula:

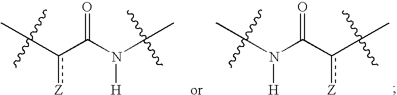

Z is O if [symbol] connected thereto is a double bond or Z is OH if [symbol] connected thereto is a single bond;
R³ is selected from the group consisting of alkyl, substituted alkyl, alkenyl, substituted alkenyl, alkynyl, substituted alkynyl, aryl, substituted aryl, heteroaryl, substituted heteroaryl, cycloalkyl, substituted cycloalkyl, heterocyclic, substituted heterocyclic, halo, hydroxy, nitro, cyano, alkoxy, substituted alkoxy, aryloxy, substituted aryloxy, acyl, aminocarbonyl, carboxy, carboxy ester, amino, substituted amino, acylamino, (carboxyl ester)amino, aminocarbonylamino, aminosulfonyl, substituted sulfonyl, and (substituted sulfonyl)amino;
m is 0, 1, or 2; and
n is 0, 1, or 2;
provided that the compound or pharmaceutically acceptable salt thereof is not
2-hydroxy-2-phenyl-N-(4-(piperidin-1-yl)phenyl)acetamide;
N-(2,4-dimethylphenyl)-2-oxo-2-phenylacetamide;
N1,N2-bis(2-(2-(4-nitrophenylamino)-2-oxoacetyl)phenyl)oxalamide;
2-(2,6-diphenoxyphenyl)-N-(6-methyl-2,4-bis(methylthio)pyridin-3-yl)-2-oxoacetamide;
2-(4-fluorophenyl)-N-(6-methyl-2,4-bis(methylthio)pyridin-3-yl)-2-oxoacetamide;
2-(4-bromophenyl)-N-(6-methyl-2,4-bis(methylthio)pyridin-3-yl)-2-oxoacetamide;
4-(2-(4-(dimethylamino)phenyl)-2-oxoacetamido)benzenesulfonic acid;
2-(2-acetamido-5-bromophenyl)-N-(4-ethoxyphenyl)-2-oxoacetamide;
N-(3-methoxy-4-(oxazol-5-yl)phenyl)-2-oxo-2-phenylacetamide;
2-(2-acetamidophenyl)-N-(4-ethylphenyl)-2-oxoacetamide;
2-(2-acetamidophenyl)-N-(3,5-dichlorophenyl)-2-oxoacetamide;
4-(2-(2-acetamidophenyl)-2-oxoacetamido)-N-phenethylbenzamide;
3-(2-(2-acetamidophenyl)-2-oxoacetamido)-N-(2,4,6-trifluorobenzyl)benzamide;
3-(2-(2-acetamidophenyl)-2-oxoacetamido)-N-(4-chlorophenethyl)benzamide;
2-oxo-N-(4-(4-oxo-4,5,6,7-tetrahydro-1H-pyrrolo[3,2-c]pyridin-2-yl)pyridin-2-yl)-2-phenylacetamide;
2-(2-acetamidophenyl)-N-(3,5-dimethylphenyl)-2-oxoacetamide;
2-(2-acetamidophenyl)-N-(4-(ethylthio)phenyl)-2-oxoacetamide;
2-(4-ethoxydibenzo[b,d]furan-1-yl)-2-oxo-N-(pyridin-4-yl)acetamide;
N-(2-(1-naphthamido)ethyl)-4-(2-(4-nitrophenyl)-2-oxoacetamido)piperidine-4-carboxamide;
2-oxo-N,2-di-p-tolylacetamide; or
3-methyl-2-(2-oxo-2-(p-tolylamino)acetyl)benzenesulfonic acid.

In one embodiment provided is a compound or stereoisomer of Formula III or a pharmaceutically acceptable salt of the compound or the stereoisomer wherein:
Y is selected from the group consisting of cyclohexyl, substituted cyclohexyl, adamantyl, substituted adamantyl, phenyl, substituted phenyl, heterocyclyl, and substituted heterocyclyl, wherein if Y is substituted cyclohexyl, substituted adamantyl, substituted phenyl, or substituted heterocyclyl, the substituent on Y is selected from the group consisting of alkyl, halo, and haloalkyl;
L is a linker of the formula:

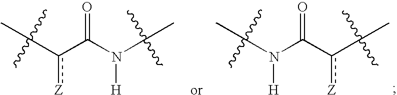

Z is O if [symbol] connected thereto is a double bond or Z is OH if [symbol] connected thereto is a single bond;
each R³ is independently selected from the group consisting of alkoxy, substituted alkoxy, aminocarbonyl, heterocyclic, substituted sulfonyl, acyl, carboxy, carboxyl ester, amino, substituted amino, acylamino, (carboxyl ester)amino, aminosulfonyl, (substituted sulfonyl)amino, and haloalkyl;
m is 0, 1, 2, 3, 4, or 5; and
n is 0, 1, 2, 3, 4, or 5.

In some embodiments, Y is cyclohexyl. In some embodiments, Y is adamantyl. In some embodiments, Y is selected from the group consisting of 3-trifluoromethylphenyl, 4-trifluoromethoxyphenyl, 3-trifluoromethoxyphenyl, 4-fluoromethylphenyl, 3-fluorophenyl, 4-fluorophenyl.

In some embodiments of Formula (III), Y is substituted cyclohexyl, substituted adamantyl, substituted phenyl, or substituted heterocyclyl, wherein the substituent on Y is selected from the group consisting of alkyl, halo, and haloalkyl.

In some embodiments of Formula (III), each R³ is independently selected from the group consisting of alkoxy, substituted alkoxy, aminocarbonyl, haloalkyl, heterocyclic, substituted sulfonyl, acyl, carboxy, carboxyl ester, amino, substituted amino, acylamino, (carboxyl ester)amino, aminosulfonyl, and (substituted sulfonyl)amino.

In some embodiments, R³ is imidazolyl. In some embodiments, R³ is methyl, methoxy, trifluoromethyl, trifluoromethoxy, phenoxy, substituted phenoxy, and benzyloxy.

In some embodiments, R³ is

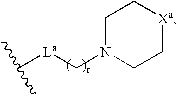

wherein La is —O—, —C(O)—, —C(O)—NH—, r is 0, 1, 2, or 3, and X^{a} is selected from the group consisting of O, S, SO, SO₂, CH₂ and NH.

In some embodiments of Formula (III), Z is O and [symbol] connected thereto is a double bond. In some embodiments of Formula (III), Z is OH and [symbol] connected thereto is a single bond.

In another embodiment, provided is a compound or stereoisomer of Formula IV or a pharmaceutically acceptable salt of the compound or the stereoisomer:

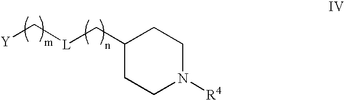

wherein
Y is selected from the group consisting of cyclohexyl, substituted cyclohexyl, adamantyl, substituted adamantyl, phenyl, substituted phenyl, heterocyclyl, substituted heterocyclyl, heteroaryl, and substituted heteroaryl;
L is a linker of the formula:

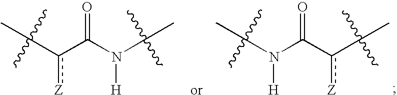

Z is O if [symbol] connected thereto is a double bond or Z is OH if [symbol] connected thereto is a single bond;
R⁴ is selected from the group consisting of alkyl, substituted alkyl, alkenyl, substituted alkenyl, alkynyl, substituted alkynyl, aryl, substituted aryl, heteroaryl, substituted heteroaryl, cycloalkyl, substituted cycloalkyl, heterocyclic, substituted heterocyclic, cyano, acyl, aminocarbonyl, aminosulfonyl, substituted sulfonyl, (substituted sulfonyl)amino, and carboxy ester;
m is 0, 1, or 2; and
n is 0, 1, or 2.

In some embodiments, Y is cyclohexyl. In some embodiments, Y is adamantyl. In some embodiments, Y is selected from the group consisting of 3-trifluoromethylphenyl, 4-trifluoromethoxyphenyl, 3-trifluoromethoxyphenyl, 4-fluoromethylphenyl, 3-fluorophenyl, 4-fluorophenyl.

In some embodiments of Formula (IV), Y is substituted cyclohexyl, substituted adamantyl, substituted phenyl, or substituted heterocyclyl, wherein the substituent on Y is selected from the group consisting of alkyl, halo, and haloalkyl.

In some embodiments of Formula (IV), R⁴ is selected from the group consisting of substituted sulfonyl and acyl. In some embodiments, R¹ is selected from the group consisting of —C(O)-alkyl, —C(O)-substituted alkyl, —C(O)-phenyl, —C(O)-substituted phenyl, —SO₂-alkyl, —SO₂-substituted alkyl, —SO₂-phenyl, —SO₂-substituted phenyl. In some embodiments, the substituted phenyl is selected from the group consisting of tolyl, trifluoromethylphenyl, trifluoromethoxyphenyl, fluorophenyl, and chlorophenyl.

In some embodiments of Formula (IV), Z is O and [symbol] connected thereto is a double bond. In some embodiments of Formula (IV), Z is OH and [symbol] connected thereto is a single bond.

In some aspects of the compounds or compositions of the present invention and subject to the provisos recited herein, provided is a compound, stereoisomer, or a pharmaceutically acceptable salt of the compound or the stereoisomer, wherein the compound is listed in Tables 1 and 2.

TABLE 1

| Compound | Structure | Name |
|---|---|---|
| 1 | 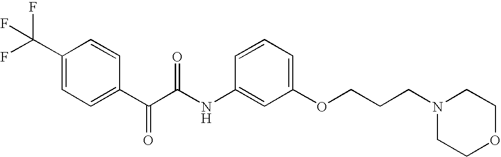 | N-[3-(3-Morpholin-4-yl-propoxy)-phenyl]-2-oxo-2-(4-trifluoromethyl-phenyl)-acetamide |
| 2 | 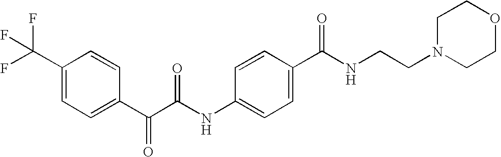 | N-(2-Morpholin-4-yl-ethyl)-4-[2-oxo-2-(4-trifluoromethyl-phenyl)-acetylamino]-benzamide |
| 3 | 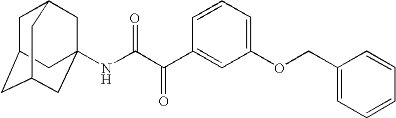 | N-Adamantan-1-yl-2-(3-benzyloxy-phenyl)-2-oxo-acetamide |
| 4 | 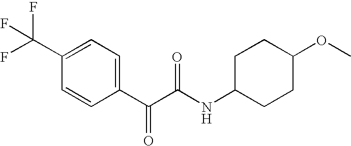 | N-(4-Methoxy-cyclohexyl)-2-oxo-2-(4-trifluoromethyl-phenyl)-acetamide |
| 5 | 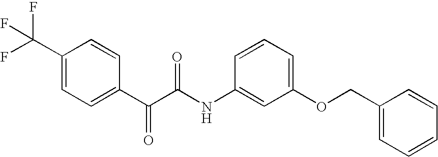 | N-(3-Benzyloxy-phenyl)-2-oxo-2-(4-trifluoromethyl-phenyl)-acetamide |
| 6 | 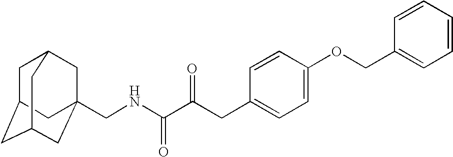 | N-Adamantan-1-ylmethyl-3-(4-benzyloxy-phenyl)-2-oxo-propionamide |
| 7 | 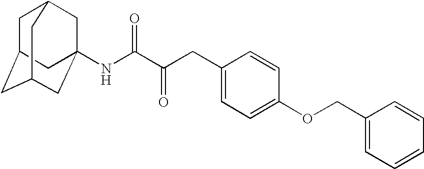 | N-Adamantan-1-yl-3-(4-benzyloxy-phenyl)-2-oxo-propionamide |
| 8 | 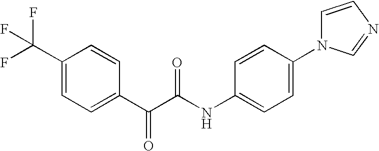 | N-(4-Imidazol-1-yl-phenyl)-2-oxo-2-(4-trifluoromethyl-phenyl)-acetamide |
| 9 | 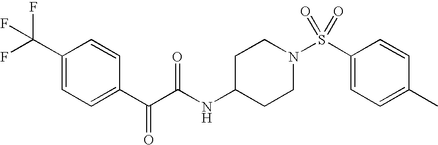 | 2-Oxo-N-[1-(toluene-4-sulfonyl)-piperidin-4-yl]-2-(4-trifluoromethyl-phenyl)-acetamide |
| 10 | 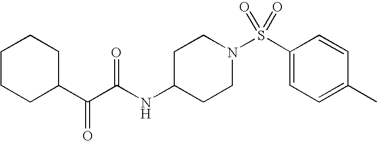 | 2-Cyclohexyl-2-oxo-N-[1-(toluene-4-sulfonyl)-piperidin-4-yl]-acetamide |
| 11 | 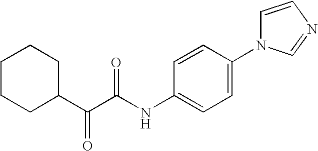 | 2-Cyclohexyl-N-(4-imidazol-1-yl-phenyl)-2-oxo-acetamide |
| 12 | 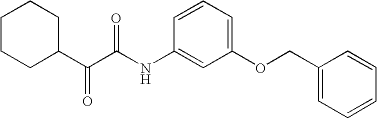 | N-(3-Benzyloxy-phenyl)-2-cyclohexyl-2-oxo-acetamide |
| 13 | 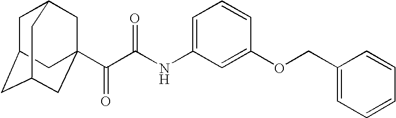 | 2-Adamantan-1-yl-N-(3-benzyloxy-phenyl)-2-oxo-acetamide |
| 14 | 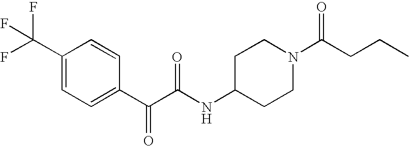 | N-(1-Butyryl-piperidin-4-yl)-2-oxo-2-(4-trifluoromethyl-phenyl)-acetamide |
| 15 | 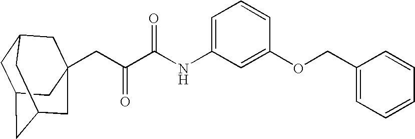 | 3-Adamantan-1-yl-N-(3-benzyloxy-phenyl)-2-oxo-propionamide |
| 16 | 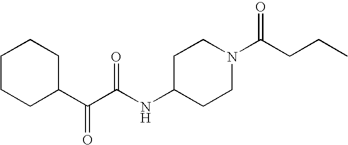 | N-(1-Butyryl-piperidin-4-yl)-2-cyclohexyl-2-oxo-acetamide |
| 17 | 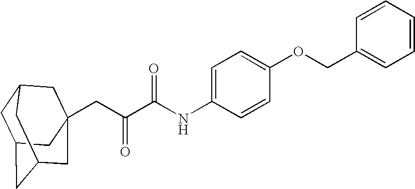 | 3-Adamantan-1-yl-N-(4-benzyloxy-phenyl)-2-oxo-propionamide |
| 18 | 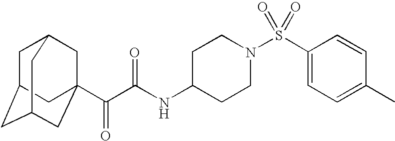 | 2-Adamantan-1-yl-2-oxo-N-[1-(toluene-4-sulfonyl)-piperidin-4-yl]-acetamide |
| 19 | 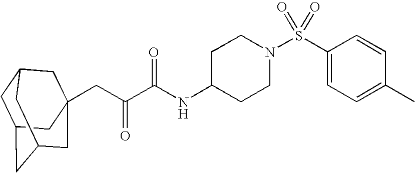 | 3-Adamantan-1-yl-2-oxo-N-[1-(toluene-4-sulfonyl)-piperidin-4-yl]-propionamide |
| 20 | 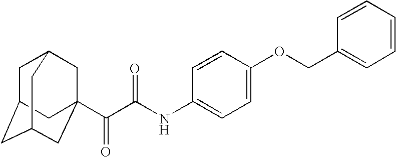 | N-(4-Benzyloxy)phenyl)-2-adamantyl-2-oxoacetamide |
| 21 | 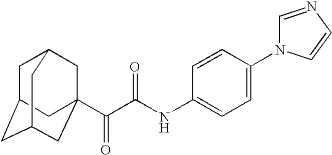 | N-(4-(1H-Imidazol-1-yl)phenyl)-2-adamantyl-2-oxoacetamide |
| 22 | 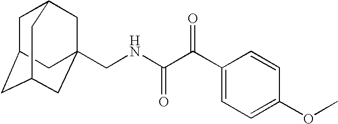 | N-Adamantan-1-ylmethyl)-2-(4-methoxyphenyl)-2-oxoacetamide |
| 23 | 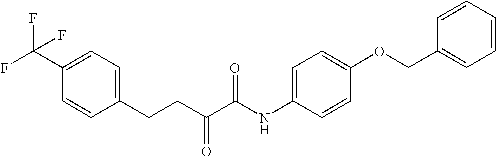 | N-(4-Benzyloxy)phenyl)-2-oxo-4-(4-(trifluoromethyl)phenyl)butanamide |
| 24 | 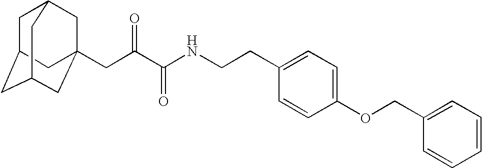 | N-(4-Benzyloxy)phenethyl)-3-adamantan-1-yl-2-oxopropanamide |
| 25 | 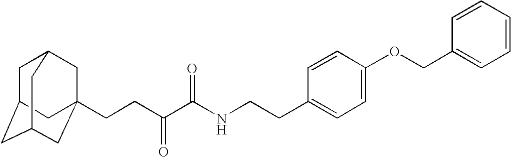 | N-(4-Benzyloxy)phenethyl)-4-adamantyl-2-oxobutanamide |

TABLE 2

| Compound | Structure | Name |
|---|---|---|
| 101 | 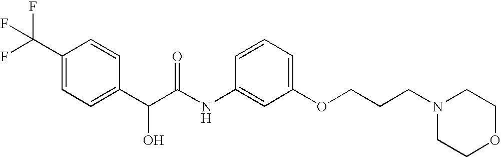 | 2-Hydroxy-N-[3-(3-morpholin-4-yl-propoxy)-phenyl]-2-(4-trifluoromethyl-phenyl)-acetamide |
| 102 | 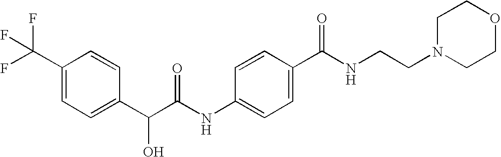 | 4-[2-Hydroxy-2-(4-trifluoromethyl-phenyl)-acetylamino]-N-(2-morpholin-4-yl-ethyl)-benzamide |
| 103 | 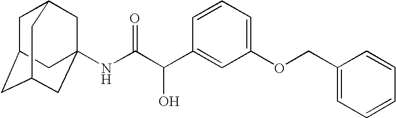 | N-Adamantan-1-yl-2-(3-benzyloxy-phenyl)-2-hydroxy-acetamide |
| 104 | 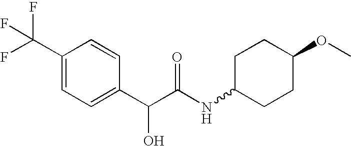 | 2-Hydroxy-N-(4-methoxy-cyclohexyl)-2-(4-trifluoromethyl-phenyl)-acetamide |
| 105 | 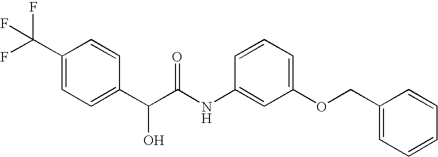 | N-(3-Benzyloxy-phenyl)-2-hydroxy-2-(4-trifluoromethyl-phenyl)-acetamide |
| 106 | 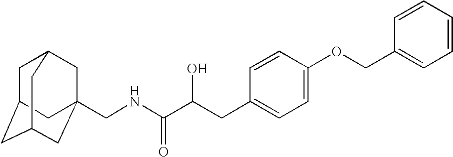 | N-Adamantan-1-ylmethyl-3-(4-benzyloxy-phenyl)-2-hydroxy-propionamide |
| 107 | 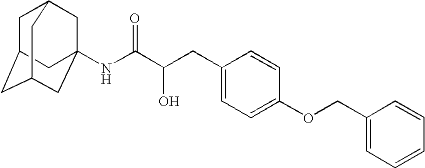 | N-Adamantan-1-yl-3-(4-benzyloxy-phenyl)-2-hydroxy-propionamide |
| 108 | 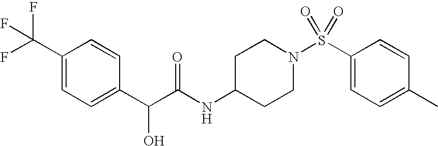 | 2-Hydroxy-N-[1-(toluene-4-sulfonyl)-piperidin-4-yl]-2-(4-trifluoromethyl-phenyl)-acetamide |
| 109 | 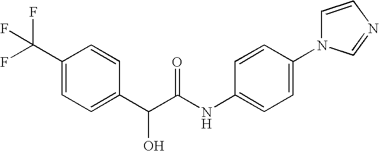 | 2-Hydroxy-N-(4-imidazol-1-yl-phenyl)-2-(4-trifluoromethyl-phenyl)-acetamide |
| 110 | 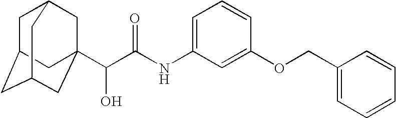 | 2-Adamantan-1-yl-N-(3-benzyloxy-phenyl)-2-hydroxy-acetamide |
| 111 | 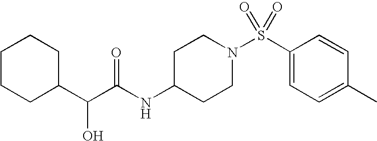 | 2-Cyclohexyl-2-hydroxy-N-[1-(toluene-4-sulfonyl)-piperidin-4-yl]-acetamide |
| 112 | 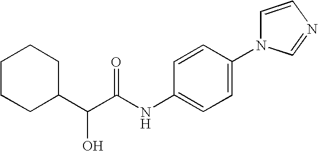 | 2-Cyclohexyl-2-hydroxy-N-(4-imidazol-1-yl-phenyl)-acetamide |
| 113 | 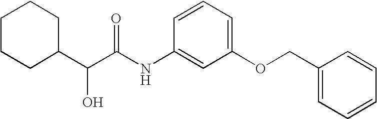 | N-(3-Benzyloxy-phenyl)-2-cyclohexyl-2-hydroxy-acetamide |
| 114 | 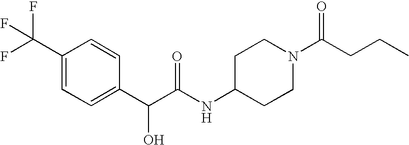 | N-(1-Butyryl-piperidin-4-yl)-2-hydroxy-2-(4-trifluoromethyl-phenyl)-acetamide |
| 115 | 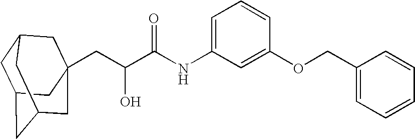 | 3-Adamantan-1-yl-N-(3-benzyloxy-phenyl)-2-hydroxy-propionamide |
| 116 | 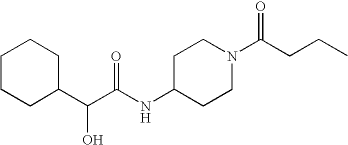 | N-(1-Butyryl-piperidin-4-yl)-2-cyclohexyl-2-hydroxy-acetamide |
| 117 | 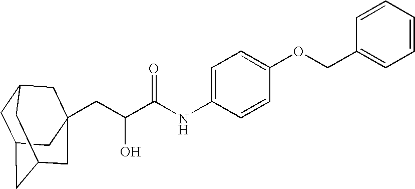 | 3-Adamantan-1-yl-N-(4-benzyloxy-phenyl)-2-hydroxy-propionamide |
| 118 | 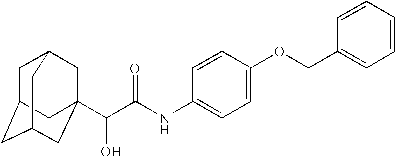 | 2-Adamantan-1-yl-N-(4-benzyloxy-phenyl)-2-hydroxy-acetamide |
| 119 | 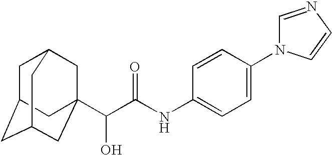 | 2-Adamantan-1-yl-2-hydroxy-N-(4-imidazol-1-yl-phenyl)-acetamide |
| 120 | 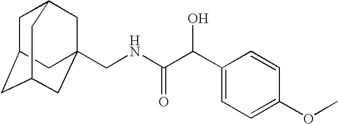 | N-Adamantan-1-ylmethyl-2-hydroxy-2-(4-methoxy-phenyl)-acetamide |
| 121 | 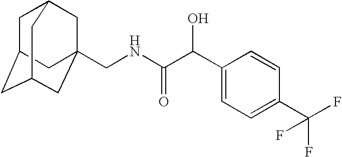 | N-Adamantan-1-ylmethyl-2-hydroxy-2-(4-trifluoromethyl-phenyl)-acetamide |
| 122 | 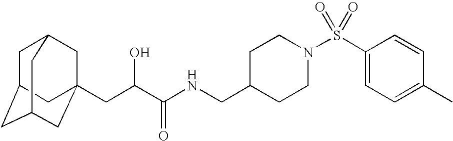 | 3-Adamantan-1-yl-2-hydroxy-N-[1-(toluene-4-sulfonyl)-piperidin-4-ylmethyl]-propionamide |
| 123 | 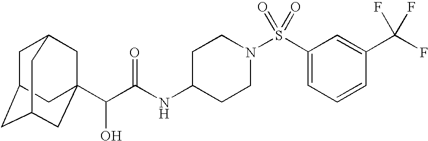 | 2-Adamantan-1-yl-2-hydroxy-N-[1-(3-trifluoromethyl-benzenesulfonyl)-piperidin-4-yl]-acetamide |
| 124 | 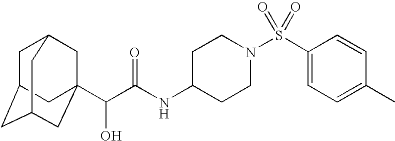 | 2-Adamantan-1-yl-2-hydroxy-N-[1-(toluene-4-sulfonyl)-piperidin-4-yl]-acetamide |
| 125 | 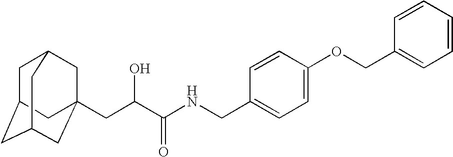 | 3-Adamantan-1-yl-N-(4-benzyloxy-benzyl)-2-hydroxy-propionamide |
| 126 | 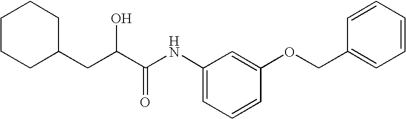 | N-(3-Benzyloxy-phenyl)-3-cyclohexyl-2-hydroxy-propionamide |
| 127 | 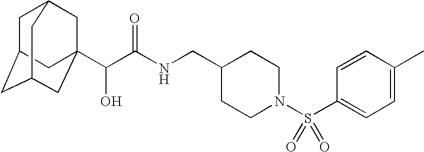 | 2-Adamantan-1-yl-2-hydroxy-N-[1-(toluene-4-sulfonyl)-piperidin-4-ylmethyl]-acetamide |
| 128 | 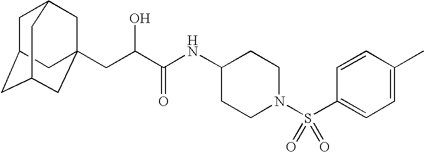 | 3-Adamantan-1-yl-2-hydroxy-N-[1-(toluene-4-sulfonyl)-piperidin-4-yl]-propionamide |
| 129 | 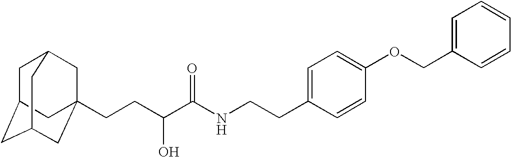 | 4-Adamantan-1-yl-N-[2-(4-benzyloxy-phenyl)-ethyl]-2-hydroxy-butyramide |
| 130 | 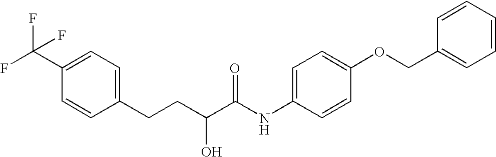 | N-(4-(Benzyloxy)phenyl)-2-hydroxy-4-(4-trifluoromethyl)phenyl)butanamide |
| 131 | 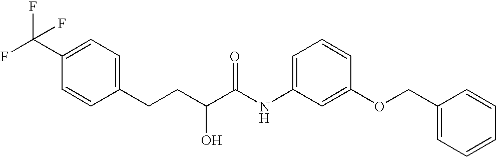 | N-(3-(Benzyloxy)phenyl)-2-hydroxy-4-(4-trifluoromethyl)phenyl)butanamide |
| 132 | 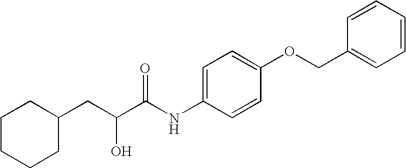 | N-(4-(Benzyloxy)phenyl)-3-cyclohexyl-2-hydroxypropanamide |
| 133 | 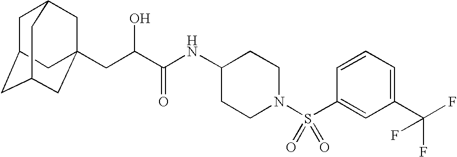 | 3-Adamantan-1-yl-2-hydroxy-N-(1-(3-(trifluoromethyl)phenylsulfonyl)piperidin-4-yl)propanamide |
| 134 | 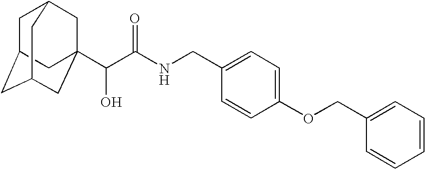 | N-(4-(Benzyloxy)benzyl)-2-adamantan-1-yl-2-hydroxyacetamide |
| 135 | 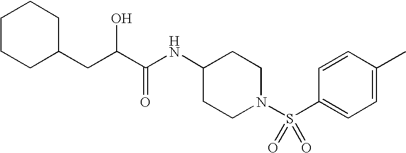 | 3-Cyclohexyl-2-hydroxy-N-(1-tosylpiperidin-4-yl)propanamide |
| 136 | 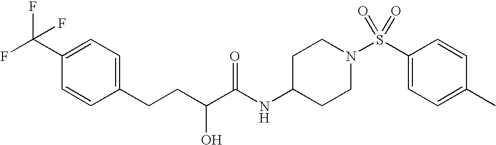 | 2-Hydroxy-N-(1-tosylpiperidin-4-yl)-4-(4-(trifluoromethyl)phenyl)butanamide |
| 137 | 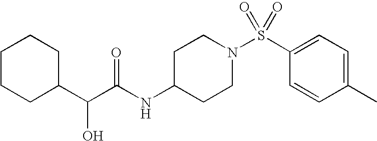 | 2-Cyclohexyl-2-hydroxy-N-(1-tosylpiperidin-4-yl)acetamide |
| 138 | 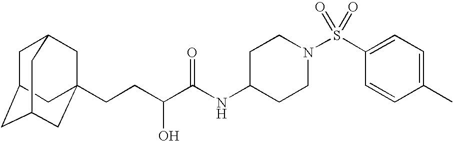 | 4-Adamantan-1-yl-2-hydroxy-N-(1-tosylpiperidin-4-yl)butanamide |
| 139 | 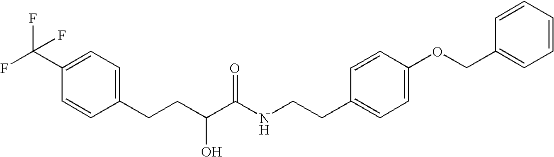 | N-(4-(Benzyloxy)phenethyl)-2-hydroxy-4-(4-(trifluoromethyl)phenyl)butanamide |
| 140 | 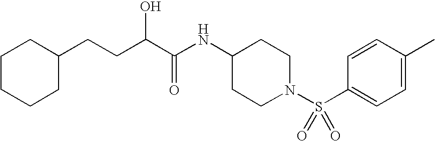 | 4-Cyclohexyl-2-hydroxy-N-(1-tosylpiperidin-4-yl)butanamide |
| 141 | 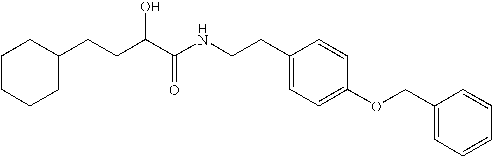 | N-(4-(Benzyloxy)phenethyl)-4-cyclohexyl-2-hydroxybutanamide |
| 142 | 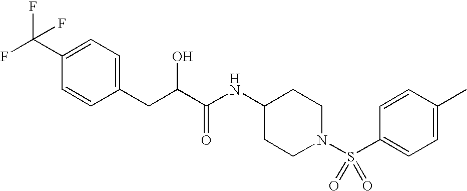 | 2-Hydroxy-N-(1-tosylpiperidin-4-yl)-3-(4-(trifluoromethyl)phenyl)propanamide |
| 143 | 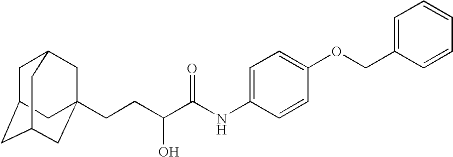 | N-(4-(Benzyloxy)phenyl)-4-adamantan-1-yl-2-hydroxybutanamide |
| 144 | 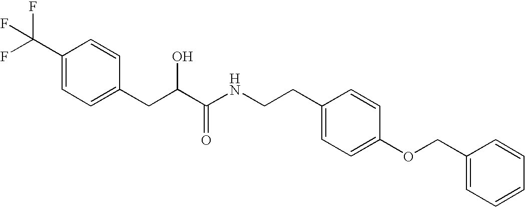 | N-(4-(Benzyloxy)phenethyl)-2-hydroxy-3-(4-(trifluoromethyl)phenyl)propanamide |
| 145 | 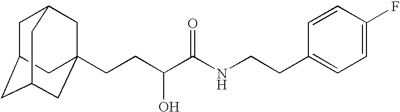 | 4-Adamantan-1-yl-N-(4-fluorophenethyl)-2-hydroxybutanamide |
| 146 | 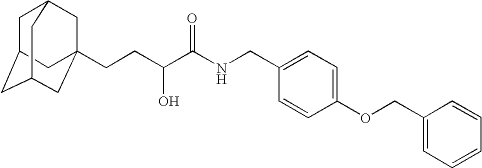 | N-(4-(Benzyloxy)benzyl)-4-adamantyl-2-hydroxybutanamide |
| 147 | 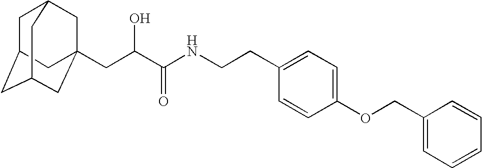 | N-(4-(Benzyloxy)phenethyl)-3-adamantan-1-yl-2-hydroxypropanamide |
| 148 | 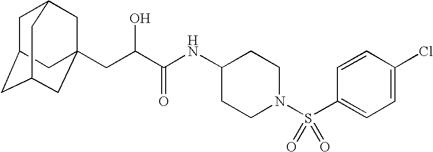 | N-(1-(4-Chlorophenylsulfonyl)piperidin-4-yl)-3-adamantan-1-yl-2-hydroxypropanamide |
| 149 | 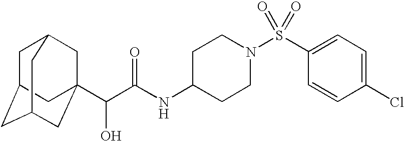 | N-(1-(4-Chlorophenylsulfonyl)piperidin-4-yl)-2-adamantan-1-yl-2-hydroxyacetamide |
| 150 | 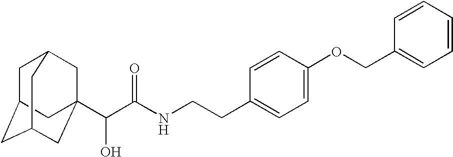 | N-(4-(Benzyloxy)phenethyl)-2-adamantyl-2-hydroxyacetamide |
| 151 | 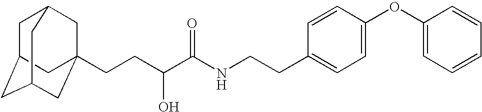 | 4-Adamantan-1-yl-2-hydroxy-N-(4-phenoxyphenethyl)butanamide |
| 152 | 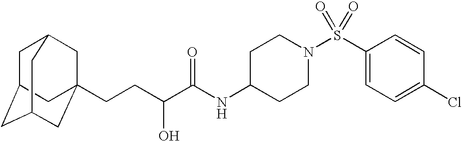 | N-(1-(4-Chlorophenylsulfonyl)piperidin-4-yl)-4-adamantyl-2-hydroxybutanamide |
| 153 | 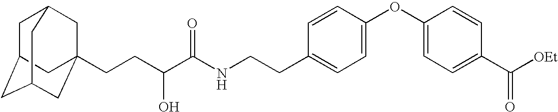 | Ethyl 4-(4-(2-(4-adamantyl-2-hydroxybutanamido)ethyl)phenoxy)benzoate |
| 154 | 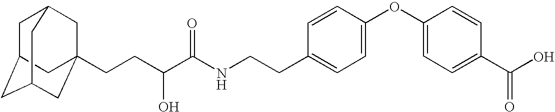 | 4-(4-(2-(4-Adamantan-1-yl-2-hydroxybutanamido)ethyl)phenoxy)benzoic acid |
| 155 | 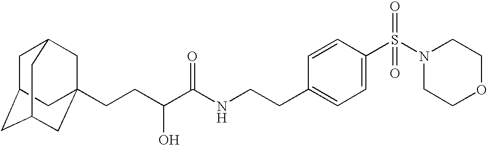 | 4-Adamantan-1-yl-2-hydroxy-N-(4-(morpholinosulfonyl)phenethyl)butanamide |
| 156 | 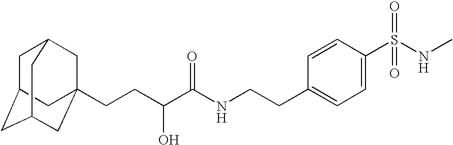 | 4-Adamantan-1-yl-2-hydroxy-N-(4-(N-methylsulfamoyl)phenethyl)butanamide |
| 157 | 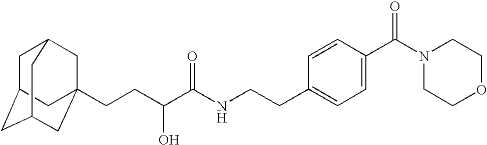 | 4-Adamantan-1-yl-2-hydroxy-N-(4-(morpholine-4-carbonyl)phenethyl)butanamide |

In accordance with another embodiment of the invention, provided is a compound or stereoisomer of Formula V or a pharmaceutically acceptable salt of the compound or the stereoisomer:

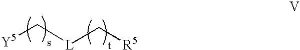

wherein
Y⁵ is selected from the group consisting of cycloalkyl, substituted cycloalkyl, phenyl, substituted phenyl, heterocyclyl, substituted heterocyclyl, heteroaryl, and substituted heteroaryl;
L is a linker of the formula:

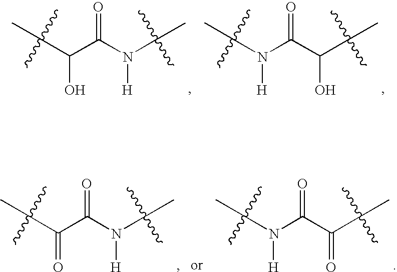

R⁵ is selected from the group consisting of alkyl, alkenyl, alkynyl, hydroxy, nitro, cyano, alkoxy, substituted alkoxy, aryloxy, substituted aryloxy, acyl, aminocarbonyl, carboxy, carboxy ester, amino, substituted amino, acylamino, (carboxyl ester)amino, aminocarbonylamino, aminosulfonyl, substituted sulfonyl, (substituted sulfonyl)amino, phenyl, substituted phenyl, heteroaryl, and substituted heteroaryl;
s is 0, 1, 2, 3, 4, or 5; and
t is 0, 1, 2, 3, 4, 5, 6, 7, 8, 9, 10, 11, 12, or 13.

In some embodiments Y⁵ is cycloalkyl. In some embodiments Y⁵ is adamantyl. In some embodiments Y⁵ is cyclohexyl.

In other embodiments, Y⁵ is phenyl or substituted phenyl. In some embodiments, the phenyl is substituted with halo, haloalkyl or haloalkoxy. In some embodiments Y⁵ is 3-trifluorophenyl or 4-trifluorophenyl. In some embodiments Y⁵ is 3-trifluoromethoxyphenyl or 4-trifluoromethoxyphenyl.

In some embodiments, s is 0, 1, 2, or 3.

In some embodiments, t is 9, 10, 11, or 12.

In some embodiments, R⁵ is carboxy or carboxy ester. In some embodiments, R⁵ is —COOH or —COOCH₃.

In some embodiments, L is:

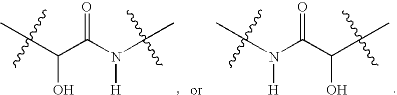

In some embodiments, L is:

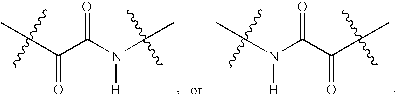

In some embodiments, provided is a compound, stereoisomer, or a pharmaceutically acceptable salt of the compound or the stereoisomer, wherein the compound is listed in Table 3.

TABLE 3

| Compound | Structure | Name |
|---|---|---|
| 158 | 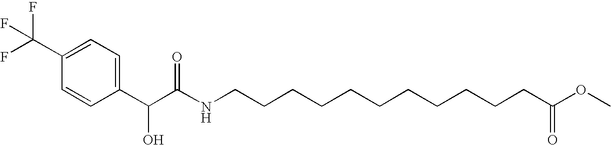 | Methyl 12-(2-hydroxy-2-(4-(trifluoromethyl)phenyl)acetamido)dodecanoate |
| 159 | 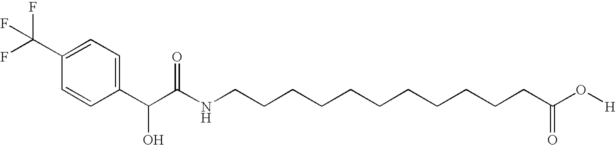 | 12-(2-Hydroxy-2-(4-(trifluoromethyl)phenyl)acetamido)dodecanoic acid |
| 160 | 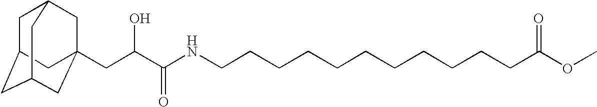 | Methyl 12-(3-adamantan-1-yl-2-hydroxypropanamido)dodecanoate |
| 161 | 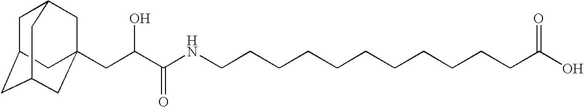 | 12-(3-Adamantan-1-yl-2-hydroxypropanamido)dodecanoic acid |
| 162 | 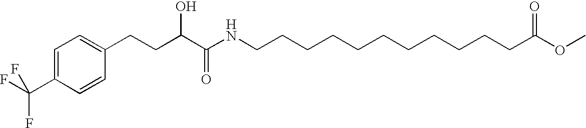 | Methyl 12-(2-hydroxy-4-(4-(trifluoromethyl)phenyl)butanamido)dodecanoate |
| 163 | 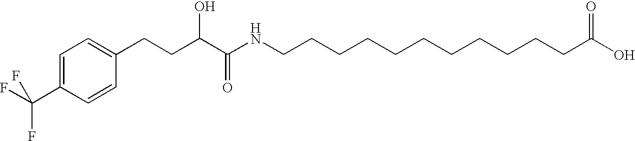 | 12-(2-Hydroxy-4-(4-(trifluoromethyl)phenyl)butanamido)dodecanoicacid |
| 164 | 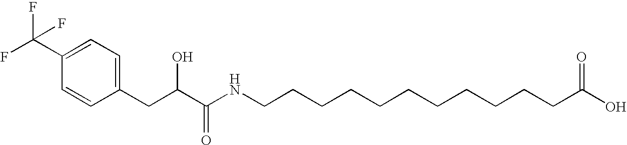 | 12-(2-Hydroxy-3-(4-(trifluoromethyl)phenyl)propanamido)dodecanoicacid |
| 165 | 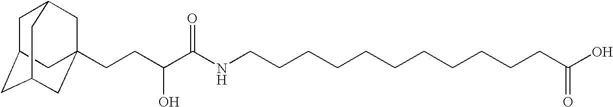 | 12-(4-Adamantan-1-yl-2-hydroxybutanamido)dodecanoic acid |

In one embodiment, provided is a pharmaceutical composition comprising a pharmaceutically acceptable carrier and a therapeutically effective amount of a compound or stereoisomer of any one of Formula A, I, Ia, and II-V or a pharmaceutically acceptable salt of the compound or the stereoisomer for treating a soluble epoxide hydrolase mediated disease.

In another embodiment, provided is a method for treating a soluble epoxide hydrolase mediated disease, said method comprising administering to a patient a pharmaceutical composition comprising a pharmaceutically acceptable carrier and a therapeutically effective amount of a compound or stereoisomer of Formula I or a pharmaceutically acceptable salt of the compound or the stereoisomer:

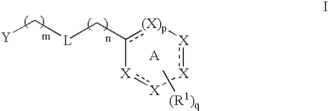

wherein
Y is selected from the group consisting of cycloalkyl, substituted cycloalkyl, phenyl, substituted phenyl, heterocyclyl, substituted heterocyclyl, heteroaryl, and substituted heteroaryl;
L is a linker of the formula:

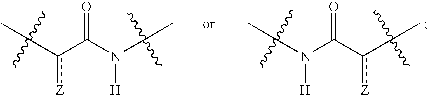

Z is O if [symbol] connected thereto is a double bond or Z is OH if [symbol] connected thereto is a single bond;
each X in ring A is independently selected from the group consisting of N, NH, NR¹, O, CH, CH₂, CHR¹, and CR¹R¹, with the proviso that at least two X's of the A ring are independently CH, CH₂, CHR¹, or CR¹R¹;
p is zero or one;
each R¹ is independently selected from the group consisting of alkyl, substituted alkyl, alkenyl, substituted alkenyl, alkynyl, substituted alkynyl, aryl, substituted aryl, heteroaryl, substituted heteroaryl, cycloalkyl, substituted cycloalkyl, heterocyclic, substituted heterocyclic, halo, hydroxy, nitro, cyano, alkoxy, substituted alkoxy, aryloxy, substituted aryloxy, acyl, aminocarbonyl, carboxy, carboxy ester, amino, substituted amino, acylamino, (carboxyl ester)amino, aminocarbonylamino, aminosulfonyl, substituted sulfonyl, and (substituted sulfonyl)amino;
q is 0, 1, 2, 3, or 4;
[symbol] of the ring A indicates a single or double bond;
m is 0, 1, 2, 3, 4, or 5; and
n is 0, 1, 2, 3, 4, or 5.

In other embodiments, provided is a method for treating a soluble epoxide hydrolase mediated disease, said method comprising administering to a patient a pharmaceutical composition comprising a pharmaceutically acceptable carrier and a therapeutically effective amount of a compound of any one of Formula A, Ia, II-IV or a stereoisomer or pharmaceutically acceptable salt of the compound or the stereoisomer.

In some aspects of the methods, the compound or stereoisomer or pharmaceutically acceptable salt of the compound or the stereoisomer is any one of Compounds 1-25 in Table 1. In some aspects of the methods, the compound or stereoisomer or pharmaceutically acceptable salt of the compound or the stereoisomer is any one of Compounds 101-157 in Table 2.

In another embodiment, provided is a method for treating a soluble epoxide hydrolase mediated disease, said method comprising administering to a patient a pharmaceutical composition comprising a pharmaceutically acceptable carrier and a therapeutically effective amount of a compound or stereoisomer of Formula V or a pharmaceutically acceptable salt of the compound or the stereoisomer:

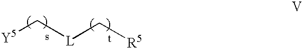

wherein
Y⁵ is selected from the group consisting of cycloalkyl, substituted cycloalkyl, phenyl, substituted phenyl, heterocyclyl, substituted heterocyclyl, heteroaryl, and substituted heteroaryl;
L is a linker of the formula:

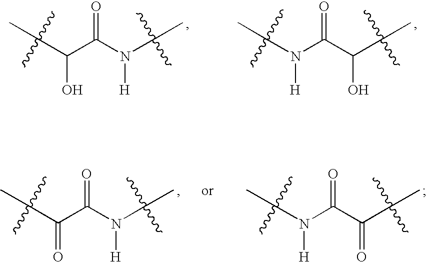

R⁵ is selected from the group consisting of alkyl, alkenyl, alkynyl, hydroxy, nitro, cyano, alkoxy, substituted alkoxy, aryloxy, substituted aryloxy, acyl, aminocarbonyl, carboxy, carboxy ester, amino, substituted amino, acylamino, (carboxyl ester)amino, aminocarbonylamino, aminosulfonyl, substituted sulfonyl, (substituted sulfonyl)amino, phenyl, substituted phenyl, heteroaryl, and substituted heteroaryl;
s is 0, 1, 2, 3, 4, or 5; and
t is 0, 1, 2, 3, 4, 5, 6, 7, 8, 9, 10, 11, 12, or 13.

In some aspects of the methods, the compound or stereoisomer or pharmaceutically acceptable salt of the compound or the stereoisomer is any one of Compounds 158-165 in Table 3.

It has previously been shown that inhibitors of soluble epoxide hydrolase (“sEH”) can reduce hypertension (see, e.g., U.S. Pat. No. 6,351,506). Such inhibitors can be useful in controlling the blood pressure of persons with undesirably high blood pressure, including those who suffer from diabetes.

In preferred embodiments, compounds of the invention are administered to a subject in need of treatment for hypertension, specifically renal, hepatic, or pulmonary hypertension; inflammation, specifically renal inflammation, hepatic inflammation, vascular inflammation, and lung inflammation; adult respiratory distress syndrome; diabetic complications; end stage renal disease; Raynaud syndrome; and arthritis.

### Methods to Treat ARDS and SIRS

Adult respiratory distress syndrome (ARDS) is a pulmonary disease that has a mortality rate of 50% and results from lung lesions that are caused by a variety of conditions found in trauma patients and in severe burn victims. Ingram, R. H. Jr., “Adult Respiratory Distress Syndrome,” Harrison's Principals of Internal Medicine, 13, p. 1240, 1995. With the possible exception of glucocorticoids, there have not been therapeutic agents known to be effective in preventing or ameliorating the tissue injury, such as microvascular damage, associated with acute inflammation that occurs during the early development of ARDS.

ARDS, which is defined in part by the development of alveolar edema, represents a clinical manifestation of pulmonary disease resulting from both direct and indirect lung injury. While previous studies have detailed a seemingly unrelated variety of causative agents, the initial events underlying the pathophysiology of ARDS are not well understood. ARDS was originally viewed as a single organ failure, but is now considered a component of the multisystem organ failure syndrome (MOFS). Pharmacologic intervention or prevention of the inflammatory response is presently viewed as a more promising method of controlling the disease process than improved ventilatory support techniques. See, for example, Demling, Annu. Rev. Med., 46, pp. 193-203, 1995.

Another disease (or group of diseases) involving acute inflammation is the systematic inflammatory response syndrome, or SIRS, which is the designation recently established by a group of researchers to describe related conditions resulting from, for example, sepsis, pancreatitis, multiple trauma such as injury to the brain, and tissue injury, such as laceration of the musculature, brain surgery, hemorrhagic shock, and immune-mediated organ injuries (JAMA, 268(24):3452-3455 (1992)).

The ARDS ailments are seen in a variety of patients with severe burns or sepsis. Sepsis in turn is one of the SIRS symptoms. In ARDS, there is an acute inflammatory reaction with high numbers of neutrophils that migrate into the interstitium and alveoli. If this progresses there is increased inflammation, edema, cell proliferation, and the end result is impaired ability to extract oxygen. ARDS is thus a common complication in a wide variety of diseases and trauma. The only treatment is supportive. There are an estimated 150,000 cases per year and mortality ranges from 10% to 90%.

The exact cause of ARDS is not known. However it has been hypothesized that over-activation of neutrophils leads to the release of linoleic acid in high levels via phospholipase A₂ activity. Linoleic acid in turn is converted to 9,10-epoxy-12-octadecenoate enzymatically by neutrophil cytochrome P-450 epoxygenase and/or a burst of active oxygen. This lipid epoxide, or leukotoxin, is found in high levels in burned skin and in the serum and bronchial lavage of burn patients. Furthermore, when injected into rats, mice, dogs, and other mammals it causes ARDS. The mechanism of action is not known. However, the leukotoxin diol produced by the action of the soluble epoxide hydrolase appears to be a specific inducer of the mitochondrial inner membrane permeability transition (MPT). This induction by leukotoxin diol, the diagnostic release of cytochrome c, nuclear condensation, DNA laddering, and CPP32 activation leading to cell death were all inhibited by cyclosporin A, which is diagnostic for MPT induced cell death. Actions at the mitochondrial and cell level were consistent with this mechanism of action suggesting that the inhibitors of this invention could be used therapeutically with compounds which block MPT.

Thus in one embodiment provided is a method for treating ARDS. In another embodiment, provided is a method for treating SIRS.

### Methods for Inhibiting Progression of Kidney Deterioration (Nephropathy) and Reducing Blood Pressure:

In another aspect of the invention, the compounds of the invention can reduce damage to the kidney, and especially damage to kidneys from diabetes, as measured by albuminuria. The compounds of the invention can reduce kidney deterioration (nephropathy) from diabetes even in individuals who do not have high blood pressure. The conditions of therapeutic administration are as described above.

cis-Epoxyeicosantrienoic acids (“EETs”) can be used in conjunction with the compounds of the invention to further reduce kidney damage. EETs, which are epoxides of arachidonic acid, are known to be effectors of blood pressure, regulators of inflammation, and modulators of vascular permeability. Hydrolysis of the epoxides by sEH diminishes this activity. Inhibition of sEH raises the level of EETs since the rate at which the EETs are hydrolyzed into DHETs is reduced. Without wishing to be bound by theory, it is believed that raising the level of EETs interferes with damage to kidney cells by the microvasculature changes and other pathologic effects of diabetic hyperglycemia. Therefore, raising the EET level in the kidney is believed to protect the kidney from progression from microalbuminuria to end stage renal disease.

EETs are well known in the art. EETs useful in the methods of the present invention include 14,15-EET, 8,9-EET and 11,12-EET, and 5,6 EETs, in that order of preference. Preferably, the EETs are administered as the methyl ester, which is more stable. Persons of skill will recognize that the EETs are regioisomers, such as 8S,9R- and 14R,15S-EET. 8,9-EET, 11,12-EET, and 14R,15S-EET, are commercially available from, for example, Sigma-Aldrich (catalog nos. E5516, E5641, and E5766, respectively, Sigma-Aldrich Corp., St. Louis, Mo.).

EETs produced by the endothelium have anti-hypertensive properties and the EETs 11,12-EET and 14,15-EET may be endothelium-derived hyperpolarizing factors (EDHFs). Additionally, EETs such as 11,12-EET have profibrinolytic effects, anti-inflammatory actions and inhibit smooth muscle cell proliferation and migration. In the context of the present invention, these favorable properties are believed to protect the vasculature and organs during renal and cardiovascular disease states.

Inhibition of sEH activity can be effected by increasing the levels of EETs. This permits EETs to be used in conjunction with one or more sEH inhibitors to reduce nephropathy in the methods of the invention. It further permits EETs to be used in conjunction with one or more sEH inhibitors to reduce hypertension, or inflammation, or both. Thus, medicaments of EETs can be made which can be administered in conjunction with one or more sEH inhibitors, or a medicament containing one or more sEH inhibitors can optionally contain one or more EETs.

The EETs can be administered concurrently with the sEH inhibitor, or following administration of the sEH inhibitor. It is understood that, like all drugs, inhibitors have half lives defined by the rate at which they are metabolized by or excreted from the body, and that the inhibitor will have a period following administration during which it will be present in amounts sufficient to be effective. If EETs are administered after the inhibitor is administered, therefore, it is desirable that the EETs be administered during the period in which the inhibitor will be present in amounts to be effective to delay hydrolysis of the EETs. Typically, the EET or EETs will be administered within 48 hours of administering an sEH inhibitor. Preferably, the EET or EETs are administered within 24 hours of the inhibitor, and even more preferably within 12 hours. In increasing order of desirability, the EET or EETs are administered within 10, 8, 6, 4, 2, hours, 1 hour, or one half hour after administration of the inhibitor. Most preferably, the EET or EETs are administered concurrently with the inhibitor.

In preferred embodiments, the EETs, the compound of the invention, or both, are provided in a material that permits them to be released over time to provide a longer duration of action. Slow release coatings are well known in the pharmaceutical art; the choice of the particular slow release coating is not critical to the practice of the present invention.

EETs are subject to degradation under acidic conditions. Thus, if the EETs are to be administered orally, it is desirable that they are protected from degradation in the stomach. Conveniently, EETs for oral administration may be coated to permit them to passage through the acidic environment of the stomach into the basic environment of the intestines. Such coatings are well known in the art. For example, aspirin coated with so-called “enteric coatings” is widely available commercially. Such enteric coatings may be used to protect EETs during passage through the stomach. An exemplary coating is set forth in the Examples.

While the anti-hypertensive effects of EETs have been recognized, EETs have not been administered to treat hypertension because it was thought endogenous sEH would hydrolyse the EETs too quickly for them to have any useful effect. Surprisingly, it was found during the course of the studies underlying the present invention that exogenously administered inhibitors of sEH succeeded in inhibiting sEH sufficiently that levels of EETs could be further raised by the administration of exogenous EETs. These findings underlie the co-administration of sEH inhibitors and of EETs described above with respect to inhibiting the development and progression of nephropathy. This is an important improvement in augmenting treatment. While levels of endogenous EETs are expected to rise with the inhibition of sEH activity caused by the action of the sEH inhibitor, and therefore to result in at least some improvement in symptoms or pathology, it may not be sufficient in all cases to inhibit progression of kidney damage fully or to the extent intended. This is particularly true where the diseases or other factors have reduced the endogenous concentrations of EETs below those normally present in healthy individuals. Administration of exogenous EETs in conjunction with an sEH inhibitor is therefore expected to be beneficial and to augment the effects of the sEH inhibitor in reducing the progression of diabetic nephropathy.

The present invention can be used with regard to any and all forms of diabetes to the extent that they are associated with progressive damage to the kidney or kidney function. The chronic hyperglycemia of diabetes is associated with long-term damage, dysfunction, and failure of various organs, especially the eyes, kidneys, nerves, heart, and blood vessels. The long-term complications of diabetes include retinopathy with potential loss of vision; nephropathy leading to renal failure; peripheral neuropathy with risk of foot ulcers, amputation, and Charcot joints.

In addition, persons with metabolic syndrome are at high risk of progression to type 2 diabetes, and therefore at higher risk than average for diabetic nephropathy. It is therefore desirable to monitor such individuals for microalbuminuria, and to administer an sEH inhibitor and, optionally, one or more EETs, as an intervention to reduce the development of nephropathy. The practitioner may wait until microalbuminuria is seen before beginning the intervention. Since a person can be diagnosed with metabolic syndrome without having a blood pressure of 130/85 or higher, both persons with blood pressure of 130/85 or higher and persons with blood pressure below 130/85 can benefit from the administration of sEH inhibitors and, optionally, of one or more EETs, to slow the progression of damage to their kidneys. In some preferred embodiments, the person has metabolic syndrome and blood pressure below 130/85.

Dyslipidemia or disorders of lipid metabolism is another risk factor for heart disease. Such disorders include an increased level of LDL cholesterol, a reduced level of HDL cholesterol, and an increased level of triglycerides. An increased level of serum cholesterol, and especially of LDL cholesterol, is associated with an increased risk of heart disease. The kidneys are also damaged by such high levels. It is believed that high levels of triglycerides are associated with kidney damage. In particular, levels of cholesterol over 200 mg/dL, and especially levels over 225 mg/dL, would suggest that sEH inhibitors and, optionally, EETs, should be administered. Similarly, triglyceride levels of more than 215 mg/dL, and especially of 250 mg/dL or higher, would indicate that administration of sEH inhibitors and, optionally, of EETs, would be desirable. The administration of compounds of the present invention with or without the EETs, can reduce the need to administer statin drugs (HMG-COA reductase inhibitors) to the patients, or reduce the amount of the statins needed. In some embodiments, candidates for the methods, uses, and compositions of the invention have triglyceride levels over 215 mg/dL and blood pressure below 130/85. In some embodiments, the candidates have triglyceride levels over 250 mg/dL and blood pressure below 130/85. In some embodiments, candidates for the methods, uses and compositions of the invention have cholesterol levels over 200 mg/dL and blood pressure below 130/85. In some embodiments, the candidates have cholesterol levels over 225 mg/dL and blood pressure below 130/85.

### Methods of Inhibiting the Proliferation of Vascular Smooth Muscle Cells:

In other embodiments, compounds of any one of Formulas A, I, Ia, and II-V inhibit proliferation of vascular smooth muscle (VSM) cells without significant cell toxicity, (e.g. specific to VSM cells). Because VSM cell proliferation is an integral process in the pathophysiology of atherosclerosis, these compounds are suitable for slowing or inhibiting atherosclerosis. These compounds are useful to subjects at risk for atherosclerosis, such as individuals who have diabetes and those who have had a heart attack or a test result showing decreased blood circulation to the heart. The conditions of therapeutic administration are as described above.

The methods of the invention are particularly useful for patients who have had percutaneous intervention, such as angioplasty to reopen a narrowed artery, to reduce or to slow the narrowing of the reopened passage by restenosis. In some preferred embodiments, the artery is a coronary artery. The compounds of the invention can be placed on stents in polymeric coatings to provide a controlled localized release to reduce restenosis. Polymer compositions for implantable medical devices, such as stents, and methods for embedding agents in the polymer for controlled release, are known in the art and taught, for example, in U.S. Pat. Nos. 6,335,029; 6,322,847; 6,299,604; 6,290,722; 6,287,285; and 5,637,113. In preferred embodiments, the coating releases the inhibitor over a period of time, preferably over a period of days, weeks, or months. The particular polymer or other coating chosen is not a critical part of the present invention.

The methods of the invention are useful for slowing or inhibiting the stenosis or restenosis of natural and synthetic vascular grafts. As noted above in connection with stents, desirably, the synthetic vascular graft comprises a material which releases a compound of the invention over time to slow or inhibit VSM proliferation and the consequent stenosis of the graft. Hemodialysis grafts are a particularly preferred embodiment.

In addition to these uses, the methods of the invention can be used to slow or to inhibit stenosis or restenosis of blood vessels of persons who have had a heart attack, or whose test results indicate that they are at risk of a heart attack.

Removal of a clot such as by angioplasty or treatment with tissue plasminogen activator (tPA) can also lead to reperfusion injury, in which the resupply of blood and oxygen to hypoxic cells causes oxidative damage and triggers inflammatory events. In some embodiments, provided are methods for administering the compounds and compositions of the invention for treating reperfusion injury. In some such embodiments, the compounds and compositions are administered prior to or following angioplasty or administration of tPA.

In one group of preferred embodiments, compounds of the invention are administered to reduce proliferation of VSM cells in persons who do not have hypertension. In another group of embodiments, compounds of the invention are used to reduce proliferation of VSM cells in persons who are being treated for hypertension, but with an agent that is not an sEH inhibitor.

The compounds of the invention can be used to interfere with the proliferation of cells which exhibit inappropriate cell cycle regulation. In one important set of embodiments, the cells are cells of a cancer. The proliferation of such cells can be slowed or inhibited by contacting the cells with a compound of the invention. The determination of whether a particular compound of the invention can slow or inhibit the proliferation of cells of any particular type of cancer can be determined using assays routine in the art.

In addition to the use of the compounds of the invention, the levels of EETs can be raised by adding EETs. VSM cells contacted with both an EET and a compound of the invention exhibited slower proliferation than cells exposed to either the EET alone or to the compound of the invention alone. Accordingly, if desired, the slowing or inhibition of VSM cells of a compound of the invention can be enhanced by adding an EET along with a compound of the invention. In the case of stents or vascular grafts, for example, this can conveniently be accomplished by embedding the EET in a coating along with a compound of the invention so that both are released once the stent or graft is in position.

### Methods of Inhibiting the Progression of Obstructive Pulmonary Disease, Interstitial Lung Disease, or Asthma:

Chronic obstructive pulmonary disease, or COPD, encompasses two conditions, emphysema and chronic bronchitis, which relate to damage caused to the lung by air pollution, chronic exposure to chemicals, and tobacco smoke. Emphysema as a disease relates to damage to the alveoli of the lung, which results in loss of the separation between alveoli and a consequent reduction in the overall surface area available for gas exchange. Chronic bronchitis relates to irritation of the bronchioles, resulting in excess production of mucin, and the consequent blocking by mucin of the airways leading to the alveoli. While persons with emphysema do not necessarily have chronic bronchitis or vice versa, it is common for persons with one of the conditions to also have the other, as well as other lung disorders.

Some of the damage to the lungs due to COPD, emphysema, chronic bronchitis, and other obstructive lung disorders can be inhibited or reversed by administering inhibitors of the enzyme known as soluble epoxide hydrolase, or “sEH”. The effects of sEH inhibitors can be increased by also administering EETs. The effect is at least additive over administering the two agents separately, and may indeed be synergistic.

The studies reported herein show that EETs can be used in conjunction with sEH inhibitors to reduce damage to the lungs by tobacco smoke or, by extension, by occupational or environmental irritants. These findings indicate that the co-administration of sEH inhibitors and of EETs can be used to inhibit or slow the development or progression of COPD, emphysema, chronic bronchitis, or other chronic obstructive lung diseases which cause irritation to the lungs.

Animal models of COPD and humans with COPD have elevated levels of immunomodulatory lymphocytes and neutrophils. Neutrophils release agents that cause tissue damage and, if not regulated, will over time have a destructive effect. Without wishing to be bound by theory, it is believed that reducing levels of neutrophils reduces tissue damage contributing to obstructive lung diseases such as COPD, emphysema, and chronic bronchitis. Administration of sEH inhibitors to rats in an animal model of COPD resulted in a reduction in the number of neutrophils found in the lungs. Administration of EETs in addition to the sEH inhibitors also reduced neutrophil levels. The reduction in neutrophil levels in the presence of sEH inhibitor and EETs was greater than in the presence of the sEH inhibitor alone.

While levels of endogenous EETs are expected to rise with the inhibition of sEH activity caused by the action of the sEH inhibitor, and therefore to result in at least some improvement in symptoms or pathology, it may not be sufficient in all cases to inhibit progression of COPD or other pulmonary diseases. This is particularly true where the diseases or other factors have reduced the endogenous concentrations of EETs below those normally present in healthy individuals. Administration of exogenous EETs in conjunction with an sEH inhibitor is therefore expected to augment the effects of the sEH inhibitor in inhibiting or reducing the progression of COPD or other pulmonary diseases.

In addition to inhibiting or reducing the progression of chronic obstructive airway conditions, the invention also provides new ways of reducing the severity or progression of chronic restrictive airway diseases. While obstructive airway diseases tend to result from the destruction of the lung parenchyma, and especially of the alveoli, restrictive diseases tend to arise from the deposition of excess collagen in the parenchyma. These restrictive diseases are commonly referred to as “interstitial lung diseases”, or “ILDs”, and include conditions such as idiopathic pulmonary fibrosis. The methods, compositions, and uses of the invention are useful for reducing the severity or progression of ILDs, such as idiopathic pulmonary fibrosis. Macrophages play a significant role in stimulating interstitial cells, particularly fibroblasts, to lay down collagen. Without wishing to be bound by theory, it is believed that neutrophils are involved in activating macrophages, and that the reduction of neutrophil levels found in the studies reported herein demonstrate that the methods and uses of the invention will also be applicable to reducing the severity and progression of ILDs.

In some preferred embodiments, the ILD is idiopathic pulmonary fibrosis. In other preferred embodiments, the ILD is one associated with an occupational or environmental exposure. Exemplars of such ILDs, are asbestosis, silicosis, coal worker's pneumoconiosis, and berylliosis. Further, occupational exposure to any of a number of inorganic dusts and organic dusts is believed to be associated with mucus hypersecretion and respiratory disease, including cement dust, coke oven emissions, mica, rock dusts, cotton dust, and grain dust (for a more complete list of occupational dusts associated with these conditions, see Table 254-1 of Speizer, “Environmental Lung Diseases,” Harrison's Principles of Internal Medicine, infra, at pp. 1429-1436). In other embodiments, the ILD is sarcoidosis of the lungs. ILDs can also result from radiation in medical treatment, particularly for breast cancer, and from connective tissue or collagen diseases such as rheumatoid arthritis and systemic sclerosis. It is believed that the methods, uses and compositions of the invention can be useful in each of these interstitial lung diseases.

In another set of embodiments, the invention is used to reduce the severity or progression of asthma. Asthma typically results in mucin hypersecretion, resulting in partial airway obstruction. Additionally, irritation of the airway results in the release of mediators which result in airway obstruction. While the lymphocytes and other immunomodulatory cells recruited to the lungs in asthma may differ from those recruited as a result of COPD or an ILD, it is expected that the invention will reduce the influx of immunomodulatory cells, such as neutrophils and eosinophils, and ameliorate the extent of obstruction. Thus, it is expected that the administration of sEH inhibitors, and the administration of sEH inhibitors in combination with EETs, will be useful in reducing airway obstruction due to asthma.

In each of these diseases and conditions, it is believed that at least some of the damage to the lungs is due to agents released by neutrophils which infiltrate into the lungs. The presence of neutrophils in the airways is thus indicative of continuing damage from the disease or condition, while a reduction in the number of neutrophils is indicative of reduced damage or disease progression. Thus, a reduction in the number of neutrophils in the airways in the presence of an agent is a marker that the agent is reducing damage due to the disease or condition, and is slowing the further development of the disease or condition. The number of neutrophils present in the lungs can be determined by, for example, bronchoalveolar lavage.

### Prophylactic and Therapeutic Methods to Reduce Stroke Damage:

Inhibitors of soluble epoxide hydrolase (“sEH”) and EETs administered in conjunction with inhibitors of sEH have been shown to reduce brain damage from strokes. Based on these results, we expect that inhibitors of sEH taken prior to an ischemic stroke will reduce the area of brain damage and will likely reduce the consequent degree of impairment. The reduced area of damage should also be associated with a faster recovery from the effects of the stroke.

While the pathophysiologies of different subtypes of stroke differ, they all cause brain damage. Hemorrhagic stroke differs from ischemic stroke in that the damage is largely due to compression of tissue as blood builds up in the confined space within the skull after a blood vessel ruptures, whereas in ischemic stroke, the damage is largely due to loss of oxygen supply to tissues downstream of the blockage of a blood vessel by a clot. Ischemic strokes are divided into thrombotic strokes, in which a clot blocks a blood vessel in the brain, and embolic strokes, in which a clot formed elsewhere in the body is carried through the blood stream and blocks a vessel there. In both hemorrhagic stroke and ischemic stroke, the damage is due to the death of brain cells. Based on the results observed in our studies, we would expect at least some reduction in brain damage in all types of stroke and in all subtypes.

A number of factors are associated with an increased risk of stroke. Given the results of the studies underlying the present invention, sEH inhibitors administered to persons with any one or more of the following conditions or risk factors: high blood pressure, tobacco use, diabetes, carotid artery disease, peripheral artery disease, atrial fibrillation, transient ischemic attacks (TIAs), blood disorders such as high red blood cell counts and sickle cell disease, high blood cholesterol, obesity, alcohol use of more than one drink a day for women or two drinks a day for men, use of cocaine, a family history of stroke, a previous stroke or heart attack, or being elderly, will reduce the area of brain damaged by a stroke. With respect to being elderly, the risk of stroke increases for every 10 years. Thus, as an individual reaches 60, 70, or 80, administration of sEH inhibitors has an increasingly larger potential benefit. As noted in the next section, the administration of EETs in combination with one or more sEH inhibitors can be beneficial in further reducing the brain damage.

In some preferred uses and methods, the sEH inhibitors and, optionally, EETs, are administered to persons who use tobacco, have carotid artery disease, have peripheral artery disease, have atrial fibrillation, have had one or more transient ischemic attacks (TIAs), have a blood disorder such as a high red blood cell count or sickle cell disease, have high blood cholesterol, are obese, use alcohol in excess of one drink a day if a woman or two drinks a day if a man, use cocaine, have a family history of stroke, have had a previous stroke or heart attack and do not have high blood pressure or diabetes, or are 60, 70, or 80 years of age or more and do not have hypertension or diabetes.

Clot dissolving agents, such as tissue plasminogen activator (tPA), have been shown to reduce the extent of damage from ischemic strokes if administered in the hours shortly after a stroke. For example, tPA is approved by the FDA for use in the first three hours after a stroke. Thus, at least some of the brain damage from a stoke is not instantaneous, but rather occurs over a period of time or after a period of time has elapsed after the stroke. It is contemplated that administration of sEH inhibitors, optionally with EETs, can also reduce brain damage if administered within 6 hours after a stroke has occurred, more preferably within 5, 4, 3, or 2 hours after a stroke has occurred, with each successive shorter interval being more preferable. Even more preferably, the inhibitor or inhibitors are administered 2 hours or less or even 1 hour or less after the stroke, to maximize the reduction in brain damage. Persons of skill are well aware of how to make a diagnosis of whether or not a patient has had a stroke. Such determinations are typically made in hospital emergency rooms, following standard differential diagnosis protocols and imaging procedures.

In some preferred uses and methods, the sEH inhibitors and, optionally, EETs, are administered to persons who have had a stroke within the last 6 hours who: use tobacco, have carotid artery disease, have peripheral artery disease, have atrial fibrillation, have had one or more transient ischemic attacks (TIAs), have a blood disorder such as a high red blood cell count or sickle cell disease, have high blood cholesterol, are obese, use alcohol in excess of one drink a day if a woman or two drinks a day if a man, use cocaine, have a family history of stroke, have had a previous stroke or heart attack and do not have high blood pressure or diabetes, or are 60, 70, or 80 years of age or more and do not have hypertension or diabetes.

### Metabolic Syndrome

Inhibitors of soluble epoxide hydrolase (“sEH”) and EETs administered in conjunction with inhibitors of sEH have been shown to treat one or more conditions associated with metabolic syndrome as provided for in U.S. Provisional Application Ser. No. 60/887,124 which is incorporated herein by reference in its entirety.

Metabolic syndrome is characterized by a group of metabolic risk factors present in one person. The metabolic risk factors include central obesity (excessive fat tissue in and around the abdomen), atherogenic dyslipidemia (blood fat disorders—mainly high triglycerides and low HDL cholesterol), insulin resistance or glucose intolerance, prothrombotic state (e.g., high fibrinogen or plasminogen activator inhibitor in the blood), and high blood pressure (130/85 mmHg or higher).

Metabolic syndrome, in general, can be diagnosed based on the presence of three or more of the following clinical manifestations in one subject:
a) Abdominal obesity characterized by a elevated waist circumference equal to or greater than 40 inches (102 cm) in men and equal to or greater than 35 inches (88 cm) in women;
b) Elevated triglycerides equal to or greater than 150 mg/dL;
c) Reduced levels of high-density lipoproteins of less than 40 mg/dL in women and less than 50 mg/dL in men;
d) High blood pressure equal to or greater than 130/85 mm Hg; and
e) Elevated fasting glucose equal to or greater than 100 mg/dL.

It is desirable to provide early intervention to prevent the onset of metabolic syndrome so as to avoid the medical complications brought on by this syndrome. Prevention or inhibition of metabolic syndrome refers to early intervention in subjects predisposed to, but not yet manifesting, metabolic syndrome. These subjects may have a genetic disposition associated with metabolic syndrome and/or they may have certain external acquired factors associated with metabolic syndrome, such as excess body fat, poor diet, and physical inactivity. Additionally, these subjects may exhibit one or more of the conditions associated with metabolic syndrome. These conditions can be in their incipient form.

Accordingly, one aspect, the invention provides a method for inhibiting the onset of metabolic syndrome by administering to the subject predisposed thereto an effective amount of a sEH inhibitor.

Another aspect provides a method for treating one or more conditions associated with metabolic syndrome in a subject where the conditions are selected from incipient diabetes, obesity, glucose intolerance, high blood pressure, elevated serum cholesterol, and elevated triglycerides. This method comprises administering to the subject an amount of an sEH inhibitor effective to treat the condition or conditions manifested in the subject. In one embodiment of this aspect, two or more of the noted conditions are treated by administering to the subject an effective amount of an sEH inhibitor. In this aspect, the conditions to be treated include treatment of hypertension.

sEH inhibitors are also useful in treating metabolic conditions comprising obesity, glucose intolerance, hypertension, high blood pressure, elevated levels of serum cholesterol, and elevated levels of triglycerides, or combinations thereof, regardless if the subject is manifesting, or is predisposed to, metabolic syndrome.

Accordingly, another aspect of the invention provides for methods for treating a metabolic condition in a subject, comprising administering to the subject an effective amount of a sEH inhibitor, wherein the metabolic condition is selected from the group consisting of conditions comprising obesity, glucose intolerance, high blood pressure, elevated serum cholesterol, and elevated triglycerides, and combinations thereof.

In general, levels of glucose, serum cholesterol, triglycerides, obesity, and blood pressure are well known parameters and are readily determined using methods known in the art.

Several distinct categories of glucose intolerance exist, including for example, type 1 diabetes mellitus, type 2 diabetes mellitus, gestational diabetes mellitus (GDM), impaired glucose tolerance (IGT), and impaired fasting glucose (IFG). IGT and IFG are transitional states from a state of normal glycemia to diabetes. IGT is defined as two-hour glucose levels of 140 to 199 mg per dL (7.8 to 11.0 mmol) on the 75-g oral glucose tolerance test (OGTT), and IFG is defined as fasting plasma glucose (FG) values of 100 to 125 mg per dL (5.6 to 6.9 mmol per L) in fasting patients. These glucose levels are above normal but below the level that is diagnostic for diabetes. Rao, et al., Amer. Fam. Phys. 69:1961-1968 (2004).

Current knowledge suggests that development of glucose intolerance or diabetes is initiated by insulin resistance and is worsened by the compensatory hyperinsulinemia. The progression to type 2 diabetes is influenced by genetics and environmental or acquired factors including, for example, a sedentary lifestyle and poor dietary habits that promote obesity. Patients with type 2 diabetes are usually obese, and obesity is also associated with insulin resistance.

“Incipient diabetes” refers to a state where a subject has elevated levels of glucose or, alternatively, elevated levels of glycosylated hemoglobin, but has not developed diabetes. A standard measure of the long term severity and progression of diabetes in a patient is the concentration of glycosylated proteins, typically glycosylated hemoglobin. Glycosylated proteins are formed by the spontaneous reaction of glucose with a free amino group, typically the N-terminal amino group, of a protein. HbA1c is one specific type of glycosylated hemoglobin (Hb), constituting approximately 80% of all glycosylated hemoglobin, in which the N-terminal amino group of the Hb A beta chain is glycosylated.

Formation of HbA1c irreversible and the blood level depends on both the life span of the red blood cells (average 120 days) and the blood glucose concentration. A buildup of glycosylated hemoglobin within the red cell reflects the average level of glucose to which the cell has been exposed during its life cycle. Thus the amount of glycosylated hemoglobin can be indicative of the effectiveness of therapy by monitoring long-term serum glucose regulation. The HbA1c level is proportional to average blood glucose concentration over the previous four weeks to three months. Therefore HbA1c represents the time-averaged blood glucose values, and is not subject to the wide fluctuations observed in blood glucose values, a measurement most typically taken in conjunction with clinical trials of candidate drugs for controlling diabetes.

Obesity can be monitored by measuring the weight of a subject or by measuring the Body Mass Index (BMI) of a subject. BMI is determined by dividing the subject's weight in kilograms by the square of his/her height in metres (BMI=kg/m2). Alternatively, obesity can be monitored by measuring percent body fat. Percent body fat can be measured by methods known in the art including by weighing a subject underwater, by a skinfold test, in which a pinch of skin is precisely measured to determine the thickness of the subcutaneous fat layer, or by bioelectrical impedance analysis.

### Combination Therapy

As noted above, the compounds of the present invention will, in some instances, be used in combination with other therapeutic agents to bring about a desired effect. Selection of additional agents will, in large part, depend on the desired target therapy (see, e.g., Turner, N. et al. Prog. Drug Res. (1998) 51: 33-94; Haffner, S. Diabetes Care (1998) 21: 160-178; and DeFronzo, R. et al. (eds), Diabetes Reviews (1997) Vol. 5 No. 4). A number of studies have investigated the benefits of combination therapies with oral agents (see, e.g., Mahler, R., J. Clin. Endocrinol. Metab. (1999) 84: 1165-71; United Kingdom Prospective Diabetes Study Group: UKPDS 28, Diabetes Care (1998) 21: 87-92; Bardin, C. W., (ed), Current Therapy In Endocrinology And Metabolism, 6th Edition (Mosby-Year Book, Inc., St. Louis, Mo. 1997); Chiasson, J. et al., Ann. Intern. Med. (1994) 121: 928-935; Coniff, R. et al., Clin. Ther. (1997) 19: 16-26; Coniff, R. et al., Am. J. Med. (1995) 98: 443-451; and Iwamoto, Y. et al., Diabet. Med. (1996) 13 365-370; Kwiterovich, P. Am. J. Cardiol (1998) 82(12A): 3U-17U). Combination therapy includes administration of a single pharmaceutical dosage formulation which contains a compound of any one of Formulas A, I, Ia, and II-V and one or more additional active agents, as well as administration of the compound and each active agent in its own separate pharmaceutical dosage formulation. For example, a compound of any one of Formulas A, I, Ia, and II-V and one or more angiotensin receptor blockers, angiotensin converting enzyme inhibitors, calcium channel blockers, diuretics, alpha blockers, beta blockers, centrally acting agents, vasopeptidase inhibitors, renin inhibitors, endothelin receptor agonists, AGE (advanced glycation end-products) crosslink breakers, sodium/potassium ATPase inhibitors, endothelin receptor agonists, endothelin receptor antagonists, angiotensin vaccine, and the like; can be administered to the human subject together in a single oral dosage composition, such as a tablet or capsule, or each agent can be administered in separate oral dosage formulations. Where separate dosage formulations are used, the compound of any one of Formulas A, I, Ia, and II-V and one or more additional active agents can be administered at essentially the same time (i.e., concurrently), or at separately staggered times (i.e., sequentially). Combination therapy is understood to include all these regimens.

### Administration and Pharmaceutical Composition

In general, the compounds of this invention will be administered in a therapeutically effective amount by any of the accepted modes of administration for agents that serve similar utilities. The actual amount of the compound of this invention, i.e., the active ingredient, will depend upon numerous factors such as the severity of the disease to be treated, the age and relative health of the subject, the potency of the compound used, the route and form of administration, and other factors. The drug can be administered more than once a day, preferably once or twice a day. All of these factors are within the skill of the attending clinician.

Therapeutically effective amounts of the compounds may range from approximately 0.05 to 50 mg per kilogram body weight of the recipient per day; preferably about 0.1-25 mg/kg/day, more preferably from about 0.5 to 10 mg/kg/day. Thus, for administration to a 70 kg person, the dosage range would most preferably be about 35-70 mg per day.

In general, compounds of this invention will be administered as pharmaceutical compositions by any one of the following routes: oral, systemic (e.g., transdermal, intranasal or by suppository), parenteral (e.g., intramuscular, intravenous or subcutaneous), or intrathecal administration. The preferred manner of administration is oral using a convenient daily dosage regimen that can be adjusted according to the degree of affliction. Compositions can take the form of tablets, pills, capsules, semisolids, powders, sustained release formulations, solutions, suspensions, elixirs, aerosols, or any other appropriate compositions. Another preferred manner for administering compounds of this invention is inhalation. This is an effective method for delivering a therapeutic agent directly to the respiratory tract (see U.S. Pat. No. 5,607,915).

The choice of formulation depends on various factors such as the mode of drug administration and bioavailability of the drug substance. For delivery via inhalation the compound can be formulated as liquid solution, suspensions, aerosol propellants or dry powder and loaded into a suitable dispenser for administration. There are several types of pharmaceutical inhalation devices-nebulizer inhalers, metered dose inhalers (MDI) and dry powder inhalers (DPI). Nebulizer devices produce a stream of high velocity air that causes the therapeutic agents (which are formulated in a liquid form) to spray as a mist that is carried into the patient's respiratory tract. MDI's typically are formulation packaged with a compressed gas. Upon actuation, the device discharges a measured amount of therapeutic agent by compressed gas, thus affording a reliable method of administering a set amount of agent. DPI dispenses therapeutic agents in the form of a free flowing powder that can be dispersed in the patient's inspiratory air-stream during breathing by the device. In order to achieve a free flowing powder, the therapeutic agent is formulated with an excipient such as lactose. A measured amount of the therapeutic agent is stored in a capsule form and is dispensed with each actuation.

Recently, pharmaceutical formulations have been developed especially for drugs that show poor bioavailability based upon the principle that bioavailability can be increased by increasing the surface area, i.e., decreasing particle size. For example, U.S. Pat. No. 4,107,288 describes a pharmaceutical formulation having particles in the size range from 10 to 1,000 nm in which the active material is supported on a crosslinked matrix of macromolecules. U.S. Pat. No. 5,145,684 describes the production of a pharmaceutical formulation in which the drug substance is pulverized to nanoparticles (average particle size of 400 nm) in the presence of a surface modifier and then dispersed in a liquid medium to give a pharmaceutical formulation that exhibits remarkably high bioavailability.

The compositions are comprised of in general, a compound of the invention in combination with at least one pharmaceutically acceptable excipient. Acceptable excipients are non-toxic, aid administration, and do not adversely affect the therapeutic benefit of the compound. Such excipient may be any solid, liquid, semi-solid or, in the case of an aerosol composition, gaseous excipient that is generally available to one of skill in the art.

Solid pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients may be selected from glycerol, propylene glycol, water, ethanol and various oils, including those of petroleum, animal, vegetable or synthetic origin, e.g., peanut oil, soybean oil, mineral oil, sesame oil, etc. Preferred liquid carriers, particularly for injectable solutions, include water, saline, aqueous dextrose, and glycols.

Compressed gases may be used to disperse a compound of this invention in aerosol form. Inert gases suitable for this purpose are nitrogen, carbon dioxide, etc. Other suitable pharmaceutical excipients and their formulations are described in Remington's Pharmaceutical Sciences, edited by E. W. Martin (Mack Publishing Company, 18th ed., 1990).

The amount of the compound in a formulation can vary within the full range employed by those skilled in the art. Typically, the formulation will contain, on a weight percent (wt %) basis, from about 0.01-99.99 wt % of the compound of based on the total formulation, with the balance being one or more suitable pharmaceutical excipients. Preferably, the compound is present at a level of about 1-80 wt %. Representative pharmaceutical formulations containing a compound of any one of Formulas A, I, Ia, and II-V are described below.

### General Synthetic Methods

The compounds of this invention can be prepared from readily available starting materials using the following general methods and procedures. It will be appreciated that where typical or preferred process conditions (i.e., reaction temperatures, times, mole ratios of reactants, solvents, pressures, etc) are given, other process conditions can also be used unless otherwise stated. Optimum reaction conditions may vary with the particular reactants or solvent used, but such conditions can be determined by one skilled in the art by routine optimization procedures.

Additionally, as will be apparent to those skilled in the art, conventional protecting groups may be necessary to prevent certain functional groups from undergoing undesired reactions. Suitable protecting groups for various functional groups as well as suitable conditions for protecting and deprotecting particular functional groups are well known in the art. For example, numerous protecting groups are described in T. W. Greene and G. M. Wuts, Protecting Groups in Organic Synthesis, Third Edition, Wiley, New York, 1999, and references cited therein.

Furthermore, the compounds of this invention may contain one or more chiral centers. Accordingly, if desired, such compounds can be prepared or isolated as pure stereoisomers, i.e., as individual enantiomers or diastereomers, or as stereoisomer-enriched mixtures. All such stereoisomers (and enriched mixtures) are included within the scope of this invention, unless otherwise indicated. Pure stereoisomers (or enriched mixtures) may be prepared using, for example, optically active starting materials or stereoselective reagents well-known in the art. Alternatively, racemic mixtures of such compounds can be separated using, for example, chiral column chromatography, chiral resolving agents and the like.

The starting materials for the following reactions are generally known compounds or can be prepared by known procedures or obvious modifications thereof. For example, many of the starting materials are available from commercial suppliers such as Aldrich Chemical Co. (Milwaukee, Wis., USA), Bachem (Torrance, Calif., USA), Emka-Chemce or Sigma (St. Louis, Mo., USA). Others may be prepared by procedures, or obvious modifications thereof, described in standard reference texts such as Fieser and Fieser's Reagents for Organic Synthesis, Volumes 1-15 (John Wiley and Sons, 1991), Rodd's Chemistry of Carbon Compounds, Volumes 1-5 and Supplementals (Elsevier Science Publishers, 1989), Organic Reactions, Volumes 1-40 (John Wiley and Sons, 1991), March's Advanced Organic Chemistry, (John Wiley and Sons, 4^{th} Edition), and Larock's Comprehensive Organic Transformations (VCH Publishers Inc., 1989).

The various starting materials, intermediates, and compounds of the invention may be isolated and purified where appropriate using conventional techniques such as precipitation, filtration, crystallization, evaporation, distillation, and chromatography. Characterization of these compounds may be performed using conventional methods such as by melting point, mass spectrum, nuclear magnetic resonance, and various other spectroscopic analyses.

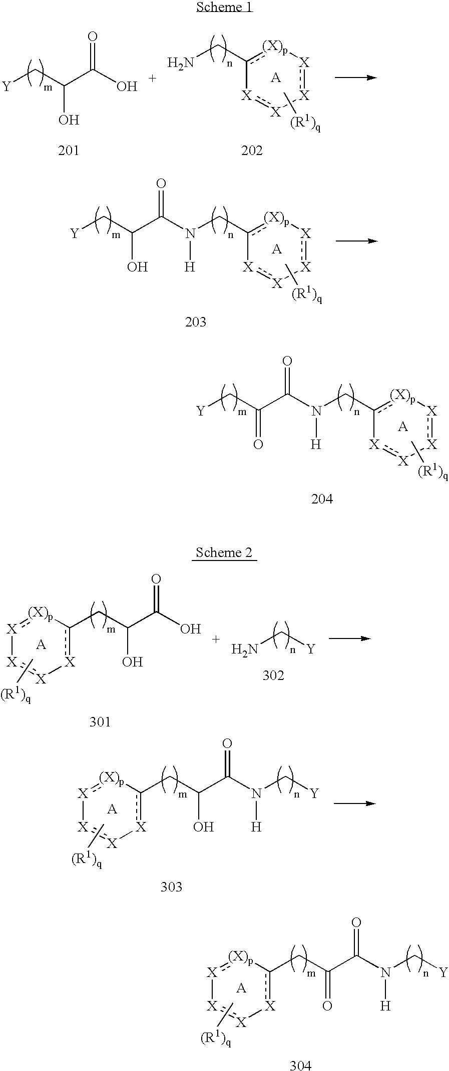

A synthesis of the compounds of the invention is shown in Schemes 1 and 2, where X, Y, R¹, p, q, A, [symbol] m, and n are previously defined. Substituent R¹ can be present initially in starting materials 202 and 301 or converted from other functional groups using conventional synthetic techniques.

In Scheme 1, Compounds 201 and 202 are coupled under standard amide coupling reaction conditions to produce Compound 203. In Scheme 2, Compounds 301 and 302 are coupled under standard amide coupling reaction conditions to produce Compound 303. In certain aspects, a coupling agent is used, such as 1-hydroxybenzotriazole (HOBT) and 1-ethyl-3-(3′-dimethylaminopropyl)carbodiimide (EDCI). Other suitable coupling reagents include benzotriazol-1-yloxy tripyrrolidinophosphonium hexafluorophosphate (PyBOP), 1-hydroxy-7-azabenzotriazole (HOAT), O-(7-azabenzotriazol-1-yl)-N,N,N′,N′-tetramethyluronium hexafluorophosphate (HATU); bis(2-oxo-3-oxazolidinyl)phosphinic chloride (BOP-Cl); 1-(3-dimethylaminopropyl)-3-ethylcarbodiimide hydrochloride (EDAC); and the like. Conversion to acid chlorides to facilitate coupling to form the corresponding amides can also be performed.

Compounds 203 and 303 are representative of alpha hydroxylamide compounds of the preferred embodiments. Compounds 203 and 303 can each be further subjected to Dess Martin oxidation to yield Compounds 204 and 304, respectively, which are representative of alpha keto amid compounds of the preferred embodiments. In Dess Martin oxidation, Compound 203 or 303 is contacted with Dess Martin periodinane or Dess Martin reagent. The oxidation can be performed in dichloromethane or acetone at room temperature.

Compounds of Formula (V) may be prepared using procedure similar to those described above and exemplified below.

The following examples are provided to illustrate certain aspects of the present invention and to aid those of skill in the art in practicing the invention. These examples are in no way to be considered to limit the scope of the invention.

### EXAMPLES

The examples below as well as throughout the application, the following abbreviations have the following meanings. If not defined, the terms have their generally accepted meanings.
aq.=aqueous
brs=broad singlet
d=doublet
DCM=dichloromethane
DIPEA=diisopropylethylamine
DMF=dimethylformamide
DMSO=dimethylsulfoxide
g=gram
HCl=hydrochloric acid
HPLC=high pressure liquid chromatography
LCMS=liquid chromatography mass spectroscopy
m=multiplet
MHz=megahertz
mL=milliliter
m.p.=melting point
N=normal
s=singlet
t=triplet
TEA=triethylamine
THF=tetrahydrofuran
TLC=thin layer chromatography
Example 1

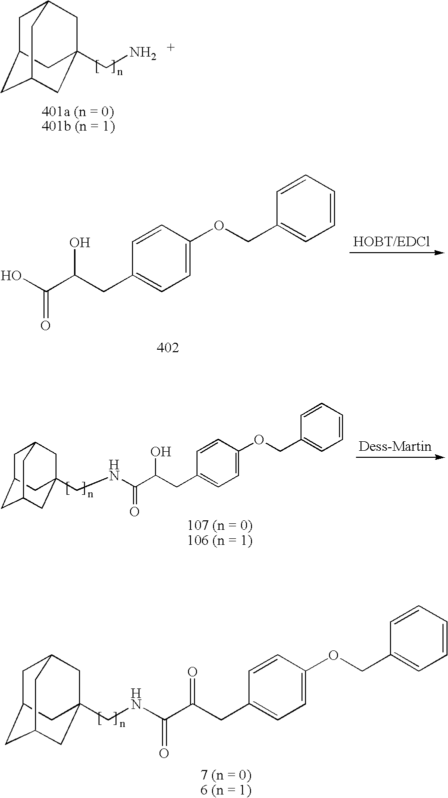

### Synthesis of Compound 106

To a solution of adamantylmethyl amine 401b (0.33 g, 2.0 mmol) in dichloromethane (10 mL) was added hydroxyl acid 402 (0.544 g, 2.00 mmol), hydroxybenzotriazole (HOBT) (0.27 g, 2.0 mmol), N-ethyl-N′-(3-dimethylaminopropyl)carbodiimide (EDCI) (0.383 g, 2.00 mmol) and diisopropylethyl amine (0.515 g, 4.00 mmol), and the resulting mixture was stirred at room temperature overnight. The reaction mixture was then extracted with dichloromethane, washed with saturated sodium bicarbonate solution (50 mL) followed by aqueous hydrochloric acid (10%, 50 mL). The residue obtained after drying the organic portion with sodium sulfate and removal of solvent was chromatographed on silica gel eluting with methanol (5%) in DCM to obtain pure product 106 (400 mg, 48%). ¹H NMR (CDCl₃, 300 MHz): δ 7.42 (m, 5H), 7.20 (d, J=8.1 Hz, 2H), 6.96 (d, J=8.1 Hz, 2H), 6.50 (bs, 1H), 5.05 (s, 2H), 4.32 (m, 1H), 3.20 (dd, J=3.9 Hz, 1H), 2.88-3.01 (m, 3H), 1.98 (s, 3H), 1.60-1.74 (m, 6H), 1.43 (s, 6H). MS: 420 (M+1). HPLC purity=98.1%.

### Synthesis of Compound 107

Synthesis of Compound 107 was carried out in a similar fashion to the above preparation using adamantyl amine (355 mg, 87%). ¹H NMR (300 MHz, CDCl₃): δ 7.47-7.32 (m, 5H), 7.16 (d, 2H, J=4.5 Hz), 6.95 (d, 2H, J=4.5 Hz), 5.92 (s, 1H, NH), 5.07 (s, 2H), 4.19-4.09 (m, 1H), 3.13-2.74 (m, 2H), 2.09-1.65 (m, 15H). MS: 406 [M+1]. HPLC purity 90.2%.

### Synthesis of Compound 6

To a solution of Compound 106 (0.10 g, 0.23 mmol) in DCM (10 mL) was added Dess-Martin periodinane (0.10 g, 0.23 mmol), and the resulting mixture was stirred at room temperature for 2 hours. The reaction mixture was diluted with ether (50 mL), and the resulting suspension was added to a saturated aqueous sodium bicarbonate solution containing sodium thiosulfate and stirred at room temperature for 15 minutes. The ether layer was then separated and washed with saturated aqueous bicarbonate and water. The crude product obtained on removal of solvent was purified by chromatography on silica gel eluting with dichloromethane to obtain the product 6 as a white crystalline solid (40 mg, 42%). ¹H NMR (CDCl₃, 300 MHz): δ 7.42 (m, 5H), 7.20 (d, J=8.4 Hz, 2H), 6.96 (d, J=8.4 Hz, 2H), 5.06 (s, 2H), 4.18 (s, 2H), 3.04 (s, 2H), 1.98 (s, 3H), 1.60-1.74 (m, 6H), 1.43 (s, 6H). MS: 418 (M+1). HPLC purity=86.3%.

### Synthesis of Compound 7

Synthesis of Compound 7 was carried out in a similar fashion to the above preparation using the hydroxy amide 107 (60 mg, 29%). ¹H NMR (300 MHz, CDCl₃): δ 7.47-7.32 (m, 5H), 7.16 (d, 2H, J=4.5 Hz), 6.94 (d, 2H, J=4.5 Hz), 6.69 (s, 1H, NH), 5.06 (s, 2H), 4.14 (s, 2H), 2.12 (s, 3H), 2.03 (s, 6H), 1.17 (s, 6H). MS: 404 [M+1]. HPLC purity 98.6%.

### Example 2

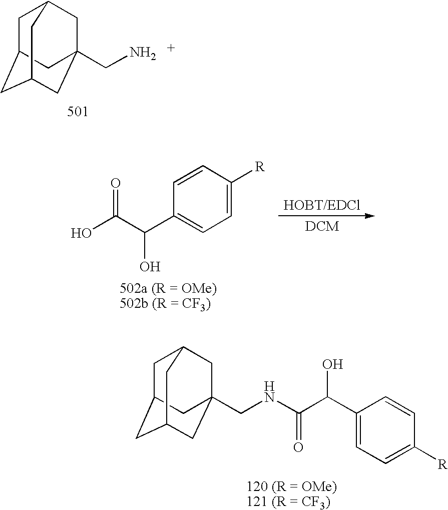

### Synthesis of Compound 120

To a solution of adamantylmethyl amine 501 (0.200 g, 1.21 mmol) in dichloromethane (5 mL) was added hydroxyl acid 502a (0.200 g, 1.09 mmol), hydroxybenzotriazole (0.175 g, 1.3 mmol), N-ethyl-N′-(3-dimethylaminopropyl)carbodiimide (0.249 g, 1.3 mmol) and diisopropylethylamine (0.258 g, 2.0 mmol), and the resulting mixture was stirred at room temperature overnight. The reaction mixture was then extracted with dichloromethane, and the organic extract was washed with saturated aqueous sodium bicarbonate solution (50 mL) followed by aqueous hydrochloric acid (10%, 50 mL). The residue obtained after drying the organic portion with sodium sulfate and removal of solvent was chromatographed on silica gel eluting with methanol (5%) in DCM to obtain the pure product 120 (0.21 g, 48%). ¹H NMR (CDCl₃, 300 MHz): δ 7.32 (d, J=8.1 Hz, 2H), 6.96 (d, J=8.1 Hz, 2H), 6.05 (bs, 1H), 5.00 (s, 2H), 3.82 (s, 3H), 2.96 (m, 2H), 1.95 (s, 3H), 1.55-1.72 (m, 6H), 1.39 (s, 6H). MS: 757 (2M+Na), 368 (M+1). HPLC purity=97.9%.

### Synthesis of Compound 121

Synthesis of Compound 121 was carried out in a similar fashion to the above preparation using hydroxyl acid 502b (0.20 g, 50%). ¹H NMR (CDCl₃, 300 MHz): δ 7.59 (m, 4H), 6.20 (bs, 2H), 5.13 (s, 2H), 2.96 (m, 2H), 1.95 (s, 3H), 1.55-1.72 (m, 6H), 1.38 (s, 6H). MS: 681 (2M+Na), 330 (M+1). HPLC purity=97.9%.

### Example 3

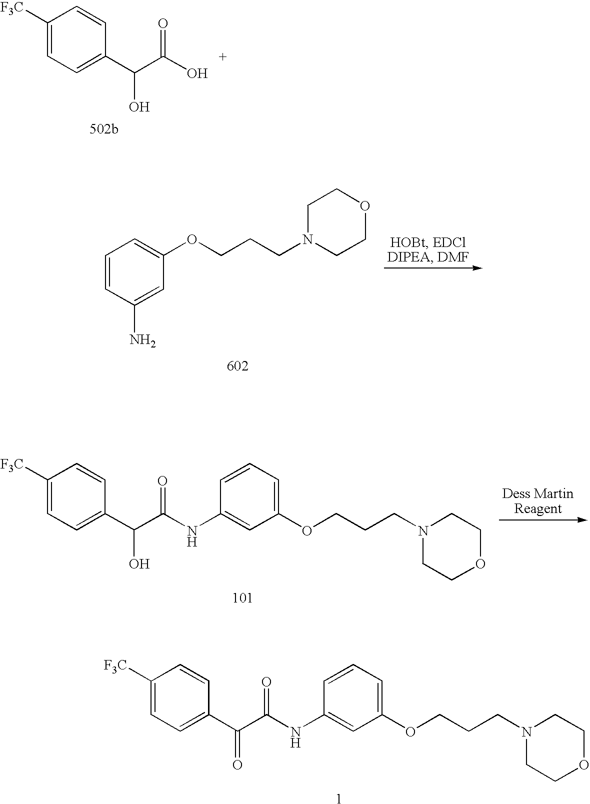

### Synthesis of Compound 101

To a stirred solution of mandelic acid 502b (200 mg, 0.907 mmol) in DMF (5 mL) was added Compound 602 (256 mg, 1.09 mmoL); HOBT (61 mg, 0.45 mmol), EDCI (519 mg, 2.72 mmol) and DIPEA (0.939 ml, 5.44 mmol) at 0° C. The reaction mixture stirred overnight at room temperature. Progress of the reaction was monitored by TLC. The reaction mixture was concentrated under vacuum to remove DMF, and the residue was dissolved in water and extracted with ethyl acetate. The organic layer washed with brine solution and dried over anhydrous sodium sulfate to give Compound 101 (20 mg, 5%) with HPLC purity 98%. Mass: 439 [M+1]. ¹H NMR (200 MHz; CDCl₃) δ: 2.0 (t, CH₂); 2.5 (m, 3×—CH₂); 3.78 (t, 2×—CH₂); 4.0 (t, CH₂); 5.3 (s, 1H); 6.7 (dd, 1H); 6.98 (dd, 1H); 7.2 (d, 2H); 7.38 (m, 2H); 7.65 (s, 2H); 8.25 (1H, NH).

### Synthesis of Compound 1

To a stirred solution of Compound 101 (60 mg, 0.14) in dry DCM (5 mL) was added Dess-Martin reagent (121 mg, 0.287 mmol) at room temperature. The reaction mixture was stirred for 4 hours at room temperature. Progress of the reaction was monitored by TLC. The reaction was quenched with a mixture of saturated aqueous sodium thiosulphate solution and saturated bicarbonate solution. The mixture was extracted with DCM, and the organic layer washed with brine solution and dried over anhydrous sodium sulfate to give Compound 1 (40 mg, 68%) with HPLC purity 88.7%. Mass: 437 [M+1]. ¹H NMR (200 MHz; CDCl₃) δ: 2.1 (m, —CH₂); 2.65 (m, 3×—CH₂); 3.8 (t, 2×—CH₂); 4.1 (t, —CH₂); 6.78 (dd, 1H); 7.3 (m, 2H); 7.45 (t, 1H); 7.8 (d, 2H); 8.58 (d, 2H); 8.95 (1H, NH).

### Example 4a

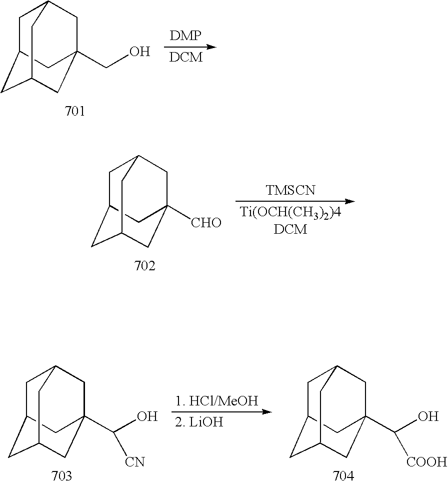

### Synthesis of Compound 704

Adamantyl-methanol 701 (5.0 g, 30 mmol) was dissolved in 40 mL of dry dichloromethane and to it was added portion-wise Dess-Martin Periodinate (13 g, 32 mmol) at 0° C. The resulting mixture was allowed to stir at room temperature for 1 h. After completion of the reaction, the reaction mixture was poured into water, and the organic layer was separated, washed with sodium bisulfite and brine, and dried over sodium sulfate. Evaporation of the organic layer provided 4.3 g of the corresponding aldehyde 702.

The aldehyde 702 (4.00 g, 24.3 mmol) was then dissolved in 30 mL of dry dichloromethane and to it was added Ti(OiPr)₄ (4.21 mL, 12.2 mmol) at 0° C., and the resulting mixture was stirred for 15 minutes at room temperature. Trimethylsilyl cyanide (TMSCN) (18.6 mL, 125 mmol) was added slowly, and the reaction stirred at room temperature for 6 h. After completion, a mixture of 1.5 N HCl and THF (1:1, 30 mL each) was added to the reaction mixture and stirred for 10 min. Solvent was removed under reduced pressure, and the mixture was extracted with ethyl acetate (3×50 mL). The organic layer was dried over Na₂SO₄, and concentrated to afford Compound 703 (3.4 g). ¹H NMR (200 MHz, CDCl₃): δ 4.26 (d, 1H); 2.43 (bs, 1H); 2.10-1.60 (m, 15H). IR cm⁻¹ (KBr) 3250, 2120.

To a solution of methanolic HCl (30 mL), cyanohydrin compound 703 (3.40 g, 17.9 mmol) was added, and the reaction mixture was refluxed for 6 h. Upon completion the reaction mixture was concentrated under reduced pressure. The residue was taken up in ethyl acetate and washed with water and brine solution and dried over anhydrous sodium sulfate. The organic layer was evaporated to dryness. The crude product was then stirred in 2 N LiOH solution (20 mL) for 2 h and then acidified with 4 N aqueous HCl to pH 5.0 and then extracted with 10% methanol in dichloromethane. The crude acid 704 was purified by column chromatography on silica gel (100-200 mesh) eluting with ethyl acetate:methanol:hexane (3:1:6) to afford Compound 704 (2.6 g). ¹HNMR (200 MHz, CDCl₃): δ 4.20 (d, 1H,); 2.85 (bs, 1H); 2.10-1.60 (m, 15H). IR cm⁻¹ (KBr) 3250, 1700.

### Example 4b

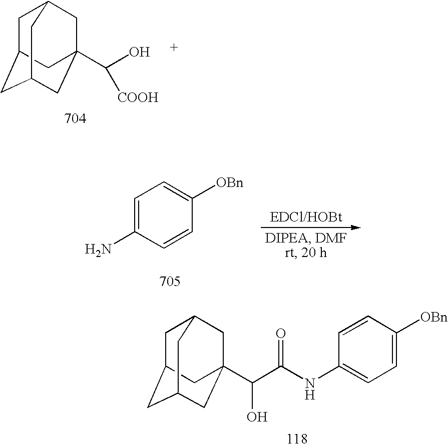

### Synthesis of Compound 118

Adamantyl-α-hydroxy acetic acid 704 (250 mg, 1.19 mmol) was dissolved in 15 mL of dry DMF and to it was added HOBT (187 mg, 1.38 mmol), and DIPEA (410 mg, 3.17 mmol), and the reaction mixture was stirred at room temperature for 5 minutes. 4-Benzyloxyaniline 705 (200 mg, 1.05 mmol) and EDCI (418 mg, 2.18 mmol) were then added, and the resulting mixture allowed to stir at room temperature for 20 h. After completion the reaction mixture was poured into water and extracted with dichloromethane (2×50 mL). The combined organic layers were then washed several times with water and brine, dried over anhydrous sodium sulfate and the solvent evaporated. The crude product was purified by column chromatography on silica gel (100-200 mesh) eluting with ethyl acetate:methanol:hexane (3:1:6) to afford Compound 118 as a white solid. (95 mg). ¹H NMR (200 MHz, CDCl₃): δ 8.0 (bs, 1H); 7.55-7.30 (m, 7H); 6.95 (m, 2H); 5.20 (s, 2H), 3.80 (s, 1H,); 2.65 (bs, 1H,); 2.10-1.60 (15H, m). Mass: 392 [M+1, 100%]. m. p. 186-188° C.

### Example 4c

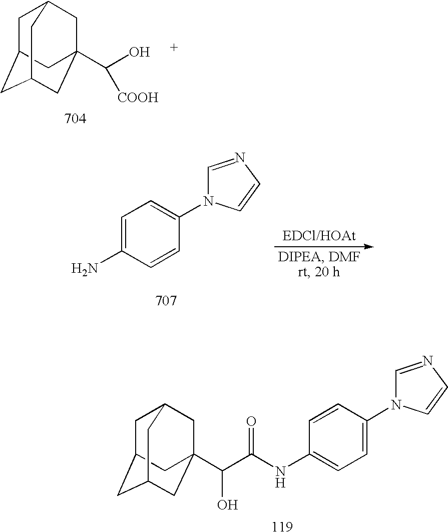

### Synthesis of Compound 119

Adamantyl-α-hydroxy acetic acid 704 (200 mg, 0.950 mmol) was dissolved in 15 mL of dry DMF and to it was added 1-hydroxy-7-azabenzotriazole (HOAT) (182 mg, 1.38 mmol) and DIPEA (400 mg, 3.27 mmol), and the reaction mixture were stirred at room temperature for 5 minutes. 4-Imidazolyl aniline 707 (160 mg, 1.00 mmol) and EDCI (418 mg, 2.28 mmol) were then added, and the mixture allowed to stir at room temperature for 20 h. After completion, the reaction mixture was poured into water and extracted with dichloromethane (2×50 mL). The organic layer was then washed several times with water and brine, dried over anhydrous sodium sulfate and evaporated. The crude product was purified by column chromatography on silica gel (100-200 mesh) eluting with ethyl acetate:methanol:hexane (3:1:6) to afford Compound 119 as a white solid. (85 mg). ¹H NMR (200 MHz, CDCl₃): δ 8.20 (bs, 1H); 7.80 (s, 1H), 7.65 (d, 1H), 7.25-7.10 (m, 5H), 4.20 (s, 1H,), 3.85 (bs, 1H,); 2.10-1.60 (15H, m). Mass: 352 [M+1, 100%]. m. p. 219-220° C.

### Example 4d

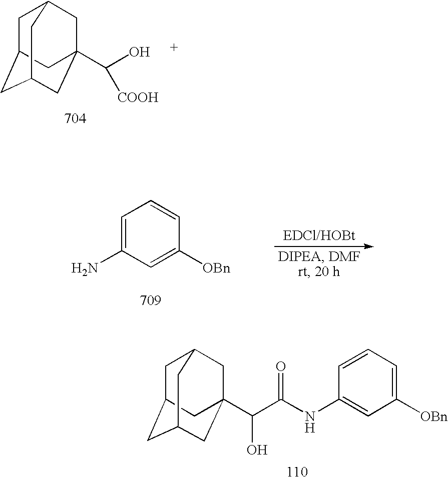

### Synthesis of Compound 110

Adamantyl-α-hydroxy acetic acid 704 (250 mg, 1.19 mmol) was dissolved in 15 mL of dry DMF and to it was added HOBT (187 mg, 1.38 mmol), DIPEA (410 mg, 3.17 mmol), and the reaction mixture was stirred at room temperature for 5 minutes. 3-Benzyloxyaniline 709 (200.mg 1010.mmol) and EDCI (418 mg, 2.18 mmol) were then added, and the mixture allowed to stir at room temperature for 20 h. After completion the reaction mixture was poured into water and extracted with dichloromethane (2×50 mL). The organic layer was then washed several times with water and brine, dried over anhydrous sodium sulfate and evaporated. The crude product was purified by column chromatography on silica gel (100-200 mesh) eluting with ethyl acetate:methanol:hexane (3:1:6) to afford Compound 110 as a white solid. (145 mg). ¹HNMR (200 MHz, CDCl₃): δ 8.0 (bs, 1H); 7.55-7.30 (m, 7H); 7.00 (d, 1H); 6.75 (d, 1H); 5.10 (s, 2H), 3.80 (s, 1H); 2.65 (bs, 1H,); 2.10-1.60 (15H m). Mass: 392 [M+1, 100%]. m. p. 130-132° C.

### Example 4e

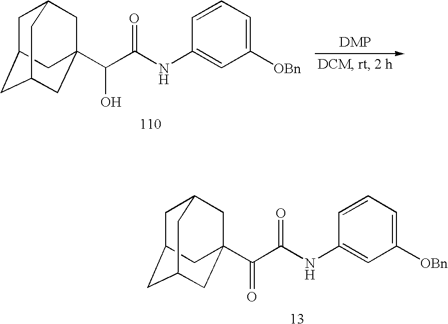

### Synthesis of Compound 13

Compound 110 (130 mg, 0.30 mmol) was dissolved in 10 mL of dry dichloromethane and to it was added portion-wise Dess-Martin periodinane (170 mg, 0.39 mmol) at 0° C. and allowed to stir at room temperature for 2 h. After completion the reaction mixture was poured into water and the organic layer was separated, washed with sodium bisulfite and brine and dried over sodium sulfate. Evaporation of the organic layer provided 120 mg of crude product which was purified by column chromatography on silica gel (100-200 mesh) eluting with ethyl acetate:methanol:hexane (3:1:6) to afford Compound 13 (105 mg) as a white solid. (¹H NMR (200 MHz, CDCl₃): δ 8.60 (bs, 1H); 7.55-7.30 (m, 7H); 7.00 (d, 1H); 6.75 (d, 1H); 5.10 (s, 2H), 2.10-1.60 (15H, m). Mass: 390 [M+1, 100%]. m. p. 106-107° C.

### Example 5

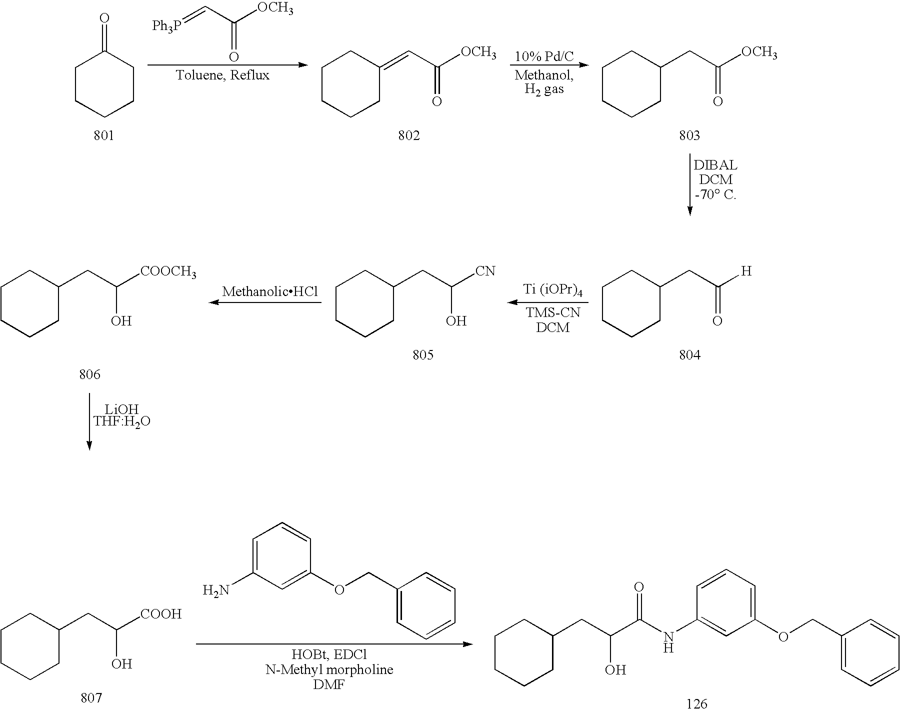

### Synthesis of Compound 802

To a stirred solution of cyclohexanone 801 (15.0 g, 153 mmol) in toluene (100 mL) was added methoxycarbonylmethylene triphenylphosphorane (52.8 g, 153 mmol). The reaction mixture was refluxed overnight. After completion of the reaction, the reaction mixture was diluted with ethyl acetate and washed with water and brine and dried over anhydrous sodium sulfate to afford product cyclohexylidene-acetic acid methyl ester 802 (5.0 g, 21%). ¹H NMR (200 MHz, CDCl₃): δ 1.6-2.0 (m, 10H), 3.7 (s, 3H), 5.6 (s, 1H).

### Synthesis of Compound 803

To a stirred solution of cyclohexylidene-acetic acid methyl ester 802 (5.00 g, 32.5 mmol) in methanol (100 mL) was added 10% Pd/C (1.5 gm) under a nitrogen atmosphere. The reaction mixture was stirred under a H₂ atmosphere for 3 hrs. After completion of the reaction, the reaction mixture was filtered through a bed of celite, and the filtrate was concentrated to obtain (3.8 g, 75%) cyclohexyl-acetic acid methyl ester 803. ¹H NMR (200 MHz, CDCl₃): δ 1.0-2.0 (m, 11H), 2.2 (d, 2H), 3.7 (s, 3H).

### Synthesis of Compound 804

To a stirred solution of cyclohexyl-acetic acid methyl ester 803 (3.80 g, 24.4 mmol) in DCM (10 mL) was added DIBAL (diisobutylaluminium hydride) (7 mL, 1.6 M solution) at −70° C. The reaction mixture stirred at −70° C. for 5 hrs. After completion of the reaction, the reaction mixture was quenched with saturated aqueous ammonium chloride and further diluted with DCM (10 mL), washed with water and brine and dried over anhydrous sodium sulfate to obtain cyclohexylacetaldehyde 804 (3.0 g, 97%). Mass: 126 [M+H]; ¹H NMR (200 MHz, CDCl₃): δ 1.0-2.0 (m, 11H), 2.3 (dd, 2H), 9.8 (s, 1H).

### Synthesis of Compound 805

To a stirred solution of cyclohexylacetaldehyde 804 (3.00 g, 23.8 mmol) in DCM (30 mL) was added titanium isopropoxide (2.8 mL, 9.5 mmol) at 0° C. After raising the reaction mixture to room temperature, trimethysilyl cyanide (11.1 mL, 83.3 mM) was added and the mixture was stirred at room temperature for 4 hrs. After completion, the reaction mixture was quenched with 1.5 N HCl (10 mL) and THF (10 mL) at 0° C. and extracted with ethyl acetate (50 mL). The organic portion was washed with water and brine and dried over anhydrous sodium sulfate to obtain 3-cyclohexyl-2-hydroxy-propionitrile 805 (3.0 g, 55%). Mass: 154 [M+H]; ¹H NMR (200 MHz, CDCl₃): δ 1.0-2.0 (m, 11H), 2.3 (m, 2H), 4.6 (m, 1H).

### Synthesis of Compound 806

To 3-Cyclohexyl-2-hydroxy-propionitrile 805 (2.0 g, 13.1 mmol) was added methanolic HCl (30 mL), and the reaction mixture was heated to 70-80° C. for 4 hrs. After completion, the reaction mixture was concentrated under a vacuum to remove the solvent, the residue was extracted with ethyl acetate (2×50 mL), and the organic portion was washed with water, and brine then dried over anhydrous sodium sulfate to obtain 3-cyclohexyl-2-hydroxy-propionic acid methyl ester 806 (1.5 g, 61%). Mass: 187 [M+H]; ¹H NMR (200 MHz, CDCl₃): δ 0.9-2.0 (m, 13H), 3.8 (s, 3H), 4.3 (d, 1H).

### Synthesis of Compound 807

To a stirred solution of cyclohexyl-hydroxy-acetic acid methyl ester 806 (1.50 g, 8.06 mmol) in THF (10 mL) and H₂O (10 mL) was added lithium hydroxide (719 mg, 16.9 mmol) at 0° C., and the mixture was stirred at room temperature for 4 hrs. After completion, the reaction mixture was concentrated under vacuum to remove THF, and the residue obtained was adjusted to pH 2 at 0° C. and extracted with ethyl acetate (2×50 mL). The combined ethyl acetate layers were washed with water and brine and dried over anhydrous sodium sulfate to obtain 3-cyclohexyl-2-hydroxy-propionic acid 807 (1.3 g, 72%). ¹H NMR (200 MHz, CDCl₃): δ 0.9-2.0 (m, 11H), 2.2 (d, 2H), 4.3 (d, 1H).

### Synthesis of Compound 126

To a stirred solution of 3-cyclohexyl-2-hydroxy-propionic acid 807 (300 mg, 1.74 mmol) in DMF (10 mL) was added 3-benzyloxy-phenylamine (243 mg, 1.22 mmol), hydroxybenzotriazole (117 mg, 0.87 mmol), N-(3-dimethylaminopropyl)-N′-ethylcarbodiimide (499 mg, 2.61 mmol), and N-methylmorpholine (0.50 mL, 5.23 mmol) at 0° C. The reaction mixture was stirred at room temperature overnight. After completion, the reaction mixture was concentrated under vacuum to remove DMF completely. The residue obtained was dissolved in water and extracted with ethyl acetate (2×50 mL), and the organic layer was washed with brine and dried over anhydrous sodium sulfate. The solid obtained on removal of solvent was purified by column chromatography on silica gel to obtain N-(3-benzyloxy-phenyl)-3-cyclohexyl-2-hydroxy-propionamide 126 (10 mg, 1.6%). Mass: 354 [M+H]; ¹H NMR (500 MHz, CDCl₃): 0.99-1.46 (m, 5H), 1.88 (m, 6H), 1.9 (m, 2H), 4.38 (m, 1H), 5.1 (s, 2H), 6.78 (m, 1H), 7.1 (d, 1H), 7.2-7.6 (m, 7H).

### Example 6

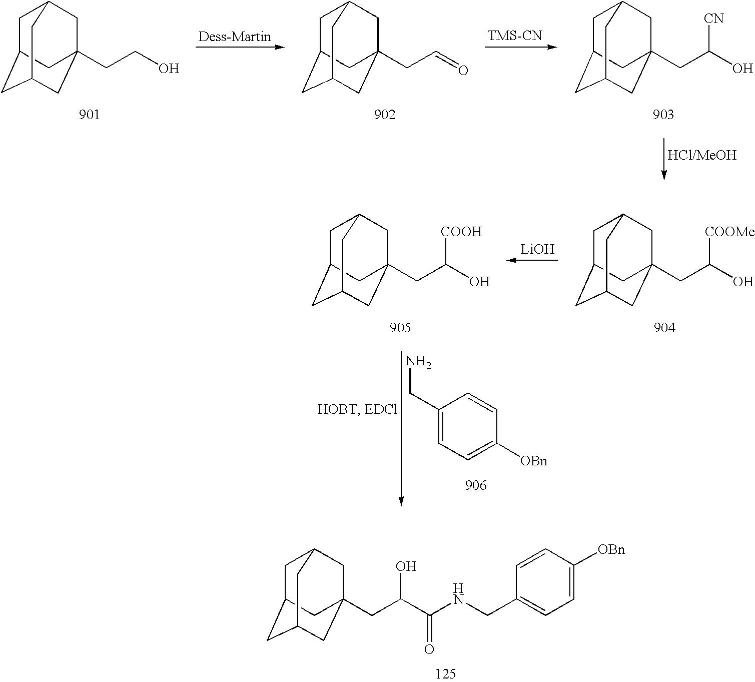

### Synthesis of Compound 902

To a solution of compound 901 (2.00 g, 11.1 mmol) in dichloromethane (30 mL) was added Dess-Martin periodinane (5.59 g, 153 mmol), and the mixture was stirred at room temperature for 4 hrs. After completion of the reaction, the reaction mixture was diluted with DCM, washed with water followed by brine and dried over anhydrous sodium sulfate. The solvent was removed under reduced pressure to afford compound 902 (1.8 g, 91%). ¹H NMR (200 MHz, CDCl₃): δ 9.6 (s, 1H), 2.57 (s, 2H), 1.6-2.0 (m, 15H).

### Synthesis of Compound 903

To a stirred solution of compound 902 (1.80 g, 10.1 mmol) in DCM (20 mL) was added titanium isopropoxide (1.42 g, 4.92 mmol) at 0° C. The reaction mixture was raised to room temperature, and trimethylsilyl cyanide (3.47 g, 35.0 mmol) was added and stirred at room temperature for 4 hrs. After completion, the reaction mixture was quenched with an equimolar mixture of 1.5 N HCl (10 mL) and THF (10 mL) at 0° C. and extracted with ethyl acetate (2×50 mL). The organic layer was washed with water and brine and dried over anhydrous sodium sulfate. The solvent was removed under the reduced pressure, and the resulting crude product was purified by column chromatography on silica gel to obtain compound 903 (1.7 g, 82%). Mass: 205 [M+H]; ¹H NMR (200 MHz, CDCl₃): δ 4.4 (m, 1H), 2.1-1.4 (m, 17H).

### Synthesis of Compound 904

To compound 903 (1.70 g, 8.29 mmol) was added methanolic HCl (25 mL), and the reaction mixture was heated to 70-80° C. for 4 hrs. After completion, the reaction mixture was concentrated under vacuum to remove the solvent. The residue was extracted with ethyl acetate (2×50 mL) and the organic layer washed with water and brine and dried over anhydrous sodium sulfate. The solvent was removed under reduced pressure to provide compound 904 (1.2 g, 61%). Mass: 238 [M+H]; ¹H NMR (200 MHz, CDCl₃): δ 4.4 (m, 1H), 3.8 (s, 3H), 2.1-1.4 (m, 17H).

### Synthesis of Compound 905

To a stirred solution of compound 904 (1.20 g, 5.05 mmol) in THF (10 mL) and H₂O (10 mL) was added lithium hydroxide (528 mg, 12.6 mmol) at 0° C., and the reaction mixture was stirred at room temperature for 4 hrs. After completion, the reaction mixture was concentrated under vacuum to remove THF, and the residue obtained was adjusted to pH 2 at 0° C. and extracted with ethyl acetate (2×50 mL). The combined organic layers were washed with water and brine and dried over anhydrous sodium sulfate. The solvent was removed under reduced pressure to obtain compound 905 (800 mg, 71%). Mass: 223 [M+H]; ¹H NMR (200 MHz, CDCl₃): δ 4.4 (m, 1H), 2.1-1.4 (m, 17H).

### Synthesis of Compound 125

To a stirred solution of compound 905 (200 mg, 0.890 mmol) in DMF (10 mL) was added 4-benzyloxy-phenylamine 906 (228 mg, 1.07 mmol); HOBT (303 mg, 2.23 mmol); EDCI (427 mg, 2.23 mmol) and N-methylmorpholine (270 mg, 2.67 mmol) at 0° C. The reaction mixture was stirred at room temperature overnight. After completion of the reaction, the reaction mixture was concentrated under vacuum to remove DMF completely. The residue obtained was dissolved in water and extracted with ethyl acetate (2×50 mL) and the organic layer washed with brine and dried over anhydrous sodium sulfate. The solvent was evaporated to obtain crude product which was purified by column chromatography to obtain 125 (92 mg). Mass: 420 [M+H]; ¹H NMR (500 MHz, CDCl₃): δ 0.99-1.4 (m, 10H), 1.88 (m, 6H), 1.9 (m, 2H), 4.38 (m, 1H), 4.41 (s, 2H), 5.1 (s, 2H); 6.78 (m, 1H), 6.9 (d, J=10 Hz, 2H), 7.2-7.6 (m, 7H).

### Example 7

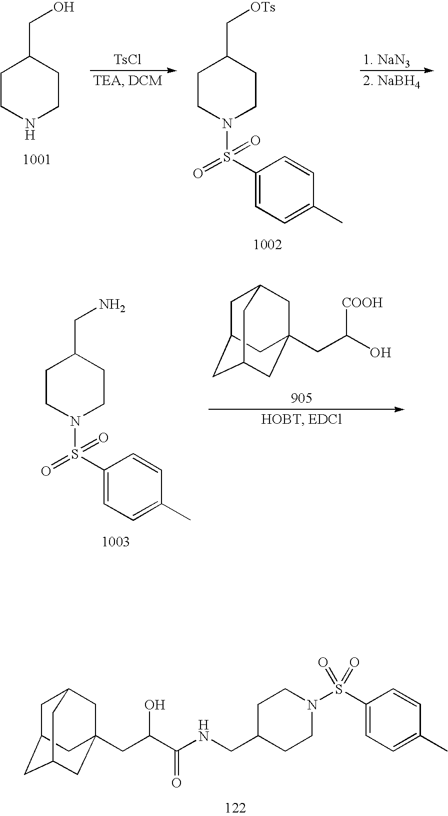

### Synthesis of Compound 1003

Piperidine-4-methanol 1001 (3.0 g, 26 mmol) was dissolved in dichloromethane (100 mL) followed by addition of TEA (2.63 mL, 26 mmol) at 0° C. 4-Methylbenzenesulfonyl chloride (12.4 g, 65.0 mmol) was then added dropwise. The reaction mixture was stirred at room temperature for 2 hrs. After completion of reaction, the mixture was poured into water (100 mL) and extracted with dichloromethane (100 mL). The organic layer was washed with brine, dried over anhydrous sodium sulfate and solvent removed to afford 1002 (5.0 g). The crude product 1002 was then taken in DMF:water (30 mL, 9:1), to which sodium azide (2.3 g) was added and heated at 80° C. for 8 hrs. After completion of the reaction, the mixture was poured into water (100 mL) and extracted with dichloromethane, dried over sodium sulfate and solvent evaporated under reduced pressure. The crude product obtained was then taken in water (50 mL) and sodium borohydride (2.0 g) and cobalt (II) chloride (1.0 g) was added and stirred at room temperature for 20 minutes. After completion, the reaction mixture was filtered through celite, and the crude product 1003 obtained after evaporation was purified by column chromatography on silica gel eluting with methanol:DCM (1:9) to obtain 1003 (2.5 g). ¹H NMR (200 MHz, CDCl₃): δ 7.80 (m, 2H), 7.40 (m, 2H), 2.8-2.7 (m, 4H), 2.63 (m, 1H), 2.53 (d, 2H), 2.20 (bs, 2H), 1.8-1.6 (m, 4H). Mass: 323 (M+H).

### Synthesis of Compound 122

To a stirred solution of compound 905 (150 mg, 0.660 mmol) in DMF (10 mL) was added compound 1003 (212 mg, 0.790 mmol); HOBT (224 mg, 1.65 mmol); EDCI (315 mg, 1.64 mmol) and N-methylmorpholine (215 mg, 3.12 mmol) at 0° C. The reaction mixture was stirred at room temperature overnight. After completion, the reaction mixture was concentrated under vacuum to remove DMF completely. The residue was poured into water, and the mixture was extracted with ethyl acetate (2×50 mL). The organic layers were washed with brine and dried over anhydrous sodium sulfate. The solvent was evaporated, and the residue was purified by column chromatography on silica gel to afford 122 (95 mg) with an HPLC purity of 96%. Mass: 475 [M+H]; ¹H NMR (500 MHz, CDCl₃): δ 1.22-1.60 (m, 18H), 1.9 (m, 4H), 2.25 (m, 2H), 2.43 (s, 3H), 3.17 (m, 2H), 3.78 (m, 2H), 4.26 (m, 1H); 6.63 (m, 1H), 7.36 (d, J=10 Hz, 2H), 7.65 (d, J=10 Hz, 2H).

### Example 8

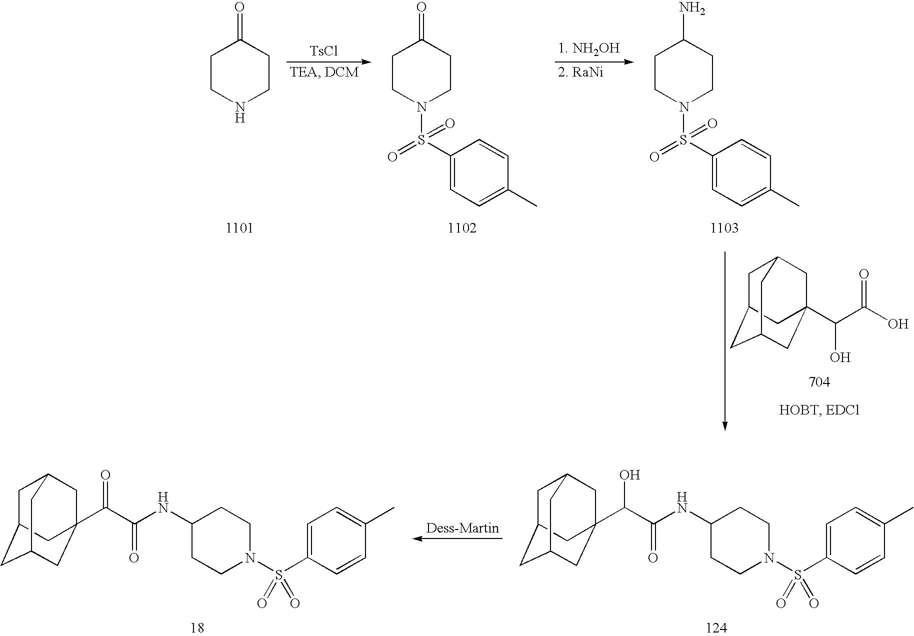

### Synthesis of Compound 1103

4-Piperidone hydrochloride 1101 (5.0 g, 37 mmol) was dissolved in dichloromethane (100 mL) and to it was added TEA (9.3 mL, 92 mmol) at 0° C. followed by dropwise addition of 4-methylbenzenesulfonyl chloride (8.44 g, 44 mmol). The reaction mixture was then stirred at room temperature for 2 hrs. The mixture was then poured into water (100 mL) and extracted with dichloromethane. The organic layer was then washed with brine, dried over anhydrous sodium sulfate, and the solvent removed to afford 1102. The crude product 1102 was then taken in ethanol (50 mL) and hydroxylamine hydrochloride (4.1 g, 52 mmol) added followed by pyridine (3.6 mL, 52 mmol). The reaction mixture was then heated at 65° C. for 2 hours and then the alcohol was evaporated off under a vacuum. The crude product obtained was then taken in methanol (50 mL), Raney Ni (RaNi) (2 g) added, and heated at 50° C. for 3 hours. After completion, the reaction mixture was filtered through celite, and the solvent was evaporated. The crude product obtained was purified by column chromatography on silica gel eluting with methanol:DCM (1:9) to obtain 1103 (4.5 g). ¹H NMR (200 MHz, CDCl₃): δ 7.83 (M, 2H), 7.37 (m, 2H), 2.8-2.7 (m, 4H), 2.63 (m, 1H), 2.2 (bs, 2H), 1.8-1.6 (m, 4H). Mass: 309 [M+H].

### Synthesis of Compound 124

To a stirred solution of compound 704 (150 mg, 0.710 mM) in DMF (10 mL) was added compound 1103 (216 mg, 0.85 mmol); HOBT (243 mg, 1.78 mmol); EDCI (341 mg, 1.77 mmol) and N-methylmorpholine (215 mg, 2.12 mmol) at 0° C. The reaction mixture was stirred at room temperature overnight. After completion, the reaction mixture was concentrated under vacuum to remove DMF completely. The residue obtained was then dissolved in water and extracted with ethyl acetate (2×50 mL), and the organic layer washed with brine and dried over anhydrous sodium sulfate. The solvent was evaporated, and the crude product obtained was purified by column chromatography on silica gel to afford compound 124 (50 mg) with HPLC purity of 91%. Mass: 447 [M+H]; ¹H NMR (500 MHz, CDCl₃): δ 1.55-1.70 (m, 15H), 1.9 (m, 4H); 2.41 (m, 2H), 2.43 (s, 3H), 3.45 (m, 1H), 3.78 (m, 2H), 6.03 (m, 1H), 7.36 (d, J=10 Hz, 2H), 7.61 (d, J=10 Hz, 2H).

### Synthesis of Compound 18

Compound 124 (55 mg, 0.123 mmol) was dissolved in dichloromethane (20 mL) and to it was added portionwise Dess-Martin periodinane (63 mg, 0.125 mmol) at 0° C. and allowed to stir at room temperature for 2 hrs. After completion, the reaction mixture was poured into water, and the organic layer was separated, washed with aq.sodium bisulfite and brine, and dried over sodium sulfate. The crude product obtained on evaporation of solvent was purified by column chromatography on silica gel eluting with ethyl acetate:methanol:hexane (3:1:6) to obtain the compound 18 (45 mg) as a white solid. ¹H NMR (200 MHz, CDCl₃): δ 7.65 (d, 2H), 7.40 (d, 2H), 6.78 (bs, 1H), 3.65 (m, 1H), 2.40 (s, 3H), 2.10-1.90 (m, 4H0, 1.80-1.70 (m, 4H,), 1.70-1.60 (m, 15H). Mass: 445 [M+H].

### Example 9

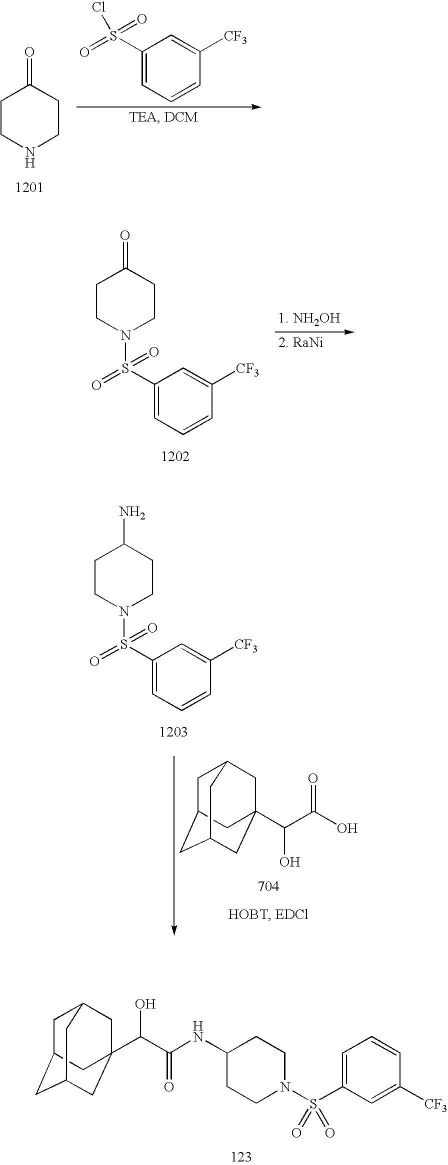

### Synthesis of Compound 1203

4-Piperidone hydrochloride 1201 (5.0 g, 37 mmol) was dissolved in dichloromethane (100 mL) and to it was added TEA (9.3 mL, 92.2 mmol) at 0° C. followed by dropwise addition of 3-trifluoromethylbenzenesulfonyl chloride (10.8 g, 44.0 mmol). The reaction mixture was then stirred at room temperature for 2 hrs. After completion of reaction, it was poured into water (100 mL) and extracted with dichloromethane. The organic layer was then washed with brine, dried over anhydrous sodium sulfate and evaporated to gave 8 g of 1202. Crude 1202 was then taken in ethanol (50 mL) and hydroxylamine hydrochloride (4.1 g, 52 mmol) followed by pyridine (3.6 mL, 52 mmol) was added. The reaction mixture was then heated at 65° C. for 2 hours and then the alcohol then evaporated under a vacuum. The crude residue was then taken in 50 mL of methanol and to it added Raney Ni (2 g) and heated at 50° C. for 3 hours. After completion of reduction, the reaction mixture was filtered through celite, and the crude product was purified by chromatography on silica gel (100-200 mesh) eluting with methanol:DCM (1:9) to give 4.5 g 1203. ¹H NMR (200 MHz, CDCl₃): δ 8.12 (s, 1H); 7.93 (d, 1H); 7.47-7.40 (m, 2H); 2.8-2.7 (m, 4H); 2.63 (m, 1H); 2.2 (bs, 2H); 1.8-1.6 (m, 4H). Mass: 309 (M+1, 100%).

### Synthesis of Compound 123

To a stirred solution of compound 1203 (125 mg, 0.590 mM) in DMF (10 mL) was added compound 704 (218 mg, 0.710 mM), HOBT (202 mg, 1.48 mM), EDCI (282 mg, 1.48 Mm), and N-methylmorpholine (279 mg, 1.76 mM) at 0° C. The reaction mixture was stirred at room temperature overnight. The progress of the reaction was monitored by TLC. After completion of the reaction, the reaction mixture concentrated under vacuum to remove DMF completely. The residue was dissolved in water and extracted with ethyl acetate (2 times), and the organic layer was washed with brine solution and dried over anhydrous sodium sulfate. The solvent was evaporated, and the residue was purified by column chromatography to afford compound 123 (50 mg) with HPLC purity of 90%. Mass: 501 [M+]; ¹H NMR (500 MHz, CDCl₃): δ 1.22-1.60 (m, 10H); 1.9 (m, 6H); 2.43 (m, 2H), 3.78 (m, 3H), 6.09 (m, 1H); 7.71 (m, 1H), 7.91 (m, 1H), 7.94 (m, 1H), 8.01 (m, 1H).

### Example 10

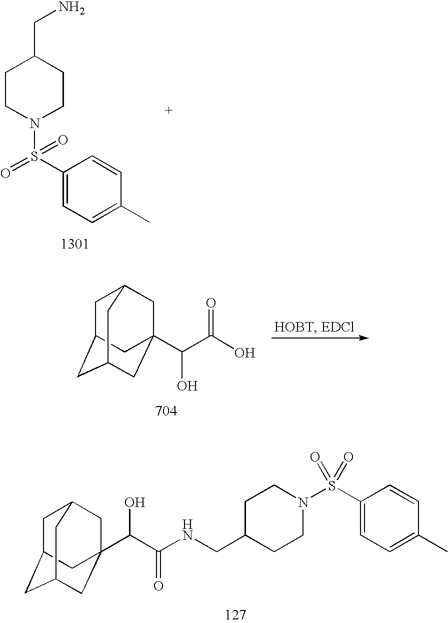

### Synthesis of Compound 127

To a stirred solution of compound 1301 (293 mg, 1.11 mmol) in DMF (10 mL) was added compound 704 (228 mg, 0.850 mmol), HOBT (243 mg, 1.73 mmol), EDCI (340 mg, 1.74 mmol) and N-methylmorpholine (215 mg, 2.12 mmol) at 0° C. The reaction mixture was stirred at room temperature overnight. After completion of the reaction, the reaction mixture was concentrated under vacuum. The residue was dissolved in water and extracted with ethyl acetate (2×50 mL), and the organic layer washed with brine and dried over anhydrous sodium sulfate. The solvent was evaporated to afford a residue which was purified by column chromatography on silica gel to obtain compound 127 (16 mg) with HPLC purity of 96%. Mass: 461 [M+H]; ¹H NMR (500 MHz, CDCl₃): δ 1.45-1.77 (m, 15H), 1.97 (m, 4H), 2.27 (m, 2H), 2.43 (s, 3H), 3.26 (m, 2H), 3.78 (m, 2H), 6.23 (m, 1H), 7.36 (d, J=10 Hz, 2H), 7.61 (d, J=10 Hz, 2H).

### Example 11

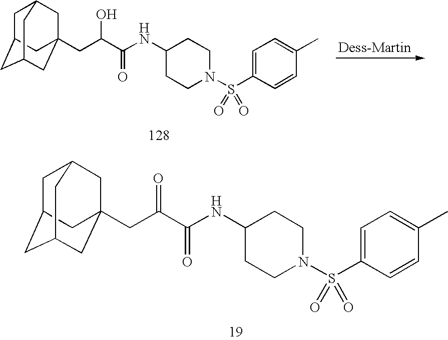

### Synthesis of Compound 19

Compound 128 (180 mg, 0.391 mmol) was dissolved in dry DCM (20 mL) and Dess-Martin periodinane (200 mg, 0.470 mmol) was added portionwise at 0° C., and the mixture allowed to stir at room temperature for 2 hrs. After completion of the reaction, the reaction mixture was poured into water and the organic layer was separated, washed with sodium bisulfite and brine, and dried over sodium sulfate. The crude product obtained on evaporation of solvent was purified by column chromatography on silica gel eluting with ethyl acetate:methanol:hexane (3:1:6) to obtain the compound 19 as white solid. (150 mg). ¹H NMR (200 MHz, CDCl₃): δ 7.65 (d, 2H), 7.40 (d, 2H), 6.70 (bs, 1H), 3.65 (m, 1H), 2.70 (s, 2H), 2.40 (s, 3H), 2.10-1.90 (m, 4H,), 1.80-1.70 (m, 4H,), 1.70-1.60 (m, 15H). Mass: 459 [M+1].

### Example 12

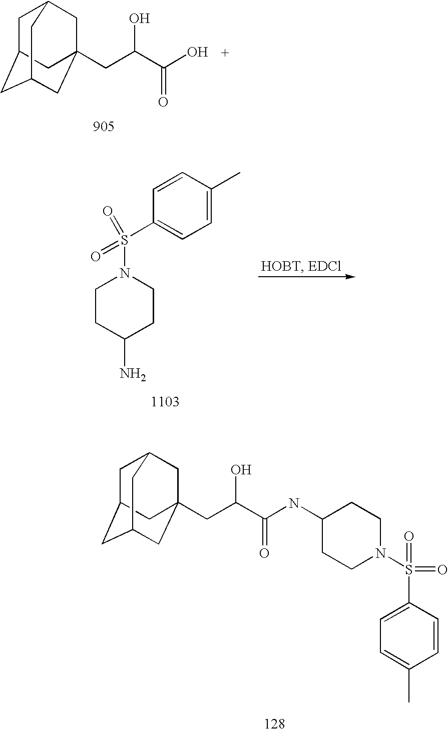

### Synthesis of Compound 128

To the stirred solution of compound 905 (200 mg, 0.89 mmol) in DMF (15 mL) was added compound 1103 (271 mg, 1.71 mmol), HOAT (303 mg, 2.32 mmol), EDCI (341 mg, 1.77 mmol) and N-methylmorpholine (279 mg, 2.26 mmol) at 0° C. The reaction mixture was stirred at room temperature overnight. The progress of the reaction mixture was monitored by TLC. After completion of the reaction, the reaction mixture was concentrated under vacuum to remove DMF completely. The residue obtained was dissolved in water, extracted with ethyl acetate (2×50 mL), and the combined organic layers were washed with brine and dried over anhydrous sodium sulfate. The solvent was evaporated and the residue was purified by column chromatography on silica gel eluting with ethyl acetate:methanol:hexane (3:1:6) to afford compound 128 (150 mg) as white solid. ¹H NMR (200 MHz, CDCl₃): δ 7.65 (d, J=10 Hz, 2H), 7.40 (d, J=10 Hz, 2H), 6.70 (bs, 1H), 5.75 (bs, 1H), 4.75 (m, 1H), 3.65 (m, 1H), 2.70 (m, 2H), 2.40 (s, 3H), 2.10-1.90 (m, 4H), 1.80-1.70 (m, 4H), 1.70-1.60 (m, 15H). Mass: 461 [M+1, 100%]. m. p. 178-180° C.

### Example 13

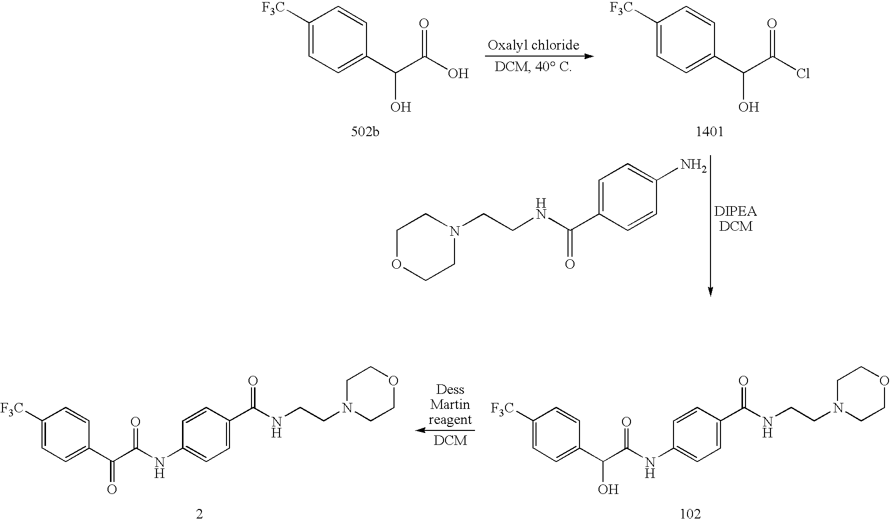

### Synthesis of Compound 1401

To a stirred solution of mandelic acid 502b (200 mg, 0.900 mmol) in DCM (5 mL) was added oxalyl chloride (0.56 mL, 6.0 mmol) at 0° C. The reaction mixture was refluxed for 3 hrs. After completion of the reaction, the reaction mixture was concentrated under vacuum to remove oxalyl chloride completely, and the residue was used directly for next reaction.

### Synthesis of Compound 102

To a stirred solution of compound 1401 (216 mg, 0.907 mmol) in DCM (5 mL) was added DIPEA (0.78 mL, 4.5 mmol) and amine compound (271 mg, 1.09 mmol) at 0° C. The reaction mixture stirred at room temperature overnight. The progress of the reaction mixture was monitored by TLC. After completion of the reaction, the reaction mixture concentrated under vacuum to remove DCM, the residue was dissolved in water and extracted with ethyl acetate (2×50 mL), and the organic layer was washed with brine and dried over anhydrous sodium sulfate to obtain coupled product which was purified by column chromatography to afford compound 102 (100 mg, 24.5%) with HPLC purity of 99.2%. Mass: 452 [M+H]; ¹H NMR (200 MHz, CDCl₃): δ 2.5 (m, 4H); 2.6 (m, 2H); 3.58 (m, 2H); 3.75 (m, 4H); 5.3 (s, 1H); 6.78 (m, 1H), 7.58-7.82 (m, 8H); 8.5 (bs, 1H).

### Synthesis of Compound 2

To a stirred solution of compound 102 (100 mg, 0.22 mmol) in DCM (5 mL) was added Dess-Martin reagent (197 mg, 0.460 mmol) at room temperature. The reaction mixture was stirred at room temperature for 3 hrs. The progress of the reaction was monitored by TLC. After completion of the reaction, the reaction mixture was quenched with a saturated solution of sodium thiosulphate and saturated sodium bicarbonate and extracted with DCM (2×50 mL). The organic layer was washed with brine and dried over anhydrous sodium sulfate to obtain coupled product which was purified by column chromatography to afford compound 2 (20 mg, 20%) with HPLC purity of 99.5%. Mass: 450 [M+H]. ¹H NMR (200 MHz, CDCl₃): δ 2.5 (m, 4H); 2.62 (m, 2H); 3.58 (m, 2H); 3.79 (m, 4H); 6.8 (bs, 1H); 7.77-7.85 (m, 6H); 8.56 (m, 2H); 9.1 (bs, 1H).

### Synthesis of Compound 1503

To compound 1502 (1.40 gm, 5.88 mmol) was added methanolic HCl (10 mL), and the reaction mixture heated to 70° C.-80° C. for 4 hrs. The progress of the reaction mixture was monitored by TLC. After completion, the reaction mixture was concentrated under vacuum to remove solvent, and the residue was extracted with ethyl acetate (2×50 mL). The organic layer was washed with water and brine and dried over anhydrous sodium sulfate to obtain crude product which was purified by column chromatography eluting with 15% ethyl acetate in hexanes to afford (800 mg, 53.3%) of compound 1503. Mass: 273, [M+H]; ¹HNMR (200 MHz, CDCl₃): δ 3.4 (d, 1H), 3.8 (s, 3H), 5.1 (s, 2H), 5.2 (d, 1H), 6.9-7.6 (m, 9H).

### Synthesis of Compound 1504

To a stirred solution of compound 1503 (800 mg, 2.94 mmol) in THF (20 mL) and H₂O (20 mL) was added lithium hydroxide (616 mg, 14.7 mmol) at 0° C. The reaction mixture was stirred at room temperature for 4 hrs. The progress of the reaction mixture was monitored by TLC. After completion, the reaction mixture was concentrated under vacuum to remove THF. The residue obtained was adjusted to pH 2 at 0° C. and extracted with ethyl acetate (2×50 mL). The organic layer was washed with water and brine and dried over anhydrous sodium sulfate to afford 1504 (700 mg, 83%). ¹H NMR (200 MHz, CDCl₃): δ 4.98 (s, 1H), 5.08 (s, 2H), 6.8-7.6 (m, 9H).

### Synthesis of Compound 103

To a stirred solution of compound 1504 (500 mg, 1.93 mmol) in DMF (10 mL) was added adamantyl-amine hydrochloride (436 mg, 2.32 mmol), HOBT (131 mg, 0.96 mmol), EDCI (1.10 gm, 5.81 mmol), and DIPEA (2.0 mL, 11.6 mmol) at 0° C. The reaction mixture was stirred at room temperature overnight. After completion of the reaction, the reaction mixture was concentrated under vacuum to remove DMF completely. The residue was dissolved in water and extracted with ethyl acetate (2×50 mL), and the organic layer washed with brine and dried over anhydrous sodium sulfate to afford the coupled product (256 mg, 96.6%) which was purified by preparative column chromatography to obtain compound 103 with HPLC purity of 96.5%. Mass: 392 [M+H]; ¹H NMR (200 MHz, CDCl₃): δ 1.5-2.2 (m, 15H), 3.79 (d, 1H), 4.82 (m, 1H), 5.1 (s, 2H), 5.6 (bs, 1H), 6.85-7.5 (m, 9H).

### Example 14

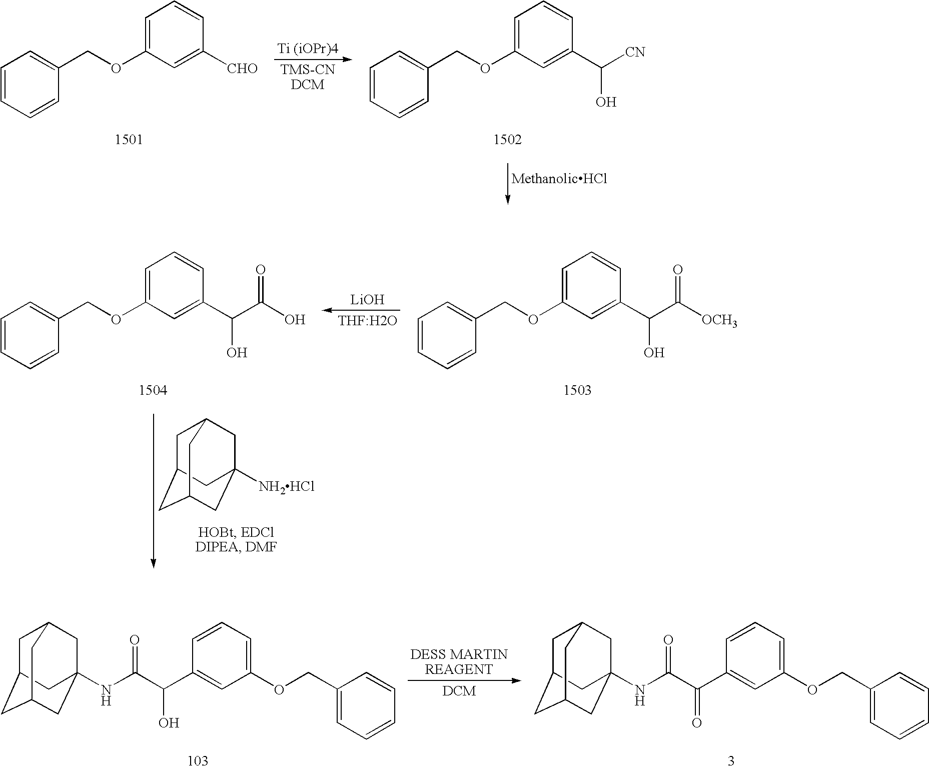

### Synthesis of Compound 1502

To a stirred solution of compound 1501 (1.30 gm, 5.85 mmol) in DCM (30 mL) was added Ti(iOPr)₄ (0.69 mL, 2.34 mmol) at 0° C. The reaction mixture was maintained at room temperature and TMSCN (3.1 mL, 23.4 mmol) was added. The reaction mixture was stirred at room temperature for 4 hrs. The progress of the reaction mixture was monitored by TLC. After completion, the reaction mixture was quenched with 1.5 N HCl (10 mL) and THF (10 mL) at 0° C. The mixture was extracted with ethyl acetate (2×50 mL), and the organic layer washed with water and brine and dried over anhydrous sodium sulfate to afford product 1502 (1.30 g, 92.8%). Mass: 240 [M+H].

### Synthesis of Compound 3

To a stirred solution of compound 103 (38 mg, 0.09 mmol) in DCM (5 mL) was added Dess-Martin reagent (87 mg, 0.46 mmol) at room temperature. The reaction mixture was stirred at room temperature for 3 hrs. The progress of the reaction mixture was monitored by TLC. After completion of the reaction, the reaction mixture was quenched with a saturated solution of sodium thiosulphate and saturated sodium bicarbonate and extracted with DCM (2×50 mL). The organic layer was washed with brine and dried over anhydrous sodium sulfate to afford crude product which was purified by column chromatography to obtain compound 3 with HPLC purity of 92.4%. Mass: 390 [M+]; ¹H NMR (200 MHz, CDCl₃): δ 1.5-2.2 (m, 15H), 5.1 (s, 2H), 6.8 (bs, 1H), 7.2-8.0 (m, 9H).

### Example 15

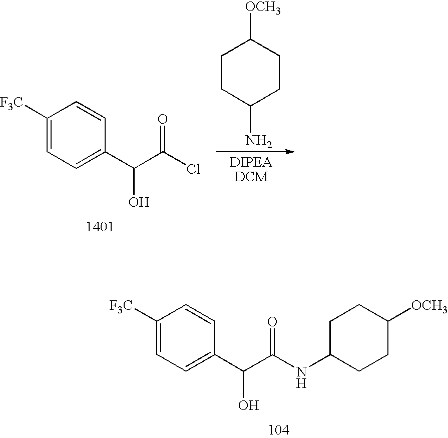

### Synthesis of Compound 104

To a stirred solution of compound 1401 (238 mg, 0.99 mmol) in DCM (5 mL) was added DIPEA (1.0 mL, 5.94 mmol) and p-methoxycyclohexylamine (140 mg, 1.08 mmol) at 0° C. The reaction mixture stirred at room temperature overnight. The progress of the reaction mixture was monitored by TLC. After completion of the reaction, the reaction mixture was concentrated under vacuum to remove DCM completely. The residue was dissolved in water and extracted with ethyl acetate (2×50 mL), and the organic layer was washed with brine and dried over anhydrous sodium sulfate to afford coupled product which was purified with preparative HPLC to obtain compound 104 with HPLC purity of 97.6%. Mass: 332 [M+H]. ¹H NMR (200 MHz, CDCl₃): δ 1.3 (m, 4H); 2.0 (m, 4H); 3.1 (m, 1H); 3.3 (s, 3H,); 3.78 (m, 1H); 5.1 (d, 1H); 6.0 (bs, 1H); 7.58 (d, 2H); 7.62 (d, 2H).

### Example 16

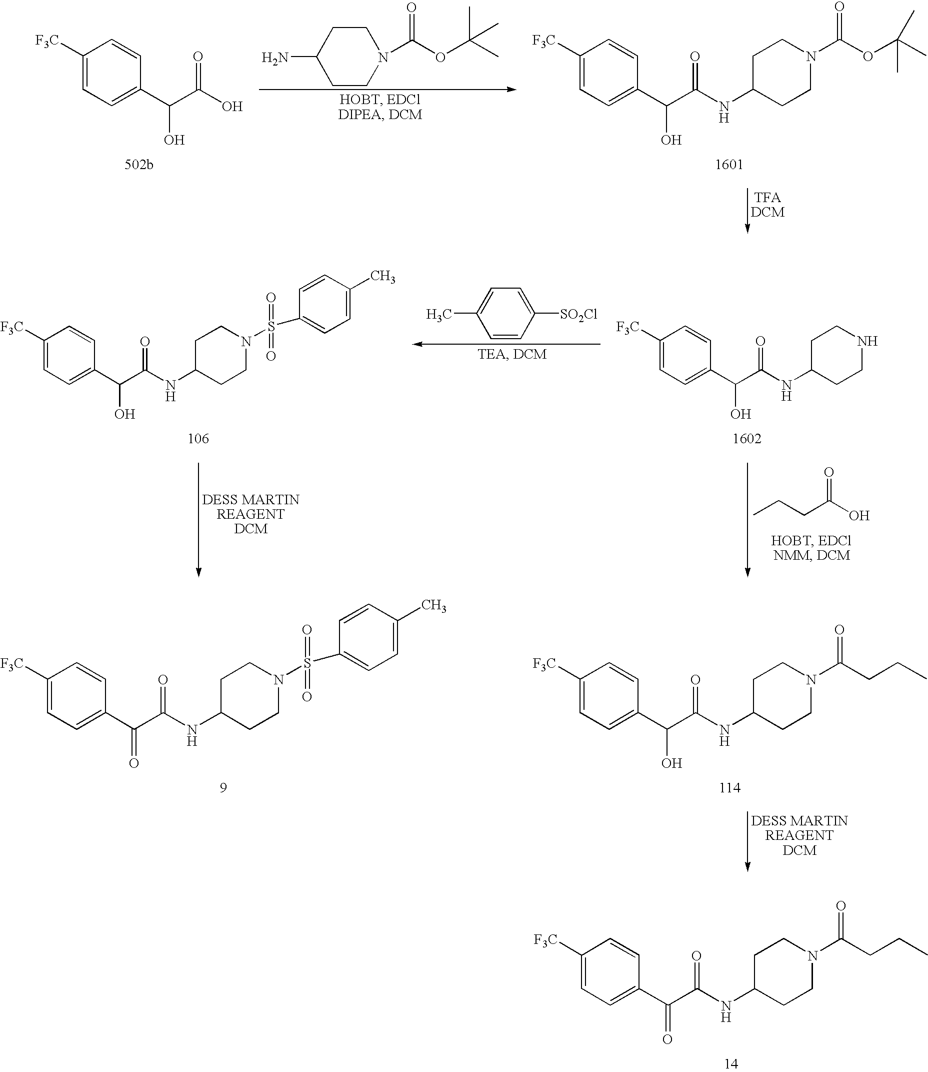

### Synthesis of Compound 1601

To a stirred solution of mandelic acid 502b (1.00 g, 5.00 mmol) in DCM (25 mL) was added 4-amino-piperidine-1-carboxylic acid tert-butyl ester (1.00 gm, 4.50 mmol), HOBT (680 mg, 5.00 mmol), EDCI (1.43 gm, 7.50 mmol) and DIPEA (4.5 mL, 25 mmol) at 0° C. The reaction mixture was stirred at room temperature overnight. The progress of the reaction mixture was monitored by TLC. After completion of the reaction, the reaction mixture was concentrated under vacuum to remove DCM. The residue was dissolved in water and extracted with ethyl acetate (2×50 mL), and the organic layer was washed with brine and dried over anhydrous sodium sulfate to get compound 1601 (1.7 g, 94%). Mass: 402 [M+H]; ¹H NMR (200 MHz, CDCl₃): δ 1.58 (s, 9H), 2.0 (m, 2H), 3.6 (m, 1H), 4.18 (m, 3H), 5.22 (d, 1H), 6.39 (d, 1H), 7.75 (d, 2H), 7.8 (d, 2H).

### Synthesis of Compound 1602

To a stirred solution of compound 1601 (324 mg, 0.806 mmol) in DCM (10 mL) was added trifluoroacetic acid (0.30 mL, 1.61 mmol) at 0° C. The reaction mixture was stirred at room temperature for 4 hrs. The progress of the reaction mixture was monitored by TLC. After completion of the reaction, the reaction mixture was quenched with saturated sodium bicarbonate solution and extracted with DCM (2×50 mL). The organic layer washed with brine solution and dried over anhydrous sodium sulfate to afford compound 1602 (200 mg, 82%).

### Synthesis of Compound 108

To a stirred solution of compound 1602 (140 mg, 0.46 mmol) in DCM (10 mL) was added TEA (0.2 mL) and p-toluenesulphonyl chloride (88 mg, 0.46 mmol) at 0° C. The reaction mixture was stirred at room temperature overnight. The progress of the reaction mixture was monitored by TLC. After completion of the reaction, the reaction mixture was concentrated under vacuum to remove DCM, and the residue was dissolved in water and extracted with ethyl acetate (2×50 mL). The organic layer was washed with brine and dried over anhydrous sodium sulfate to afford the coupled product which was purified by column chromatography to obtain compound 108 with HPLC purity of 93.7%. Mass: 457 [M+H]. ¹H NMR (500 MHz, CDCl₃): δ 1.58 (m, 2H), 1.89 (m, 2H), 2.38 (m, 2H), 2.42 (s, 3H), 3.38 (s, 1H), 3.78 (m, 3H), 5.1 (d, 1H), 6.38 (d, 1H), 7.38 (d, 8H), 7.58 (d, 2H), 7.62 (d, 4H).

### Synthesis of Compound 9

To a stirred solution of compound 108 (60 mg, 0.13 mmol) in DCM (7 mL) was added Dess-Martin reagent (117 mg, 0.270 mmol) at room temperature. The reaction mixture was stirred at room temperature for 3 hrs. The progress of the reaction was monitored by TLC. After completion of the reaction, the reaction mixture was quenched with saturated a solution of sodium thiosulphate and saturated sodium bicarbonate and extracted with DCM (2×50 mL). The organic layer was washed with brine and dried over anhydrous sodium sulfate to afford coupled product which was purified by column chromatography to give compound 9 (20 mg, 34%) with HPLC purity of 90.9%. Mass: 455 [M+H]; ¹H NMR (500 MHz, CDCl₃): δ 1.65 (m, 2H), 2.1 (m, 2H), 2.41 (s, 3H), 2.5 (m, 2H), 3.6 (m, 1H), 3.8 (m, 3H), 7.0 (bs, 1H), 7.4 (d, 2H), 7.62 (d, 2H), 7.78 (d, 2H), 8.41 (d, 2H).

### Synthesis of Compound 114

To a stirred solution of compound 1602 (200 mg, 0.660 mmol) in DCM (10 mL) was added butyric acid (53 mg, 0.61 mmol), HOBT (90 mg, 0.66 mmol), EDCI (190 mg, 0.990 mmol), and N-methylmorpholin (0.35 mL, 3.32 mmol) at 0° C. The reaction mixture was stirred at room temperature overnight. The progress of the reaction was monitored by TLC. After completion of the reaction, the reaction mixture was concentrated under vacuum to remove DCM. The residue was dissolved in water and extracted with ethyl acetate (2×50 mL), and the organic layer was washed with brine and dried over anhydrous sodium sulfate to afford coupled product which was purified by column chromatography to give compound 114 (120 mg, 95.7%) with HPLC purity of 95.7%. Mass: 373 [M+H]. ¹H NMR (500 MHz, CDCl₃): δ 1.0 (t, 3H), 1.3 (m, 2H), 1.98 (m, 2H), 2.3 (m, 2H), 2.7 (m, 1H), 3.1 (m, 1H), 3.45 (bs, 1H), 3.8 (m, 1H), 4.0 (m, 1H), 4.58 (m, 1H), 5.1 (d, 1H), 6.3 (m, 1H), 7.6 (d, 2H), 7.7 (d, 2H).

### Synthesis of Compound 14

To a stirred solution of compound 114 (110 mg, 0.29 mmol) in DCM (10 mL) was added Dess-Martin reagent (263 mg, 0.62 mmol) at room temperature. The reaction mixture was stirred at room temperature for 3 hrs. The progress of the reaction was monitored by TLC. After completion of the reaction, the reaction mixture was quenched with a saturated solution of sodium thiosulphate and saturated sodium bicarbonate and extracted with DCM (2×50 mL). The organic layer was washed with brine and dried over anhydrous sodium sulfate to afford coupled product which was purified by column chromatography to give compound 14 (15 mg, 14%) with HPLC purity of 91.8%. Mass: 371 [M+H]. ¹H NMR (500 MHz, CDCl₃): δ 1.0 (m, 3H), 1.4-1.8 (m, 6H), 2.1 (m, 2H), 2.38 (m, 2H), 2.8 (m, 1H), 3.2 (m, 1H), 3.9 (m, 1H), 4.1 (m, 1H), 4.6 (m, 1H), 7.08 (bs, 1H), 7.78 (d, 2H), 8.42 (d, 2H).

### Example 17

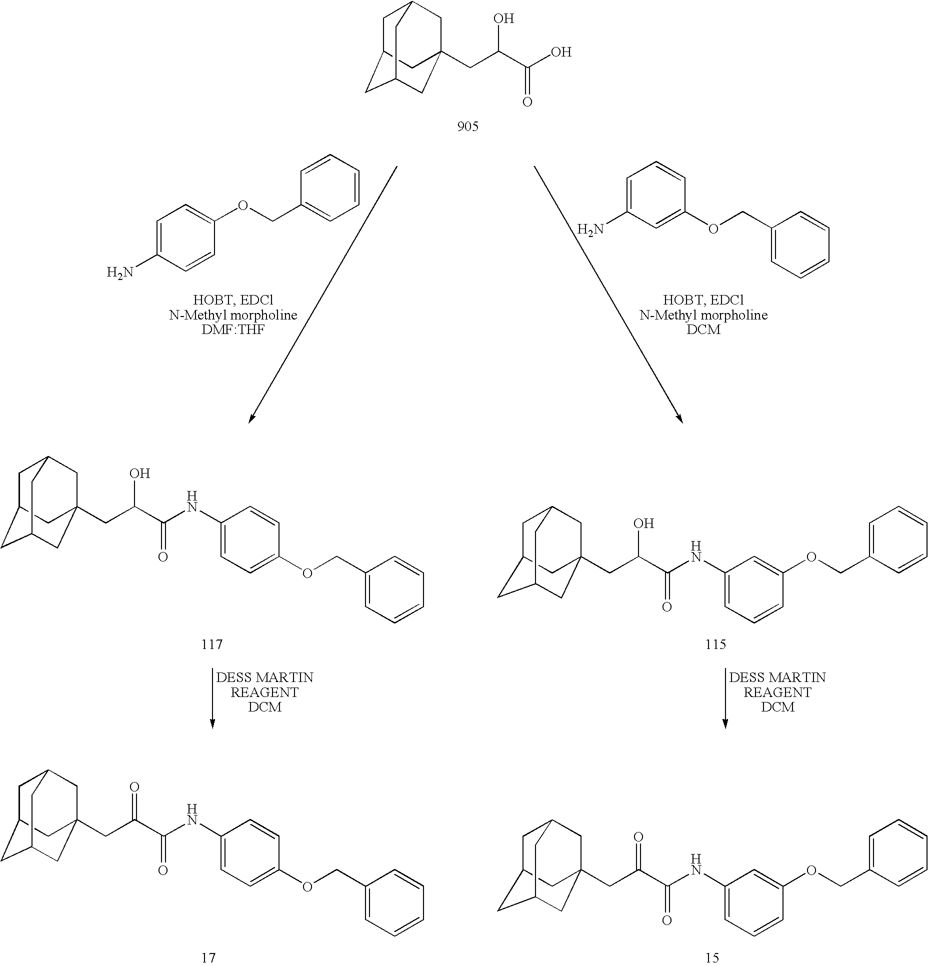

### Synthesis of Compound 115

To a stirred solution of compound 905 (120 mg, 0.54 mmol) in DCM (10 mL) was added 3-benzyloxy-phenylamine (107 mg, 0.54 mmol), HOBT (73 mg, 0.54 mmol), EDCI (154 mg, 0.81 mmol), and N-methylmorpholine (0.30 mL, 2.70 mmol) at 0° C. The reaction mixture was stirred at room temperature overnight. The progress of the reaction was monitored by TLC. After completion of the reaction, the reaction mixture was concentrated under vacuum to remove DCM. The residue was dissolved in water and extracted with ethyl acetate (2×50 mL), and the organic layer was washed with brine and dried over anhydrous sodium sulfate to afford coupled product which was purified by column chromatography to give compound 115 (20 mg, 9.2%) with HPLC purity of 97.7%. Mass: 406 [M+H]. ¹H NMR (500 MHz, CDCl₃): δ 1.4-2.0 (m, 16H), 2.21 (d, 1H), 4.4 (d, 1H), 5.08 (s, 2H), 6.78 (m, 1H), 7.1 (m, 1H), 7.2-7.6 (m, 7H), 8.41 (bs, 1H).

### Synthesis of Compound 117

To a stirred solution of compound 905 (500 mg, 2.23 mmol) in dry THF (5 mL) and DMF (10 mL) was added 4-benzyloxy-phenylamine, DIPEA (0.38 mL, 2.23 mmol), HOBT (300 mg, 2.23 mmol), and EDCI (639 mg, 3.34 mmol) at 0° C. The reaction mixture was stirred at room temperature overnight. The progress of the reaction was monitored by TLC. After completion of the reaction, the reaction mixture was concentrated under vacuum to remove THF and DMF completely. The residue was dissolved in water and extracted with ethyl acetate (2×50 mL), and the organic layer was washed with brine and dried over anhydrous sodium sulfate to afford coupled product which was purified by column chromatography on silica gel eluting with 12% ethyl acetate in hexanes, to give compound 117 (140 mg, 15.4%). Mass: 406 [M+H]. ¹H NMR (500 MHz, CDCl₃): δ 1.4-2.0 (m, 16H), 2.21 (d, 1H), 4.4 (d, 1H), 5.08 (s, 2H), 6.98 (d, 2H), 7.2-7.6 (m, 7H), 8.39 (bs, 1H).

### Synthesis of Compound 17

To a stirred solution of compound 117 (140 mg, 0.340 mmol) in DCM (10 mL) was added Dess-Martin reagent (235 mg, 0.720 mmol) at room temperature. The reaction mixture was stirred at room temperature for 3 hrs. The progress of the reaction was monitored by TLC. After completion of the reaction, the reaction mixture was quenched with a saturated solution of sodium thiosulphate and saturated sodium bicarbonate and extracted with DCM (2×50 mL). The organic layer was washed with brine and dried over anhydrous sodium sulfate to afford coupled product which was purified by column chromatography on silica gel eluting with 10% ethyl acetate in hexanes to give compound 17 (40 mg, 29%) with HPLC purity of 99.4%. Mass: 404 [M+H]. ¹H NMR (500 MHz, CDCl₃): δ 1.4-2.0 (m, 15H), 2.8 (s, 2H), 5.08 (s, 2H), 6.98 (m, 2H), 7.1 (m, 1H), 7.2-7.6 (m, 6H), 8.7 (bs, 1H).

### Synthesis of Compound 15

Compound 15 was prepared in a similar manner as compound 17 by oxidation of compound 115 with the Dess-Martin reagent.

### Example 18

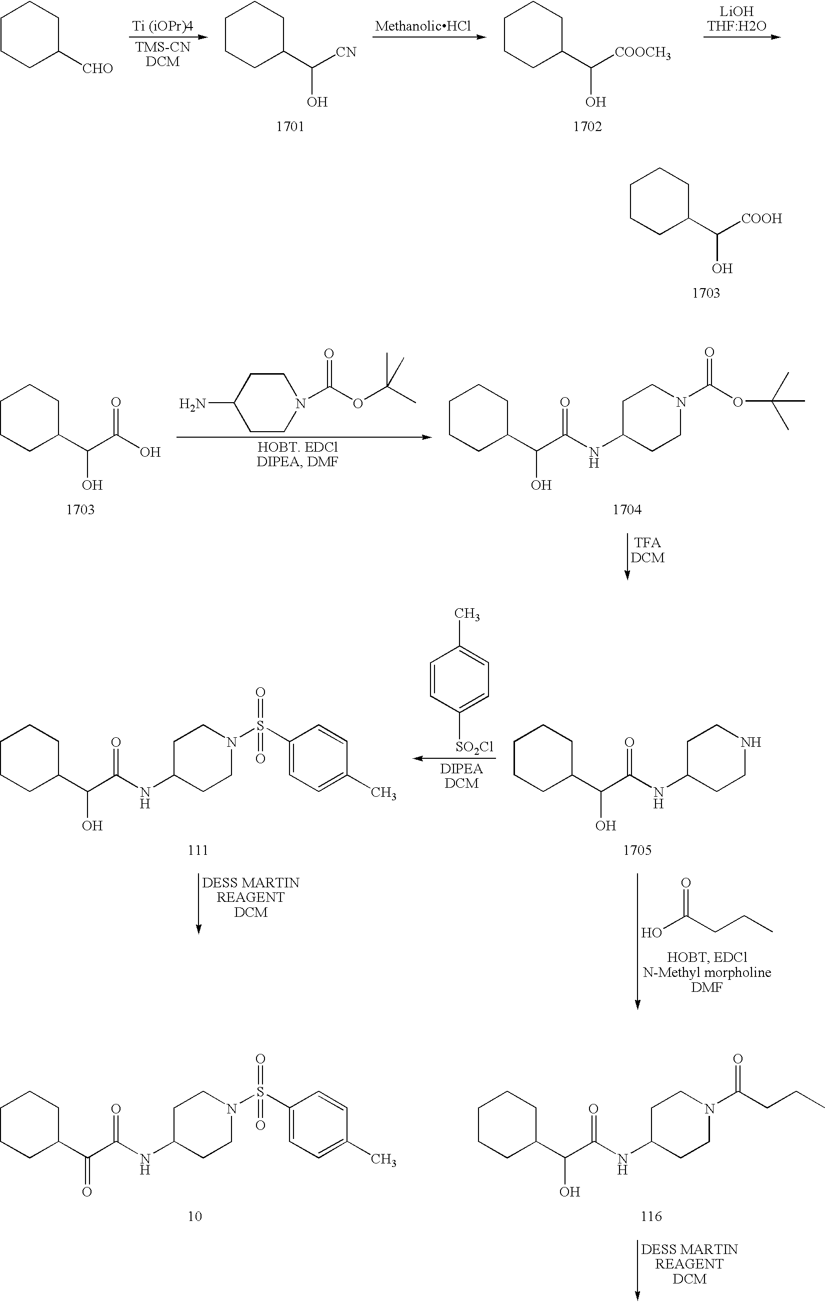

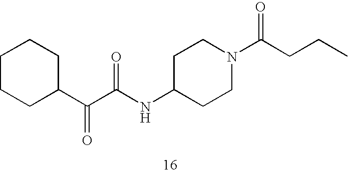

### Synthesis of Compound 1701

To a stirred solution of cyclohexylcarbaldehyde (4.30 mL, 35.7 mmol) in DCM (30 mL) was added Ti(iOPr)₄ (4.21 mL, 14.3 mmol) at 0° C. The reaction mixture was maintained at room temperature, TMSCN (16.7 mL, 125 mmol) was added, and the reaction mixture was stirred at room temperature for 4 hrs. The progress of the reaction mixture was monitored by TLC. After completion, the reaction mixture was quenched with 1.5 N HCl (10 mL) and THF (10 mL) at 0° C., and extracted with ethyl acetate (2×50 mL). The organic layer was washed with water and brine and dried over anhydrous sodium sulfate to afford product 1701 (3.2 g, 65%). Mass: 140 [M+H]; ¹H NMR (200 MHz, CDCl₃): δ 1.0-1.4 (m, 5H), 1.5-2.05 (m, 6H), 4.3 (d, 1H).

### Synthesis of Compound 1702

To compound 1701 (3.2 gm, 23 mmol) was added methanolic HCl (30 mL), and the reaction mixture heated to 70° C.-80° C. for 4 hrs. The progress of the reaction was monitored by TLC. After completion, the reaction mixture was concentrated under vacuum to remove the solvent, and the residue was extracted with ethyl acetate (2×50 mL). The organic layer was washed with water and brine and dried over anhydrous sodium sulfate to afford product 1702 (2.7 g, 68%). Mass: 173 [M+H]; ¹H NMR (200 MHz, CDCl₃): δ 1.0-1.4 (m, 6H); 1.5-2.05 (m, 5H); 3.8 (s, 3H); 4.1 (d, 1H).

### Synthesis of Compound 1703

To a stirred solution of compound 1702 (2.7 g, 15.7 mmol) in THF (10 mL) and H₂O (10 mL) was added lithium hydroxide (1.97 gm, 47.1 mmol) at 0° C. The reaction mixture was stirred at room temperature for 4 hrs. The progress of the reaction was monitored by TLC. After completion, the reaction mixture was concentrated under vacuum to remove THF, and the residue obtained was adjusted to pH 2 at 0° C. and extracted with ethyl acetate (2×50 mL). The organic layer was washed with water, brine and dried over anhydrous sodium sulfate to get the product 1703 (1.8 g, 73%). ¹HNMR (200 MHz, DMSO-D6): δ 1.0-1.3 (m, 5H), 1.4-1.8 (m, 6H), 3.8 (d, 1H).

### Synthesis of Compound 1704

To a stirred solution of compound 1703 (1.70 gm, 10.7 mmol) in DMF (10 mL) was added 4-amino-piperidine-1-carboxylic acid tert-butyl ester (2.10 g, 10.8 mmol), HOBT (731 mg, 5.37 mmol), EDCI (4.10 gm, 21.5 mmol) and DIPEA (5.5 mL, 32.3 mmol) at 0° C. The reaction was mixture stirred at room temperature overnight. The progress of the reaction was monitored by TLC. After completion of the reaction, the reaction mixture was concentrated under vacuum to remove DMF completely. The residue was dissolved in water and extracted with ethyl acetate (2×50 mL), and the organic layer was washed with brine and dried over anhydrous sodium sulfate to afford the coupled product 1704 (1.0 g, 28%). Mass: 340 [M+H]; ¹H NMR (200 MHz, CDCl₃): δ 1.99 (t, 2H), 2.5 (m, 6H), 3.78 (m, 4H), 4.0 (t, 2H), 5.3 (d, 1H), 6.7 (d, 1H), 7.0 (d, 1H), 7.2 (d, 1H), 7.39 (m, 2H), 7.65 (s, 2H), 8.33 (s, 1H).

### Synthesis of Compound 1705

To a stirred solution of compound 1704 (300 mg, 0.88 mmol) in DCM (10 mL) was added trifluoroacetic acid (0.5 mL, 4.4 mmol) at 0° C. The reaction mixture was stirred at room temperature for 4 hrs. The progress of the reaction was monitored by TLC. After completion of the reaction, the reaction mixture was quenched with a saturated solution of sodium bicarbonate and extracted with DCM (2×50 mL). The organic layer was washed with brine and dried over anhydrous sodium sulfate to afford product 1705.

### Synthesis of Compound 111

To a stirred solution of compound 1705 (300 mg, 1.25 mmol) in DCM (10 mL) was added DIPEA (1.00 mL, 4.41 mmol) and p-toluene sulfonylchloride (239 mg, 1.25 mmol) at 0° C. The reaction mixture was stirred at room temperature overnight. The progress of the reaction was monitored by TLC. After completion of the reaction, the reaction mixture was concentrated under vacuum to remove DCM. The residue was dissolved in water and extracted with ethyl acetate (2×50 mL), and the organic layer was washed with brine and dried over anhydrous sodium sulfate to afford coupled product 111 (153 mg, 44%) with HPLC purity of 99.5%. Mass: 391 [M+H]; ¹H NMR (200 MHz, CDCl₃): δ 1.1-1.4 (m, 6H), 1.42 (m, 1H), 1.6-1.8 (m, 5H), 2.0 (m, 2H), 2.2 (d, 1H), 3.78 (m, 3H), 3.98 (d, 1H), 6.4 (d, 1H), 7.38 (d, 2H), 7.65 (d, 2H).

### Synthesis of Compound 10

To a stirred solution of compound III (100 mg, 0.25 mmol) in DCM (10 mL) was added Dess-Martin reagent (226 mg, 0.530 mmol) at room temperature. The reaction mixture was stirred at room temperature for 3 hrs. The progress of the reaction mixture was monitored by TLC. After completion of the reaction, the reaction mixture was quenched with a saturated solution of sodium thiosulphate and saturated sodium bicarbonate and extracted with DCM (2×50 mL). The organic layer was washed with brine and dried over anhydrous sodium sulfate to afford coupled product 10 (38 mg, 38%) with HPLC purity of 97.6%. Mass: 393 [M+H]; ¹H NMR (500 MHz, CDCl3): δ 1.1-1.4 (m, 7H), 1.6 (m, 2H), 1.7 (m, 1H), 1.8 (m, 4H), 2.0 (m, 2H), 2.45 (m, 5H), 3.48 (m, 1H), 3.75 (m, 3H), 6.8 (d, 1H), 7.4 (d, 2H), 7.65 (d, 2H).

### Synthesis of Compound 116

To a stirred solution of compound 1705 (353 mg, 1.47 mmol) in DMF (10 mL) was added butyric acid (0.10 mL, 1.17 mmol), HOBT (200 mg, 1.47 mmol), EDCI (561 mg, 2.94 mmol), and N-methylmorpholin (0.50 mL, 4.41 mmol) at 0° C. The reaction mixture was stirred at room temperature overnight. The progress of the reaction was monitored by TLC. After completion of the reaction, the reaction mixture was concentrated under vacuum to remove DMF completely. The residue was dissolved in water and extracted with ethyl acetate (2×50 mL), and the organic layer washed with brine and dried over anhydrous sodium sulfate to afford coupled product 116 (25 mg, 7.1%) with HPLC purity of 98.6%. Mass: 311 [M+H]; ¹H NMR (500 MHz, CDCl₃): δ 0.98 (t, 3H), 1.1-1.4 (m, 9H), 1.45 (m, 1H), 1.6-2.0 (m, 7H), 2.3 (m, 3H), 2.77 (t, 1H), 3.18 (t, 1H), 3.81 (d, 1H), 4.1 (m, 1H), 4.38 (m, 1H), 6.4 (m, 1H).

### Synthesis of Compound 16

To the stirred solution of compound 116 (200 mg, 0.64 mmol) in DCM (10 mL) was added Dess-Martin reagent (524 mg, 1.23 mmol) at room temperature. The reaction mixture was stirred at room temperature for 3 hrs. The progress of the reaction was monitored by TLC. After completion of the reaction, the reaction mixture was quenched with a a saturated solution of sodium thiosulphate and saturated sodium bicarbonate and extracted with DCM (2×50 mL), and the organic layer was washed with brine and dried over anhydrous sodium sulfate to afford coupled product 16 (23 mg, 12%) with HPLC purity of 97.7%. Mass: 309 [M+H]; ¹H NMR (500 MHz, CDCl₃): δ 0.98 (t, 3H), 1.1-1.4 (m, 8H), 1.6-2.0 (m, 10H), 2.3 (m, 3H), 2.77 (t, 1H), 3.18 (t, 1H), 3.4 (t, 1H), 3.85 (d, 1H), 4.0 (m, 1H), 4.38 (m, 1H), 6.85 (m, 1H).

### Example 19

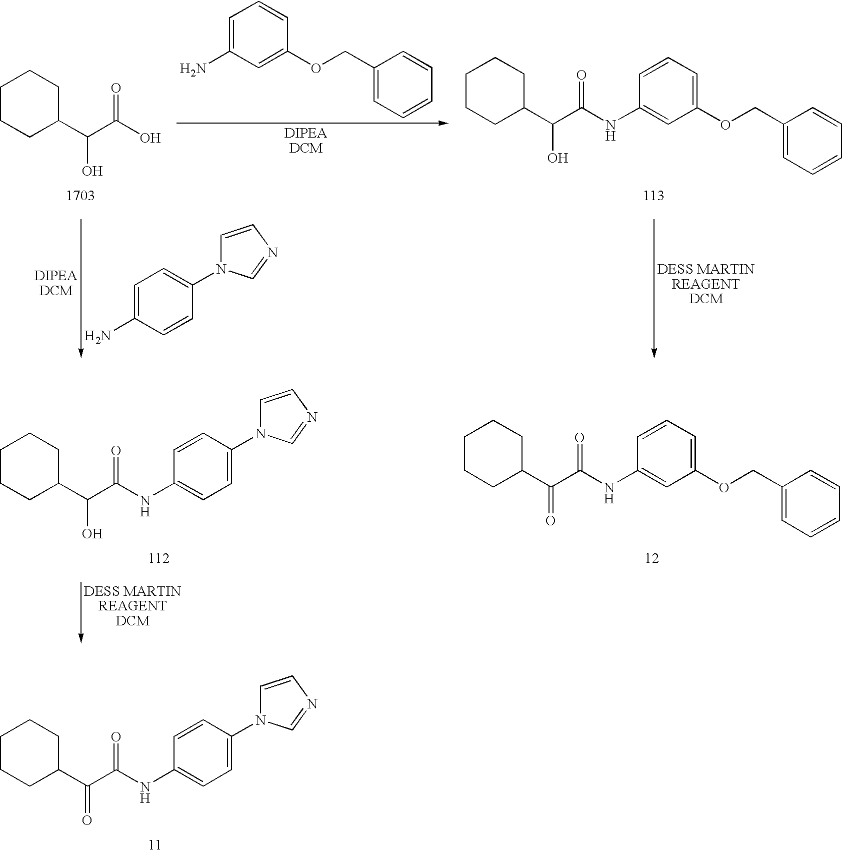

### Synthesis of Compound 113

To a stirred solution of compound 1703 (200 mg, 1.26 mmol) in DCM (10 mL) was added N-methylmorpholine (0.41 mL, 3.79 mmol) and 3-benzyloxy-phenylamine (251 mg, 1.26 mmol) at 0° C. The reaction mixture was stirred at room temperature overnight. The progress of the reaction was monitored by TLC. After completion of the reaction, the reaction mixture was concentrated under vacuum to remove DCM. The residue was dissolved in water and extracted with ethyl acetate (2×50 mL), and the organic layer was washed with brine and dried over anhydrous sodium sulfate to afford coupled product 113 (120 mg, 28%) with HPLC purity of 91.4%. Mass: 340 [M+H]; ¹H NMR (500 MHz, CDCl₃): δ 1.35 (m, 5H), 1.6-1.85 (m, 6H), 4.1 (d, 1H), 5.1 (s, 2H), 6.78 (m, 1H), 7.08 (m, 1H), 7.2-7.6 (m, 7H), 8.38 (s, 1H).

### Synthesis of Compound 12

To a stirred solution of compound 113 (175 mg, 0.510 mmol) in DCM (10 mL) was added Dess-Martin reagent (459 mg, 1.08 mmol) at room temperature. The reaction mixture was stirred at room temperature for 3 hrs. The progress of the reaction was monitored by TLC. After completion of the reaction, the reaction mixture was quenched with an equal mixture of saturated sodium thiosulphate (10 mL) and saturated sodium bicarbonate solution (10 mL) and extracted with DCM (2×50 mL). The organic layer was washed with brine and dried over anhydrous sodium sulfate to afford coupled product 12 (50 mg, 29%) with HPLC purity of 99.6%. Mass: 338 [M+H]; ¹H NMR (500 MHz, CDCl₃): δ 1.35 (m, 5H), 1.6-1.85 (m, 6H), 3.5 (m, 1H), 5.1 (s, 2H), 6.8 (m, 1H), 7.1 (m, 1H), 7.2-7.6 (m, 7H), 8.78 (s, 1H).

### Synthesis of Compound 112

To a stirred solution of compound 1703 (175 mg, 1.10 mmol) in DCM (10 mL) was added N-methylmorpholine (0.36 mL, 3.32 mmol) and 4-imidazol-1-yl-phenylamine compound (141 mg, 0.88 mmol) at 0° C. The reaction mixture was stirred at room temperature for 4 hrs. The progress of the reaction was monitored by TLC. After completion of the reaction, the reaction mixture was concentrated under vacuum to remove DCM. The residue was dissolved in water, extracted with ethyl acetate (2×50 mL), and the organic layer was washed with brine and dried over anhydrous sodium sulfate to afford coupled product 112 (60 mg, 18%) with HPLC purity of 99.5%. Mass: 300 [M+H]; ¹H NMR (500 MHz, CDCl₃): δ 1.0-1.3 (m, b, 5H), 1.5-1.8 (m, 6H), 3.8 (d, 1H), 7.1 (s, 1H), 7.58 (d, 2H), 7.7 (s, 1H), 7.84 (d, 2H), 8.2 (s, 1H), 9.8 (s, 1H).

### Synthesis of Compound 11

To a stirred solution of compound 112 (165 mg, 0.55 mmol) in DCM (10 mL) was added Dess-Martin reagent (491 mg, 1.15 mmol) at room temperature. The reaction mixture was stirred at room temperature for 3 hrs. The progress of the reaction was monitored by TLC. After completion of the reaction, the reaction mixture was quenched with a saturated solution of sodium thiosulphate (10 mL) and saturated sodium bicarbonate solution (10 mL) and extracted with DCM (2×50 mL). The organic layer was washed with brine and dried over anhydrous sodium sulfate to afford compound 11 (70 mg, 43%) with HPLC purity of 98.5%. Mass: 298 [M+H]; ¹H NMR (500 MHz, CDCl₃): δ 1.0-1.3 (m, 5H), 1.5-1.8 (m, 5H), 3.5 (m, 1H), 7.2 (s, 1H), 7.42 (d, 2H), 7.8 (d, 2H), 7.82 (s, 1H), 8.9 (s, 1H).

### Example 20

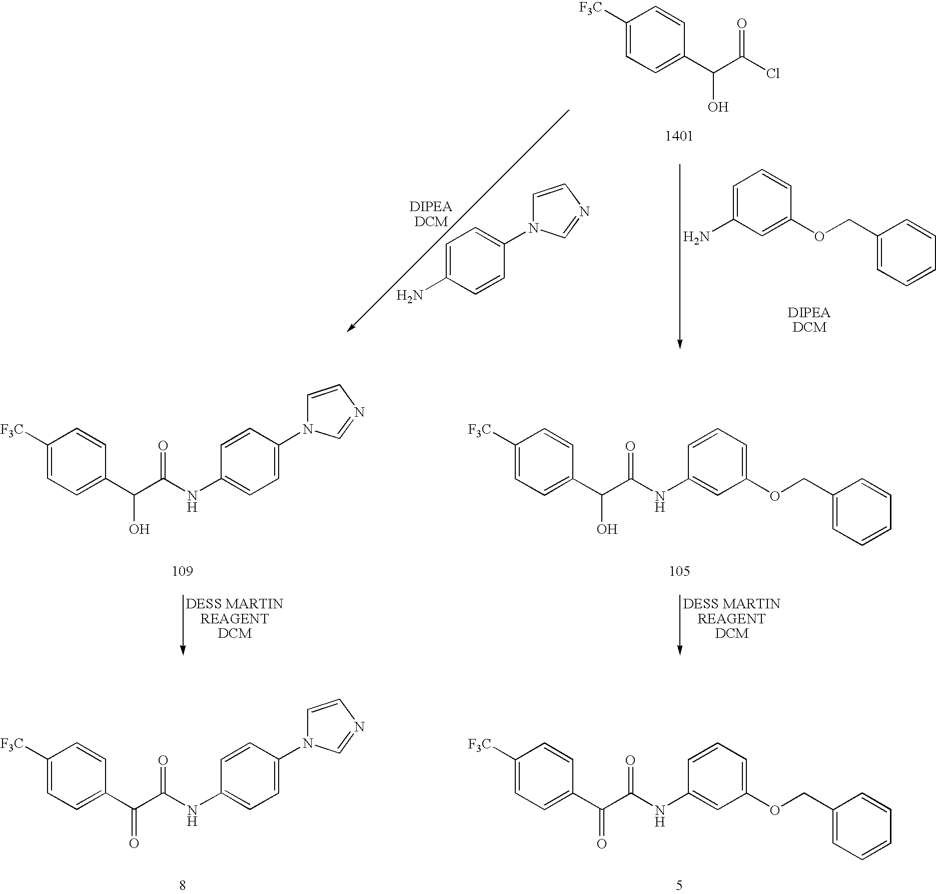

### Synthesis of Compound 105

To a stirred solution of compound 1401 (433 mg, 1.81 mmol) in DCM (15 mL) was added DIPEA (1.17 mL, 9.09 mmol) and 3-benzyloxyphenylamine (361 mg, 1.81 mmol) at 0° C. The reaction mixture was stirred at room temperature overnight. The progress of the reaction was monitored by TLC. After completion of the reaction, the reaction mixture was concentrated under vacuum to remove DCM. The residue was dissolved in water and extracted with ethyl acetate (2×50 mL), and the organic layer was washed with brine and dried over anhydrous sodium sulfate to afford coupled product 105 (100 mg, 13%) with HPLC purity of 96.0%. Mass: 402 [M+H]. ¹H NMR (200 MHz, CDCl₃): δ 3.4 (bs, 1H), 5.05 (s, 2H), 5.3 (s, 1H), 6.78 (m, 1H), 7.0 (m, 1H), 7.2-7.7 (m, 11H), 8.21 (bs, 1H).

### Synthesis of Compound 5

To a stirred solution of compound 105 (85 mg, 0.22 mmol) in DCM (5 mL) was added Dess-Martin reagent (89 mg, 0.44 mmol) at room temperature. The reaction mixture was stirred at room temperature for 3 hrs. The progress of the reaction was monitored by TLC. After completion of the reaction, the reaction mixture was quenched with a saturated solution of sodium thiosulphate (10 mL) and saturated sodium bicarbonate solution (10 mL) and extracted with DCM (2×50 mL). The organic layer was washed with brine and dried over anhydrous sodium sulfate to afford coupled product 5 (30 mg, 35%) with HPLC purity of 99.1%. Mass: 400 [M+H]; ¹H NMR (200 MHz, CDCl₃): δ 5.05 (s, 2H), 6.82 (m, 1H), 7.2-7.8 (m, 10H), 8.58 (m, 2H), 8.88 (bs, 1H).

### Synthesis of Compound 109

To a stirred solution of compound 1401 (542 mg, 2.27 mmol) in DCM (10 mL) was added DIPEA (0.95 mL, 6.81 mmol) and 4-imidazol-1-yl-phenylamine (325 mg, 2.05 mmol) at 0° C. The reaction mixture was stirred at room temperature for 4 hrs. The progress of the reaction was monitored by TLC. After completion of the reaction, the reaction mixture was concentrated under vacuum to remove DCM. The residue was dissolved in water and extracted with ethyl acetate (2×50 mL), and the organic layer was washed with brine solution and dried over anhydrous sodium sulfate to afford coupled product 109 (100 mg, 12.2%) with HPLC purity of 97.9%. Mass: 362 [M+H]; ¹H NMR (200 MHz, CDCl₃): δ 5.21 (d, 1H), 6.78 (d, 1H), 7.06 (s, 1H), 7.5 (d, 2H), 7.6 (s, 1H), 7.82 (d, 2H), 8.2 (s, 1H), 10.2 (s, 1H).

### Synthesis of Compound 8

To a stirred solution of compound 109 (90 mg, 0.22 mmol) in DCM (10 mL) was added Dess-Martin reagent (317 mg, 0.74 mmol) at room temperature. The reaction mixture was stirred at room temperature for 3 hrs. The progress of the reaction was monitored by TLC. After completion of the reaction, the reaction mixture was quenched with a saturated solution of sodium thiosulphate (10 mL) and saturated sodium bicarbonate (10 mL) and extracted with DCM (2×50 mL). The organic layer washed with brine and dried over anhydrous sodium sulfate to afford coupled product 8 (45 mg, 50%) with HPLC purity of 99.7%. Mass: 360 [M+H]; ¹H NMR (200 MHz, CDCl₃): δ 7.25 (m, 3H), 7.45 (d, 2H), 7.8 (d, 2H), 7.9 (d, 2H), 8.6 (d, 2H), 9.2 (s, 1H).

### Example 21

Compound 129 was prepared in a fashion similar to that of Compound 125 by coupling the corresponding acid to the corresponding amine. HPLC purity: 96.1%. Mass: 448 [M+1]; ¹H NMR (200 MHz, CDCl₃): δ 7.45 (m, 5H), 7.18 (d, J=3.5 Hz, 2H), 6.80 (d, J=3.5 Hz, 2H), 6.35 (s, 1H), 5.10 (s, 2H), 4.02 (m, 1H), 3.50 (m, 2H), 3.81 (m, 2H), 1.89 (s, 3H), 1.60-1.80 (m, 6H), 1.40 (s, 6H), 1.08 (m, 2H).

The following compounds were prepared according to procedures similar to those described above.

| | | Mass | Purity |
|---|---|---|---|
| Compound | Structure | [M + 1] | HPLC |
| 20 | 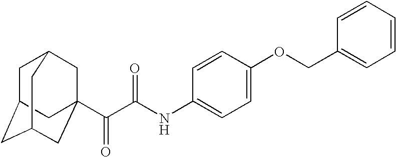 | 390 | 93.3 |
| 21 | 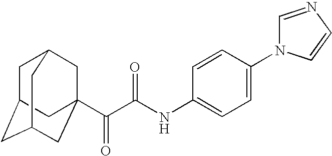 | 350 | 85.8 |
| 22 | 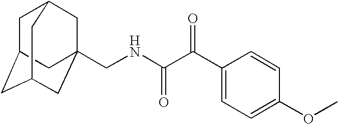 | 328 | 97.8 |
| 23 | 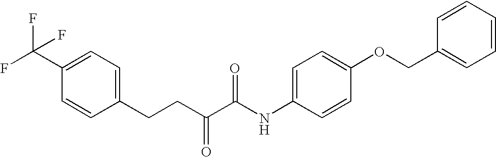 | 428 | 99.7 |
| 24 | 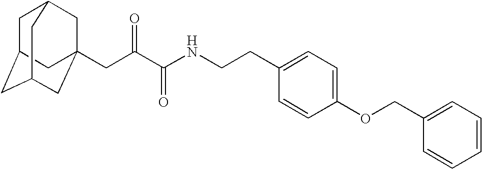 | 432 | 99.4 |
| 25 | 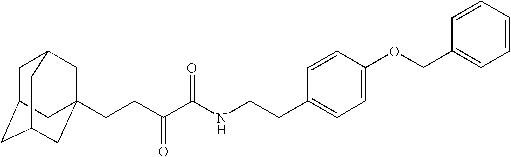 | 446 | 90.2 |
| 130 | 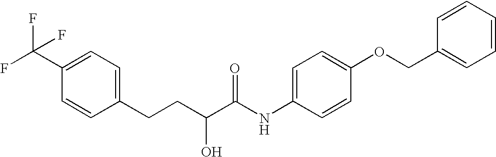 | 430 | 97.2 |
| 131 | 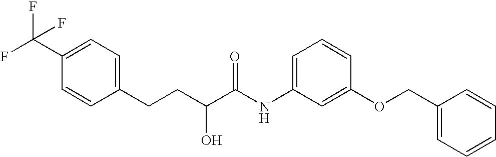 | 430 | 92.7 |
| 132 | 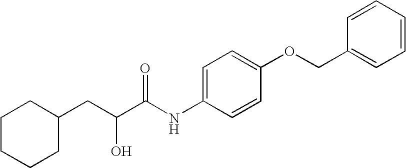 | 354 | 92.5 |
| 133 | 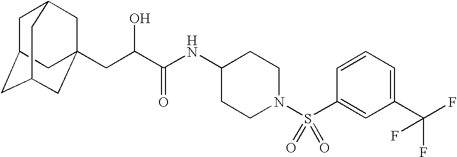 | 515 | 92.16 |
| 134 | 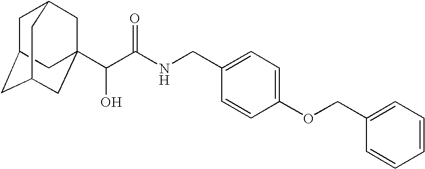 | 406 | 94.6 |
| 135 | 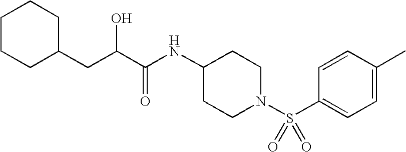 | 409 | 95.7 |
| 136 | 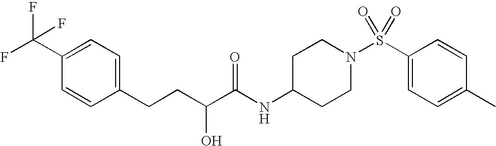 | 485 | 94.9 |
| 137 | 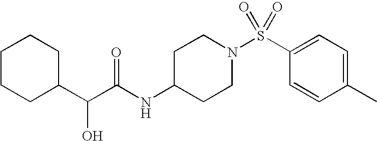 | 395 | 99.2 |
| 138 | 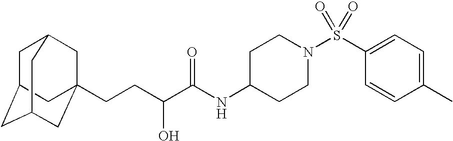 | 475 | 96.4 |
| 139 | 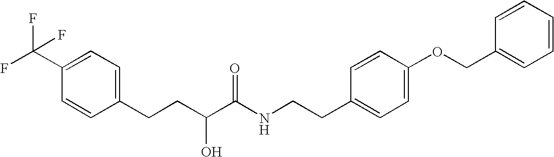 | 458 | 98.9 |
| 140 | 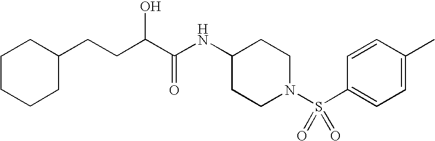 | 423 | 92.3 |
| 141 | 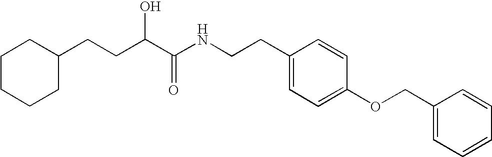 | 396 | 99.2 |
| 142 | 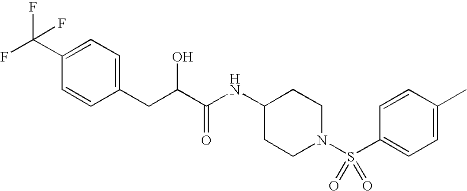 | 471 | 96.8 |
| 143 | 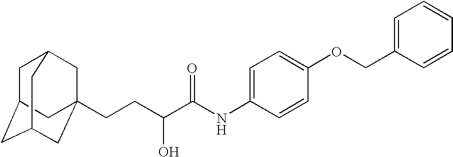 | 420 | 87.4 |
| 144 | 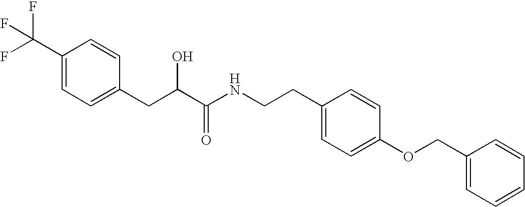 | 444 | 95.8 |
| 145 | 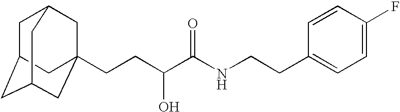 | 360 | 97.8 |
| 146 | 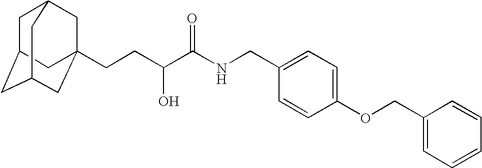 | 434 | 91.4 |
| 147 | 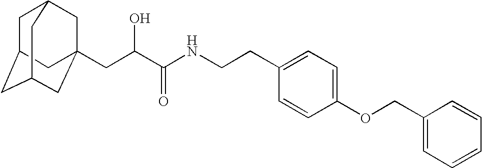 | 434 | 98.9 |
| 148 | 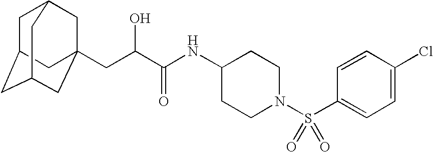 | 481 | 92.7 |
| 149 | 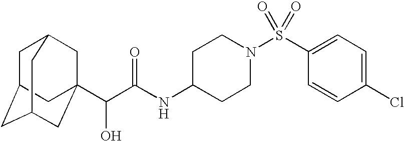 | 467 | 86.4 |
| 150 | 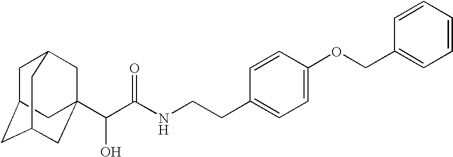 | 420 | 84.4 |
| 151 | 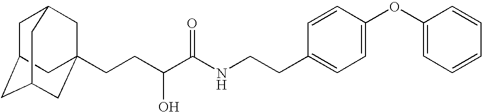 | 434 | 94.8 |
| 152 | 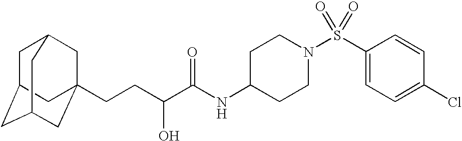 | 495 | 97.1 |
| 153 | 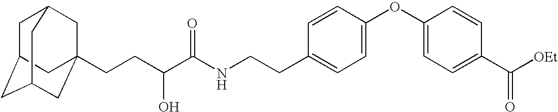 | 461 | 96.9 |
| 154 | 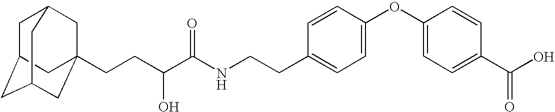 | 478 | 96.7 |
| 155 | 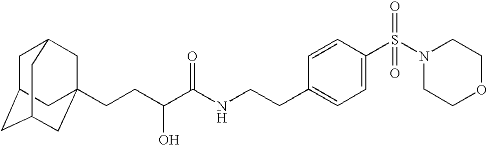 | 491 | 97.3 |
| 156 | 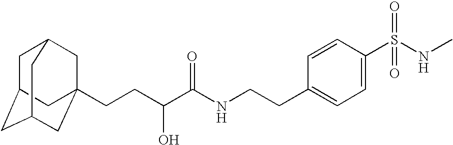 | 435 | 91.5 |
| 157 | 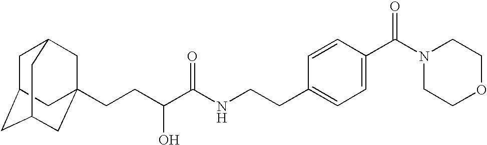 | 455 | 97.3 |
| 158 | 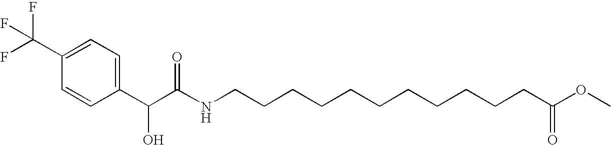 | 432 | 97.9 |
| 159 | 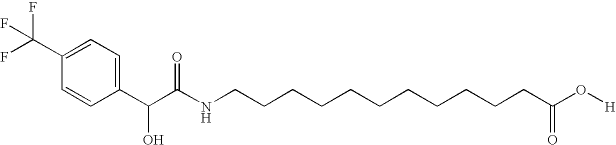 | 418 | 96.9 |
| 160 | 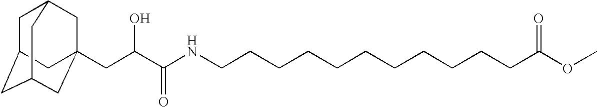 | 436 | 91.3 |
| 161 | 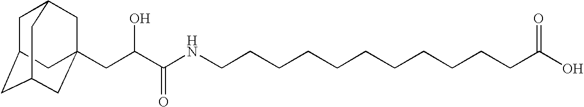 | 422 | 95.9 |
| 162 | 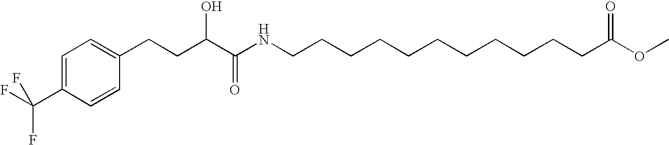 | 460 | 97.2 |
| 163 | 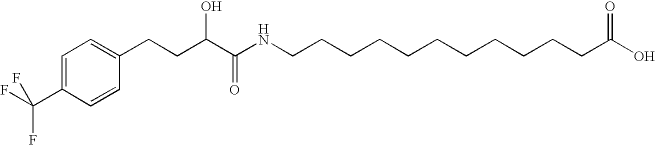 | 446 | 96.1 |
| 164 | 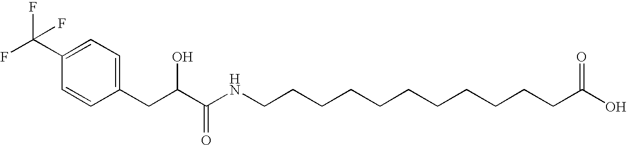 | 432 | 91.9 |
| 165 | 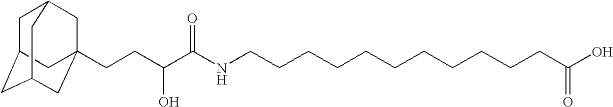 | 436 | 93.7 |

### BIOLOGICAL EXAMPLES

### Example 1

### Fluorescent Assay for Mouse and Human Soluble Epoxide Hydrolase

Recombinant mouse sEH (MsEH) and human sEH (HsEH) were produced in a baculovirus expression system as previously reported. Grant et al., J. Biol. Chem., 268:17628-17633 (1993); Beetham et al., Arch. Biochem. Biophys., 305:197-201 (1993). The expressed proteins were purified from cell lysate by affinity chromatography. Wixtrom et al., Anal. Biochem., 169:71-80 (1988). Protein concentration was quantified using the Pierce BCA assay using bovine serum albumin as the calibrating standard. The preparations were at least 97% pure as judged by SDS-PAGE and scanning densitometry. They contained no detectable esterase or glutathione transferase activity which can interfere with the assay. The assay was also evaluated with similar results in crude cell lysates or homogenate of tissues.

The IC50s for each inhibitor were according to the following procedure:

### Substrate:

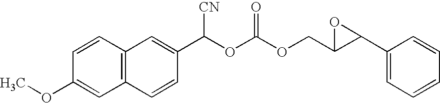

Cyano(2-methoxynaphthalen-6-yl)methyl (3-phenyloxiran-2-yl)methyl carbonate (CMNPC; Jones P. D. et. al.; Analytical Biochemistry 2005; 343: pp. 66-75)

### Solutions:

Bis/Tris HCl 25 mM pH 7.0 containing 0.1 mg/mL of BSA (buffer A)

CMNPC at 0.25 mM in DMSO.

Mother solution of enzyme in buffer A (Mouse sEH at 6 μg/mL and Human sEH at 5 μg/mL).

Inhibitor dissolved in DMSO at the appropriate concentration.

### Protocol:

In a black 96 well plate, fill all the wells with 150 μL of buffer A.

Add 2 μL of DMSO in well A2 and A3, and then add 2 μL of inhibitor solution in A1 and A4 through A12.

Add 150 μL of buffer A in row A, then mix several time and transfer 150 μL to row B. Repeat this operation up to row H. The 150 μL removed from row H is discarded.

Add 20 μL of buffer A in column 1 and 2, then add 20 μL of enzyme solution to column 3 to 12.

Incubate the plate for 5 minutes in the plate reader at 30° C.

During incubation prepare the working solution of substrate by mixing 3.68 mL of buffer A (4×0.920 mL) with 266 μL (2×133 μL) of substrate solution).

At t=0, add 30 μL of working substrate solution with multi-channel pipette labeled “Briggs 303” and start the reading ([S]final: 5 μM).

Read with ex: 330 nm (20 nm) and em: 465 nm (20 nm) every 30 second for 10 minutes. The velocities are used to analyze and calculate the IC₅₀s.

Tables 4-6 show the activity of compounds when tested with the assay at 50, 500, 5000, 50000, and 500000 nM.

TABLE 4

| Compound | Concentration (nM) | % Inhibition |
|---|---|---|
| 1 | 500 | 65 |
| 2 | 50000 | 82 |
| 3 | 50000 | 96 |
| 4 | 50000 | 87 |
| 5 | 5000 | 71 |
| 6 | 5000 | 71 |
| 7 | 5000 | 75 |
| 8 | 5000 | 85 |
| 9 | 50000 | 72 |
| 10 | 5000 | 70 |
| 11 | 50000 | 80 |
| 12 | 50000 | 99 |
| 13 | 5000 | 86 |
| 14 | 50000 | 77 |
| 15 | 5000 | 86 |
| 16 | 50000 | 62 |
| 17 | 5000 | 64 |
| 18 | 5000 | 93 |
| 19 | 5000 | 99 |
| 20 | 5000 | 93 |
| 21 | 5000 | 93 |
| 22 | 5000 | 78 |
| 23 | 50000 | 93 |
| 24 | 5000 | 87 |
| 25 | 2000 | 23 |

TABLE 5

| Compound | Concentration (nM) | % Inhibition |
|---|---|---|
| 101 | 5000 | 95 |
| 102 | 50000 | 63 |
| 103 | 5000 | 87 |
| 104 | 50000 | 79 |
| 105 | 5000 | 99 |
| 106 | 500 | 82 |
| 107 | 5000 | 91 |
| 108 | 50000 | 80 |
| 109 | 500000 | 80 |
| 110 | 500 | 79 |
| 111 | 500 | 82 |
| 112 | 50000 | 80 |
| 113 | 5000 | 66 |
| 114 | 50000 | 74 |
| 115 | 500 | 89 |
| 116 | 5000 | 81 |
| 117 | 50000 | 81 |
| 118 | 5000 | 95 |
| 119 | 50000 | 98 |
| 120 | 5000 | 79 |
| 121 | 5000 | 91 |
| 122 | 500 | 88 |
| 123 | 5000 | 98 |
| 124 | 500 | 80 |
| 125 | 5000 | 98 |
| 126 | 5000 | 78 |
| 127 | 5000 | 84 |
| 128 | 500 | 89 |
| 129 | 50 | 70 |
| 130 | 5000 | 89 |
| 131 | 5000 | 98 |
| 132 | 50000 | 65 |
| 133 | 500 | 83 |
| 134 | 5000 | 83 |
| 135 | 5000 | 89 |
| 136 | 5000 | 83 |
| 137 | 5000 | 82 |
| 138 | 500 | 79 |
| 139 | 5000 | 70 |
| 140 | 500 | 78 |
| 141 | 5000 | 91 |
| 142 | 50000 | 84 |
| 143 | 5000 | 99 |
| 144 | 50000 | 66 |
| 145 | 5000 | 95 |
| 146 | 5000 | 73 |
| 147 | 5000 | 93 |
| 148 | 5000 | 76 |
| 149 | 50 | 95 |
| 150 | 5000 | 87 |
| 151 | 5000 | 90 |
| 152 | 5000 | 98 |
| 153 | 5000 | 92 |
| 154 | 5000 | 81 |
| 155 | | |
| 156 | 5000 | 79 |
| 157 | 2000 | 81 |

TABLE 6

| Compound | Concentration (nM) | % Inhibition |
|---|---|---|
| 158 | 500 | 72 |
| 159 | 5000 | 86 |
| 160 | 500 | 81 |
| 161 | 5000 | 90 |
| 162 | 500 | 78 |
| 163 | 5000 | 89 |
| 164 | 5000 | 59 |
| 165 | 5000 | 86 |

### FORMULATION EXAMPLES

The following are representative pharmaceutical formulations containing a compound of the present invention.

### Example 1

### Tablet Formulation

The following ingredients are mixed intimately and pressed into single scored tablets.

| Ingredient | Quantity per tablet, mg |
|---|---|
| Compound of the invention | 400 |
| Cornstarch | 50 |
| Croscarmellose sodium | 25 |
| Lactose | 120 |
| Magnesium stearate | 5 |

### Example 2

### Capsule Formulation

The following ingredients are mixed intimately and loaded into a hard-shell gelatin capsule.

| Ingredient | Quantity per tablet, mg |
|---|---|
| Compound of the invention | 200 |
| Lactose, spray-dried | 148 |
| Magnesium stearate | 2 |

### Example 3

### Suspension Formulation

The following ingredients are mixed to form a suspension for oral administration (q.s.=sufficient amount).

| Ingredient | Amount | |
|---|---|---|
| Compound of the invention | 1.0 | g |
| Fumaric acid | 0.5 | g |
| Sodium chloride | 2.0 | g |
| Methyl paraben | 0.15 | g |
| Propyl paraben | 0.05 | g |
| Granulated sugar | 25.0 | g |
| Sorbitol (70% solution) | 13.0 | g |
| Veegum K (Vanderbilt Co) | 1.0 | g |
| Flavoring | 0.035 | mL |
| colorings | 0.5 | mg |
| distilled water | q.s. to 100 | mL |

### Example 4

### Injectable Formulation

The following ingredients are mixed to form an injectable formulation.

| Ingredient | Quantity per tablet, mg |
|---|---|
| Compound of the invention | 0.2 mg-20 mg |
| sodium acetate buffer solution, 0.4 M | 2.0 mL |
| HCl (1N) or NaOH (1N) | q.s. to suitable pH |
| water (distilled, sterile) | q.s. to 20 mL |

### Example 5

### Suppository Formulation

A suppository of total weight 2.5 g is prepared by mixing the compound of the invention with Witepsol® H-15 (triglycerides of saturated vegetable fatty acid; Riches-Nelson, Inc., New York), and has the following composition:

| Ingredient | Quantity per tablet, mg |
|---|---|
| Compound of the invention | 500 mg |
| Witepsol ® H-15 | balance |

While the invention has been particularly shown and described with referenced to preferred embodiments, it will be understood by those skilled in the art that various changes in form and detail may be made without departing from the spirit and scope of the invention.

1. A compound or stereoisomer of Formula A or a pharmaceutically acceptable salt of the compound or the stereoisomer: 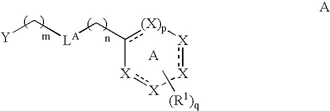
wherein
Y is selected from the group consisting of cycloalkyl, substituted cycloalkyl, phenyl, substituted phenyl, heterocyclyl, substituted heterocyclyl, heteroaryl, and substituted heteroaryl;
L^{A} is a linker of the formula: 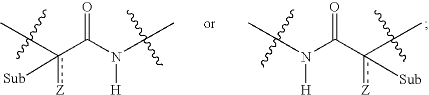
Sub is not present and Z is O if [symbol] connected thereto is a double bond, or Sub is hydrogen or alkyl, and Z is OH if [symbol] connected thereto is a single bond;
each X in ring A is independently selected from the group consisting of N, NH, NR¹, O, CH, CH₂, CHR¹, and CR¹R¹, with the proviso that at least two X's of the A ring are independently CH, CH₂, CHR¹, or CR¹R¹;
p is zero or one;
each R¹ is independently selected from the group consisting of alkyl, substituted alkyl, alkenyl, substituted alkenyl, alkynyl, substituted alkynyl, aryl, substituted aryl, heteroaryl, substituted heteroaryl, cycloalkyl, substituted cycloalkyl, heterocyclic, substituted heterocyclic, halo, hydroxy, nitro, cyano, alkoxy, substituted alkoxy, aryloxy, substituted aryloxy, acyl, aminocarbonyl, carboxy, carboxy ester, amino, substituted amino, acylamino, (carboxyl ester)amino, aminocarbonylamino, aminosulfonyl, substituted sulfonyl, and (substituted sulfonyl)amino;
q is 0, 1, 2, 3, or 4; [symbol]
of the ring A indicates a single or double bond;
m is 0, 1, 2, 3, 4, or 5; and
n is 0, 1, 2, 3, 4, or 5;
provided that the compound or pharmaceutically acceptable salt thereof is not
N-(1-benzylpiperidin-4-yl)-2-hydroxy-3-methyl-2-o-tolylbutanamide;
N-(1-benzylpiperidin-4-yl)-2-(3,5-difluorophenyl)-2-hydroxy-3-methylbutanamide;
N-(1-(4-fluorobenzyl)piperidin-4-yl)-2-hydroxy-3-methyl-2-phenylbutanamide;
N-(1-benzylpiperidin-4-yl)-2-hydroxy-3-methyl-2-(3-(trifluoromethyl)phenyl)butanamide;
N-(1-benzylpiperidin-4-yl)-2-hydroxy-3-methyl-2-m-tolylbutanamide;
N-(1-benzylpiperidin-4-yl)-3-ethyl-2-hydroxy-2-phenylpentanamide;
2-hydroxy-2-phenyl-N-(4-(piperidin-1-yl)phenyl)acetamide;
N-(2,4-dimethylphenyl)-2-oxo-2-phenylacetamide;
N1,N2-bis(2-(2-(4-nitrophenylamino)-2-oxoacetyl)phenyl)oxalamide;
2-(2,6-diphenoxyphenyl)-N-(6-methyl-2,4-bis(methylthio)pyridin-3-yl)-2-oxoacetamide;
2-(4-fluorophenyl)-N-(6-methyl-2,4-bis(methylthio)pyridin-3-yl)-2-oxoacetamide;
2-(4-bromophenyl)-N-(6-methyl-2,4-bis(methylthio)pyridin-3-yl)-2-oxoacetamide;
4-(2-(4-(dimethylamino)phenyl)-2-oxoacetamido)benzenesulfonic acid;
2-(2-acetamido-5-bromophenyl)-N-(4-ethoxyphenyl)-2-oxoacetamide;
N-(3-methoxy-4-(oxazol-5-yl)phenyl)-2-oxo-2-phenylacetamide;
2-(2-acetamidophenyl)-N-(4-ethylphenyl)-2-oxoacetamide;
2-(2-acetamidophenyl)-N-(3,5-dichlorophenyl)-2-oxoacetamide;
4-(2-(2-acetamidophenyl)-2-oxoacetamido)-N-phenethylbenzamide;
3-(2-(2-acetamidophenyl)-2-oxoacetamido)-N-(2,4,6-trifluorobenzyl)benzamide;
3-(2-(2-acetamidophenyl)-2-oxoacetamido)-N-(4-chlorophenethyl)benzamide;
2-oxo-N-(4-(4-oxo-4,5,6,7-tetrahydro-1H-pyrrolo[3,2-c]pyridin-2-yl)pyridin-2-yl)-2-phenylacetamide;
2-(2-acetamidophenyl)-N-(3,5-dimethylphenyl)-2-oxoacetamide;
2-(2-acetamidophenyl)-N-(4-(ethylthio)phenyl)-2-oxoacetamide;
2-(4-ethoxydibenzo[b,d]furan-1-yl)-2-oxo-N-(pyridin-4-yl)acetamide;
N-(2-(1-naphthamido)ethyl)-4-(2-(4-nitrophenyl)-2-oxoacetamido)piperidine-4-carboxamide;
2-oxo-N,2-di-p-tolylacetamide; or
3-methyl-2-(2-oxo-2-(p-tolylamino)acetyl)benzenesulfonic acid.

2. A compound or stereoisomer of claim 1 of Formula I or a pharmaceutically acceptable salt of the compound or the stereoisomer: 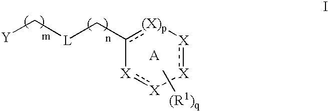
wherein
Y is selected from the group consisting of cycloalkyl, substituted cycloalkyl, phenyl, substituted phenyl, heterocyclyl, substituted heterocyclyl, heteroaryl, and substituted heteroaryl;
L is a linker of the formula: 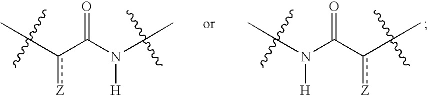
Z is O if [symbol] connected thereto is a double bond or Z is OH if [symbol] connected thereto is a single bond;
each X in ring A is independently selected from the group consisting of N, NH, NR¹, O, CH, CH₂, CHR¹, and CR¹R¹, with the proviso that at least two X's of the A ring are independently CH, CH₂, CHR¹, or CR¹R¹;
p is zero or one;
each R¹ is independently selected from the group consisting of alkyl, substituted alkyl, alkenyl, substituted alkenyl, alkynyl, substituted alkynyl, aryl, substituted aryl, heteroaryl, substituted heteroaryl, cycloalkyl, substituted cycloalkyl, heterocyclic, substituted heterocyclic, halo, hydroxy, nitro, cyano, alkoxy, substituted alkoxy, aryloxy, substituted aryloxy, acyl, aminocarbonyl, carboxy, carboxy ester, amino, substituted amino, acylamino, (carboxyl ester)amino, aminocarbonylamino, aminosulfonyl, substituted sulfonyl, and (substituted sulfonyl)amino;
q is 0, 1, 2, 3, or 4; [symbol]
of the ring A indicates a single or double bond;
m is 0, 1, 2, 3, 4, or 5; and
n is 0, 1, 2, 3, 4, or 5.

3. A compound of claim 2, wherein Y is cycloalkyl or substituted cycloalkyl.

4. A compound of claim 3, wherein Y is cyclohexyl, substituted cycloalkyl, adamantyl, or substituted adamantyl.

5. A compound of claim 4, wherein Y is selected from the group consisting of

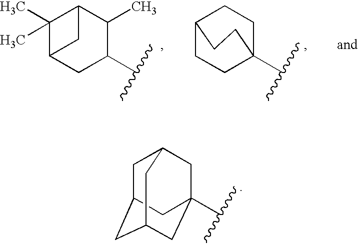

6. A compound of claim 3, wherein Y is spiro[4.5]dec-8-yl:

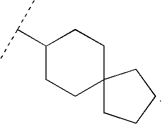

7. A compound of claim 2, wherein Y is C₆₋₁₀ heterocycloalkyl.

8. A compound of claim 7, wherein Y is quinuclidin-1-yl having the structure

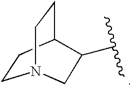

9. A compound of claim 2, wherein Y is selected from the group consisting of phenyl and substituted phenyl.

10. A compound of claim 2, wherein that at least four X's of the ring A are independently CH, CH₂, CHR¹, or CR¹R¹.

11. A compound of claim 2, wherein each [symbol] of the ring A is a double bond.

12. A compound of claim 2, wherein each [symbol] of the ring A is a single bond.

13. A compound of claim 2, wherein the ring A is selected from the group consisting of phenyl, pyridinyl, cyclohexyl, and piperidinyl.

14. A compound of claim 13, wherein the ring A is selected from the group consisting of phenyl, piperidinyl, and cyclohexyl.

15. A compound of claim 2, wherein each R¹ is independently selected from the group consisting of alkoxy, substituted alkoxy, aminocarbonyl, haloalkyl, heterocyclic, substituted sulfonyl, acyl, carboxy, carboxyl ester, amino, substituted amino, acylamino, (carboxyl ester)amino, aminosulfonyl, and (substituted sulfonyl)amino.

16. A compound of claim 2, wherein q is 1.

17. A compound of claim 16, wherein R¹ is in the 3-position or 4-position.

18. A compound of claim 16, wherein R¹ is selected from the group consisting of alkoxy, substituted alkoxy, acyl, carboxy, carboxyl ester, aminocarbonyl, amino, substituted amino, acylamino, (carboxyl ester)amino, aminocarbonylamino, aminosulfonyl, substituted sulfonyl, (substituted sulfonyl)amino, haloalkyl, and heterocyclic.

19. A compound of claim 2, wherein Z is O and [symbol] connected thereto is a double bond.

20. A compound of claim 2, wherein Z is OH and [symbol] connected thereto is a single bond.

21. A compound of claim 2, wherein m is 0, 1, or 2.

22. A compound of claim 2, wherein n is 0, 1, or 2.

23. A compound or stereoisomer of claim 2 of Formula Ia or a pharmaceutically acceptable salt of the compound or the stereoisomer: 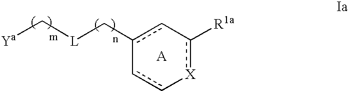
wherein
Y^{a} is C₆₋₁₀ cycloalkyl, substituted C₅₋₁₀ cycloalkyl, or 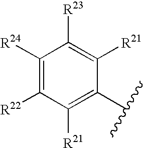
wherein each R²¹ is independently hydrogen or fluoro;
R²², R²³, and R²⁴ are independently selected from the group consisting of hydrogen, halo, alkyl, acyl, acyloxy, carboxyl ester, acylamino, aminocarbonyl, aminocarbonylamino, aminocarbonyloxy, aminosulfonylamino, (carboxyl ester)amino, aminosulfonyl, (substituted sulfonyl)amino, haloalkyl, haloalkoxy, haloalkylthio, cyano, and alkylsulfonyl;
L is a linker of the formula: 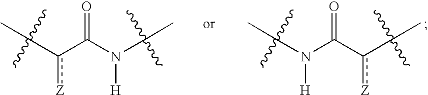
Z is O if [symbol] connected thereto is a double bond or Z is OH if [symbol] connected thereto is a single bond;
X in ring A is selected from the group consisting of N, NH, NR¹, O, CH, CH₂, CHR¹, and CR¹R¹;
each R¹ is independently selected from the group consisting of alkyl, substituted alkyl, alkenyl, substituted alkenyl, alkynyl, substituted alkynyl, aryl, substituted aryl, heteroaryl, substituted heteroaryl, cycloalkyl, substituted cycloalkyl, heterocyclic, substituted heterocyclic, halo, hydroxy, nitro, cyano, alkoxy, substituted alkoxy, aryloxy, substituted aryloxy, acyl, aminocarbonyl, carboxy, carboxy ester, amino, substituted amino, acylamino, (carboxyl ester)amino, aminocarbonylamino, aminosulfonyl, substituted sulfonyl, and (substituted sulfonyl)amino;
R^{1a} is hydrogen or R¹; [symbol]
of the ring A indicates a single or double bond;
m is 0, 1, 2, 3, 4, or 5; and
n is 0, 1, 2, 3, 4, or 5;
provided that the compound is not

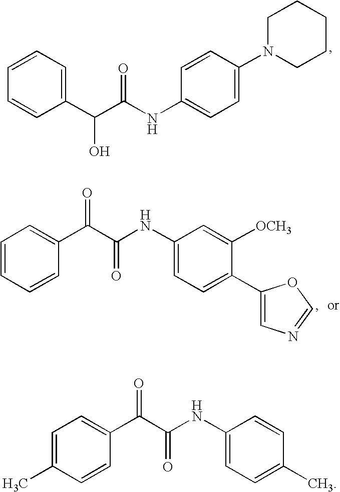

24. A compound or stereoisomer of claim 2 of Formula II or a pharmaceutically acceptable salt of the compound or the stereoisomer: 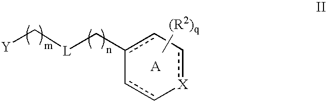
wherein
Y is selected from the group consisting of cyclohexyl, substituted cyclohexyl, adamantyl, substituted adamantyl, phenyl, substituted phenyl, heterocyclyl, and substituted heterocyclyl;
L is a linker of the formula: 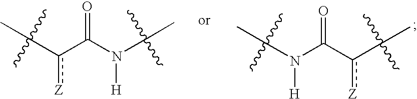
Z is O if [symbol] connected thereto is a double bond or Z is OH if [symbol] connected thereto is a single bond;
X in ring A is selected from the group consisting of N, NH, NR², O, CH, CH₂, CHR², and CR²R²;
each R² is independently selected from the group consisting of alkyl, substituted alkyl, alkenyl, substituted alkenyl, alkynyl, substituted alkynyl, aryl, substituted aryl, heteroaryl, substituted heteroaryl, cycloalkyl, substituted cycloalkyl, heterocyclic, substituted heterocyclic, halo, hydroxy, nitro, cyano, alkoxy, substituted alkoxy, aryloxy, substituted aryloxy, acyl, aminocarbonyl, carboxy, carboxy ester, amino, substituted amino, acylamino, (carboxyl ester)amino, aminocarbonylamino, aminosulfonyl, substituted sulfonyl, and (substituted sulfonyl)amino;
q is 0, 1, 2, 3, or 4; [symbol]
of the ring A indicates a single or double bond;
m is 0, 1, 2, 3, 4, or 5; and
n is 0, 1, 2, 3, 4, or 5;
provided that the compound or pharmaceutically acceptable salt thereof is not
2-hydroxy-2-phenyl-N-(4-(piperidin-1-yl)phenyl)acetamide;
N-(2,4-dimethylphenyl)-2-oxo-2-phenylacetamide;
N1,N2-bis(2-(2-(4-nitrophenylamino)-2-oxoacetyl)phenyl)oxalamide;
2-(2,6-diphenoxyphenyl)-N-(6-methyl-2,4-bis(methylthio)pyridin-3-yl)-2-oxoacetamide;
2-(4-fluorophenyl)-N-(6-methyl-2,4-bis(methylthio)pyridin-3-yl)-2-oxoacetamide;
2-(4-bromophenyl)-N-(6-methyl-2,4-bis(methylthio)pyridin-3-yl)-2-oxoacetamide;
4-(2-(4-(dimethylamino)phenyl)-2-oxoacetamido)benzenesulfonic acid;
2-(2-acetamido-5-bromophenyl)-N-(4-ethoxyphenyl)-2-oxoacetamide;
N-(3-methoxy-4-(oxazol-5-yl)phenyl)-2-oxo-2-phenylacetamide;
2-(2-acetamidophenyl)-N-(4-ethylphenyl)-2-oxoacetamide;
2-(2-acetamidophenyl)-N-(3,5-dichlorophenyl)-2-oxoacetamide;
4-(2-(2-acetamidophenyl)-2-oxoacetamido)-N-phenethylbenzamide;
3-(2-(2-acetamidophenyl)-2-oxoacetamido)-N-(2,4,6-trifluorobenzyl)benzamide;
3-(2-(2-acetamidophenyl)-2-oxoacetamido)-N-(4-chlorophenethyl)benzamide;
2-oxo-N-(4-(4-oxo-4,5,6,7-tetrahydro-1H-pyrrolo[3,2-c]pyridin-2-yl)pyridin-2-yl)-2-phenylacetamide;
2-(2-acetamidophenyl)-N-(3,5-dimethylphenyl)-2-oxoacetamide;
2-(2-acetamidophenyl)-N-(4-(ethylthio)phenyl)-2-oxoacetamide;
2-(4-ethoxydibenzo[b,d]furan-1-yl)-2-oxo-N-(pyridin-4-yl)acetamide;
N-(2-(1-naphthamido)ethyl)-4-(2-(4-nitrophenyl)-2-oxoacetamido)piperidine-4-carboxamide;
2-oxo-N,2-di-p-tolylacetamide; or
3-methyl-2-(2-oxo-2-(p-tolylamino)acetyl)benzenesulfonic acid.

25. A compound of claim 24, wherein Y is cyclohexyl, adamantyl, substituted cyclohexyl, substituted adamantyl, substituted phenyl, or substituted heterocyclyl, wherein the substituent on Y is selected from the group consisting of alkyl, halo, and haloalkyl.

26. A compound of claim 24, wherein each [symbol] of the ring A is a double bond.

27. A compound of claim 24, wherein each [symbol] of the ring A is a single bond.

28. A compound of claim 24, wherein the ring A is selected from the group consisting of phenyl, piperidinyl, and cyclohexyl.

29. A compound of claim 24, wherein each R² is independently selected from the group consisting of alkoxy, substituted alkoxy, aminocarbonyl, haloalkyl, heterocyclic, substituted sulfonyl, acyl, carboxy, carboxyl ester, amino, substituted amino, acylamino, (carboxyl ester)amino, aminosulfonyl, and (substituted sulfonyl)amino.

30. A compound of claim 24, wherein q is 1.

31. A compound of claim 30, wherein R is in the 3-position or 4-position.

32. A compound of claim 30, wherein R² is selected from the group consisting of alkoxy, substituted alkoxy, acyl, carboxy, carboxyl ester, aminocarbonyl, amino, substituted amino, acylamino, (carboxyl ester)amino, aminocarbonylamino, aminosulfonyl, substituted sulfonyl, (substituted sulfonyl)amino, haloalkyl, and heterocyclic.

33. A compound of claim 30, wherein Z is O and [symbol] connected thereto is a double bond.

34. A compound of claim 30, wherein Z is OH and [symbol] connected thereto is a single bond.

35. A compound of claim 30, wherein m is 0, 1, or 2.

36. A compound of claim 30, wherein n is 0, 1, or 2.

37. A compound or stereoisomer of claim 2 of Formula III or a pharmaceutically acceptable salt of the compound or the stereoisomer: 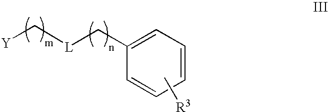
wherein
Y is selected from the group consisting of cyclohexyl, substituted cyclohexyl, adamantyl, substituted adamantyl, phenyl, substituted phenyl, heterocyclyl, and substituted heterocyclyl;
L is a linker of the formula: 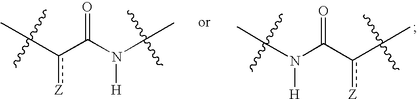
Z is O if [symbol] connected thereto is a double bond or Z is OH if [symbol] connected thereto is a single bond;
R³ is selected from the group consisting of alkyl, substituted alkyl, alkenyl, substituted alkenyl, alkynyl, substituted alkynyl, aryl, substituted aryl, heteroaryl, substituted heteroaryl, cycloalkyl, substituted cycloalkyl, heterocyclic, substituted heterocyclic, halo, hydroxy, nitro, cyano, alkoxy, substituted alkoxy, aryloxy, substituted aryloxy, acyl, aminocarbonyl, carboxy, carboxy ester, amino, substituted amino, acylamino, (carboxyl ester)amino, aminocarbonylamino, aminosulfonyl, substituted sulfonyl, and (substituted sulfonyl)amino;
m is 0, 1, or 2; and
n is 0, 1, or 2;
provided that the compound or pharmaceutically acceptable salt thereof is not
2-hydroxy-2-phenyl-N-(4-(piperidin-1-yl)phenyl)acetamide;
N-(2,4-dimethylphenyl)-2-oxo-2-phenylacetamide;
N1,N2-bis(2-(2-(4-nitrophenylamino)-2-oxoacetyl)phenyl)oxalamide;
2-(2,6-diphenoxyphenyl)-N-(6-methyl-2,4-bis(methylthio)pyridin-3-yl)-2-oxoacetamide;
2-(4-fluorophenyl)-N-(6-methyl-2,4-bis(methylthio)pyridin-3-yl)-2-oxoacetamide;
2-(4-bromophenyl)-N-(6-methyl-2,4-bis(methylthio)pyridin-3-yl)-2-oxoacetamide;
4-(2-(4-(dimethylamino)phenyl)-2-oxoacetamido)benzenesulfonic acid;
2-(2-acetamido-5-bromophenyl)-N-(4-ethoxyphenyl)-2-oxoacetamide;
N-(3-methoxy-4-(oxazol-5-yl)phenyl)-2-oxo-2-phenylacetamide;
2-(2-acetamidophenyl)-N-(4-ethylphenyl)-2-oxoacetamide;
2-(2-acetamidophenyl)-N-(3,5-dichlorophenyl)-2-oxoacetamide;
4-(2-(2-acetamidophenyl)-2-oxoacetamido)-N-phenethylbenzamide;
3-(2-(2-acetamidophenyl)-2-oxoacetamido)-N-(2,4,6-trifluorobenzyl)benzamide;
3-(2-(2-acetamidophenyl)-2-oxoacetamido)-N-(4-chlorophenethyl)benzamide;
2-oxo-N-(4-(4-oxo-4,5,6,7-tetrahydro-1H-pyrrolo[3,2-c]pyridin-2-yl)pyridin-2-yl)-2-phenylacetamide;
2-(2-acetamidophenyl)-N-(3,5-dimethylphenyl)-2-oxoacetamide;
2-(2-acetamidophenyl)-N-(4-(ethylthio)phenyl)-2-oxoacetamide;
2-(4-ethoxydibenzo[b,d]furan-1-yl)-2-oxo-N-(pyridin-4-yl)acetamide;
N-(2-(1-naphthamido)ethyl)-4-(2-(4-nitrophenyl)-2-oxoacetamido)piperidine-4-carboxamide;
2-oxo-N,2-di-p-tolylacetamide; or
3-methyl-2-(2-oxo-2-(p-tolylamino)acetyl)benzenesulfonic acid.

38. A compound of claim 37, wherein Y is cyclohexyl, adamantyl, substituted cyclohexyl, substituted adamantyl, substituted phenyl, heterocyclyl, or substituted heterocyclyl, where the substituent on Y is selected from the group consisting of alkyl, halo, and haloalkyl.

39. A compound of claim 37, wherein each R³ is independently selected from the group consisting of alkoxy, substituted alkoxy, aminocarbonyl, haloalkyl, heterocyclic, substituted sulfonyl, acyl, carboxy, carboxyl ester, amino, substituted amino, acylamino, (carboxyl ester)amino, aminosulfonyl, and (substituted sulfonyl)amino.

40. A compound of claim 37, wherein Z is O and [symbol] connected thereto is a double bond.

41. A compound of claim 37, wherein Z is OH and [symbol] connected thereto is a single bond.

42. A compound or stereoisomer of claim 2 of Formula IV or a pharmaceutically acceptable salt of the compound or the stereoisomer: 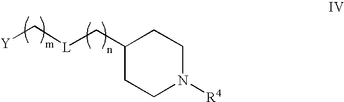
wherein
Y is selected from the group consisting of cyclohexyl, substituted cyclohexyl, adamantyl, substituted adamantyl, phenyl, substituted phenyl, heterocyclyl, and substituted heterocyclyl;
L is a linker of the formula: 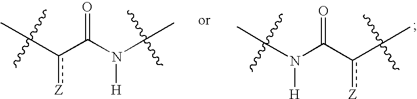
Z is O if [symbol] connected thereto is a double bond or Z is OH if [symbol] connected thereto is a single bond;
R⁴ is selected from the group consisting of alkyl, substituted alkyl, alkenyl, substituted alkenyl, alkynyl, substituted alkynyl, aryl, substituted aryl, heteroaryl, substituted heteroaryl, cycloalkyl, substituted cycloalkyl, heterocyclic, substituted heterocyclic, cyano, acyl, aminocarbonyl, aminosulfonyl, substituted sulfonyl, (substituted sulfonyl)amino, and carboxy ester;
m is 0, 1, or 2; and
n is 0, 1, or 2.

43. A compound of claim 42, wherein Y is cyclohexyl, adamantyl, substituted cyclohexyl, substituted adamantyl, substituted phenyl, or substituted heterocyclyl, where the substituent on Y is selected from the group consisting of alkyl, halo, and haloalkyl.

44. A compound of claim 42, wherein R⁴ is selected from the group consisting of substituted sulfonyl and acyl.

45. A compound of claim 42, wherein Z is O and [symbol] connected thereto is a double bond.

46. A compound of claim 43, wherein Z is OH and [symbol] connected thereto is a single bond.

47. A compound of claim 2 or stereoisomer or pharmaceutically acceptable salt of the compound or the stereoisomer, wherein the compound is

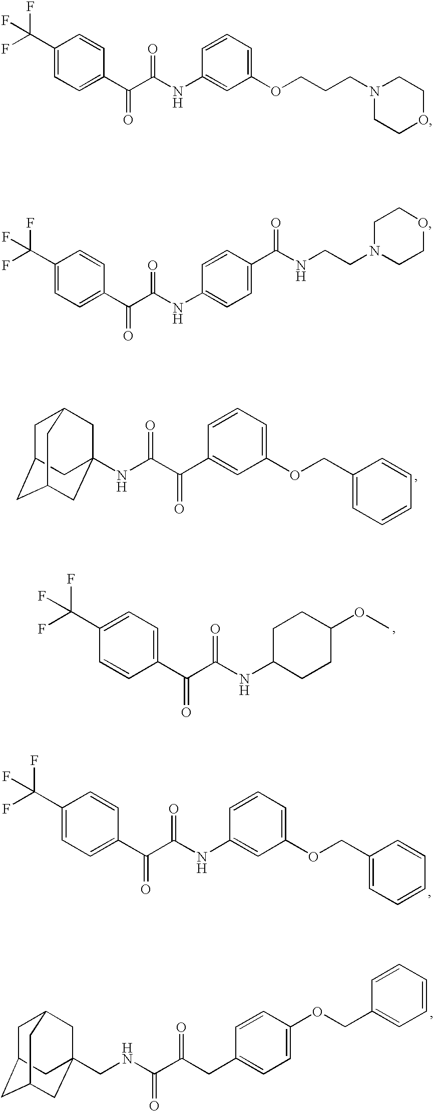

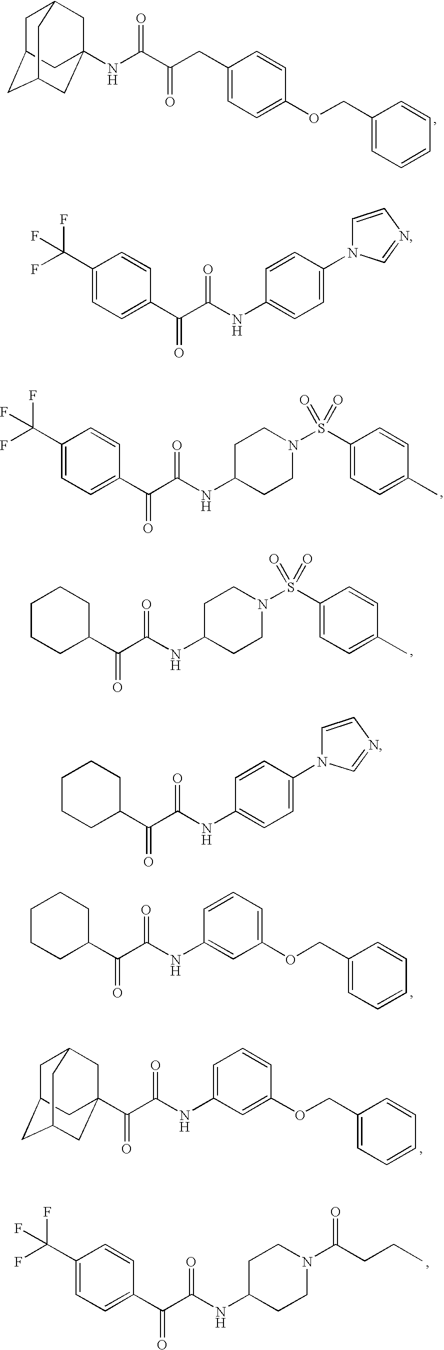

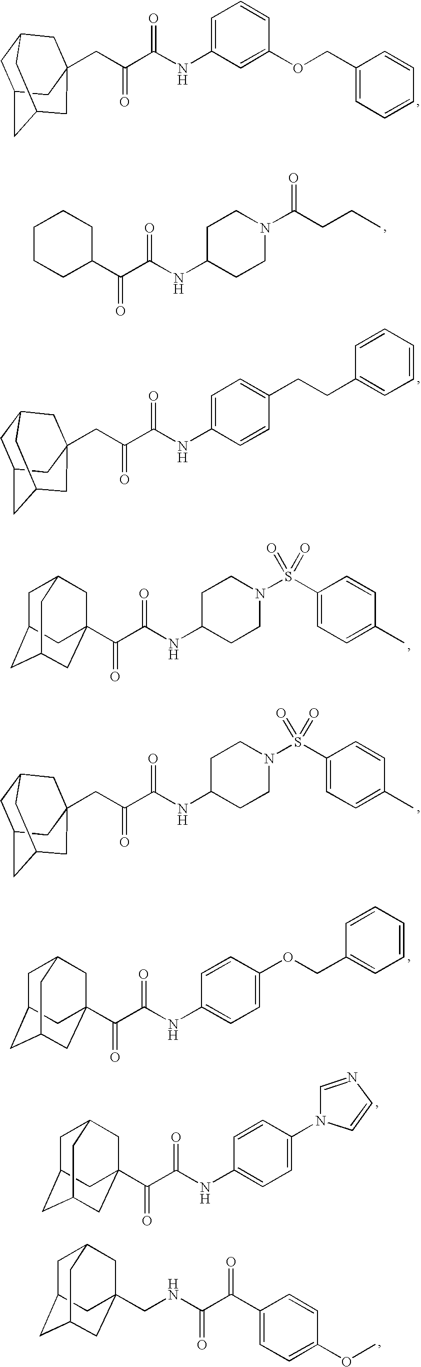

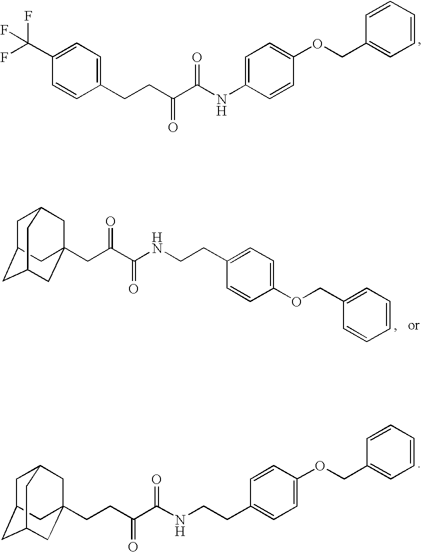

48. A compound of claim 2 or stereoisomer or pharmaceutically acceptable salt of the compound or the stereoisomer, wherein the compound is

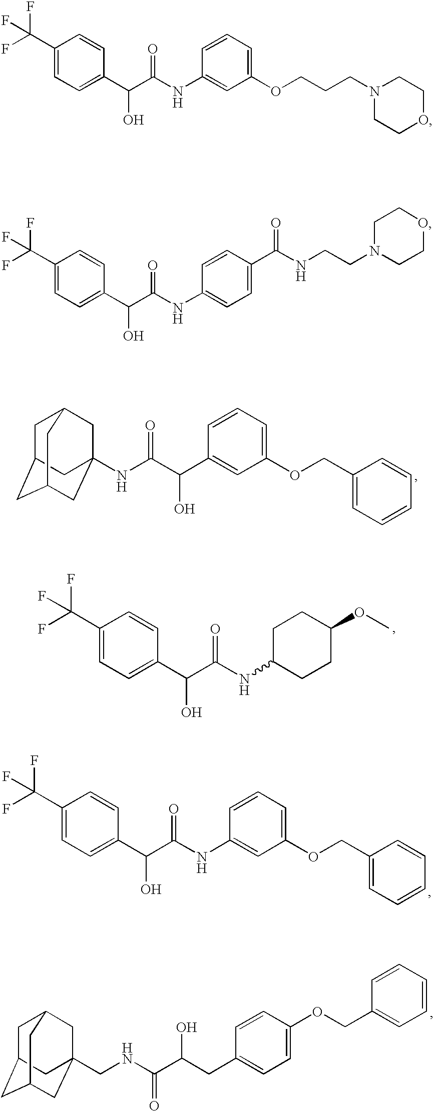

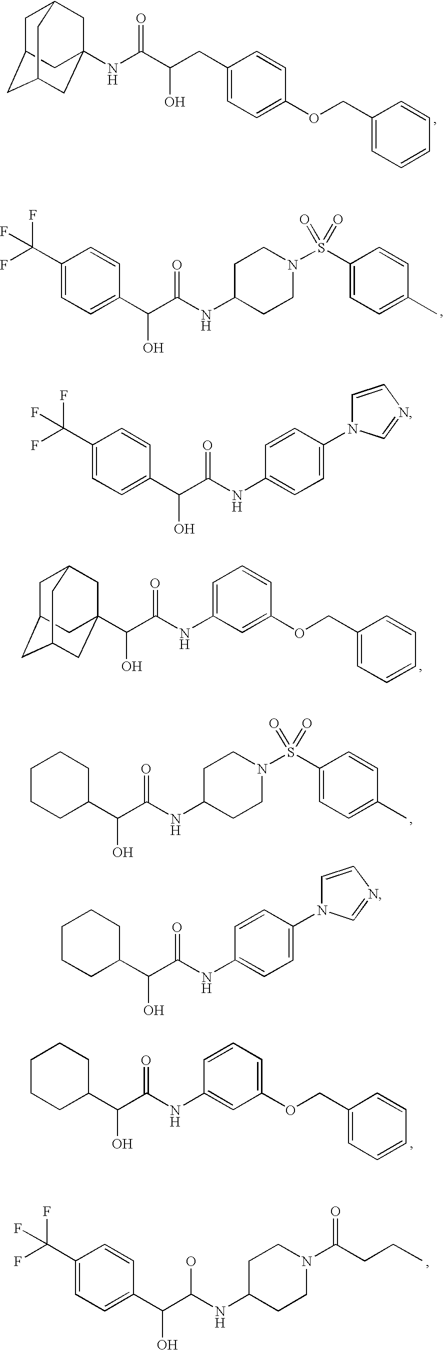

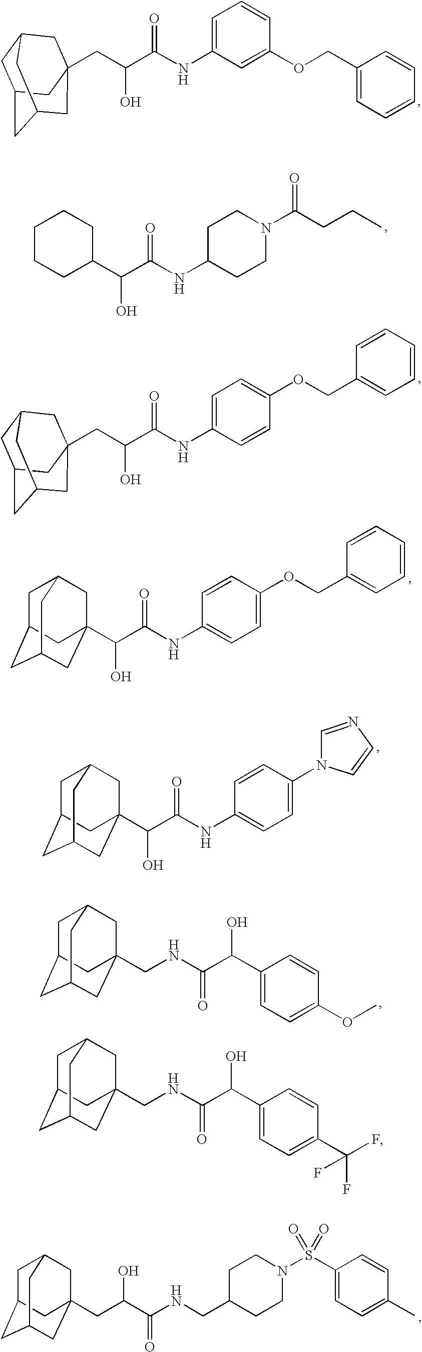

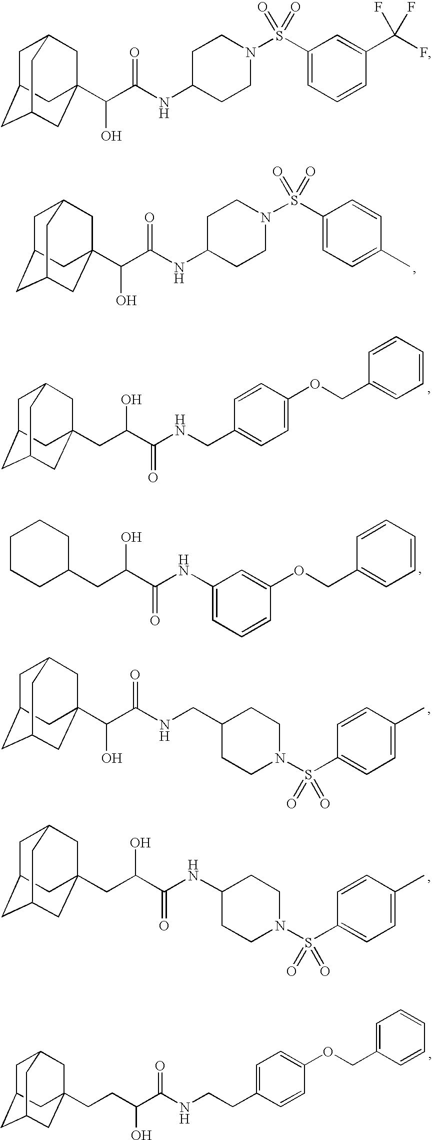

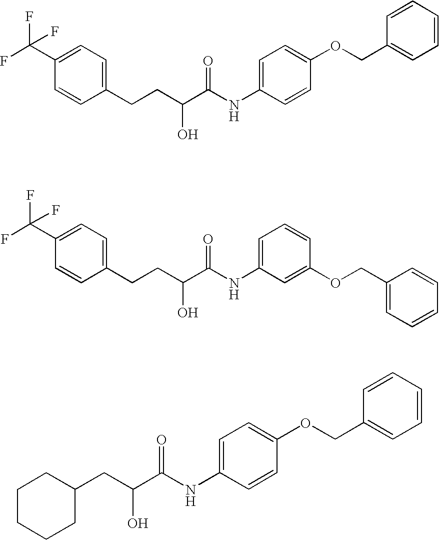

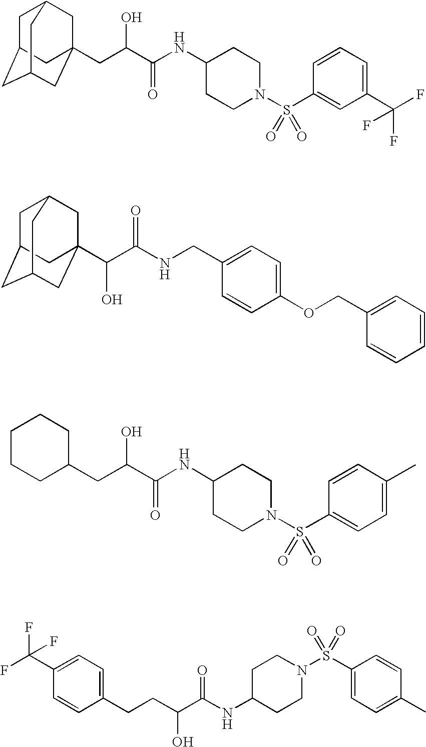

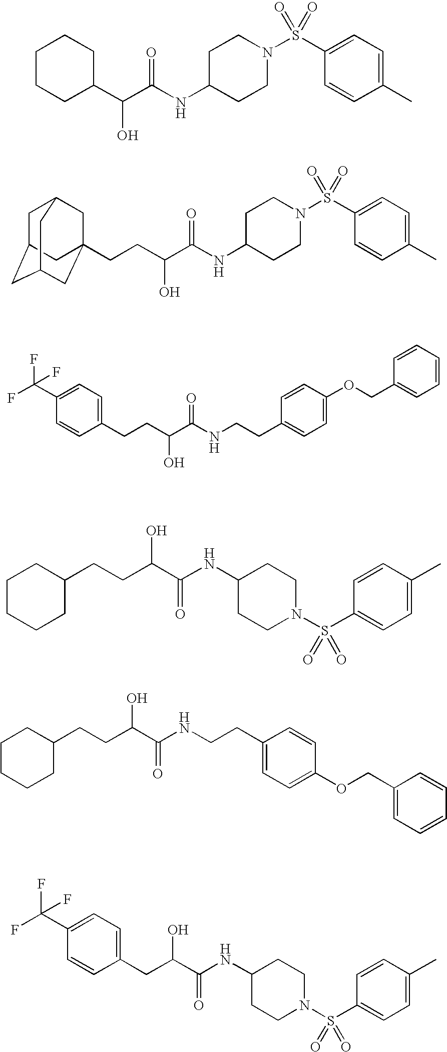

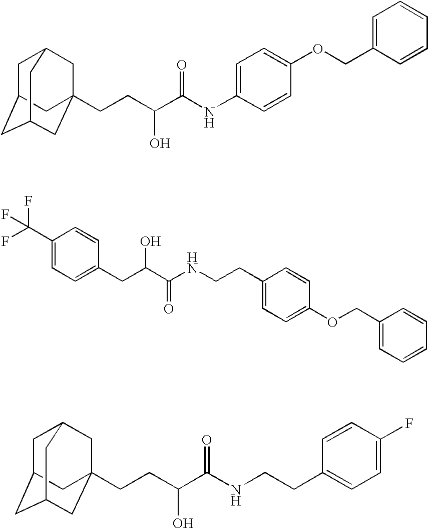

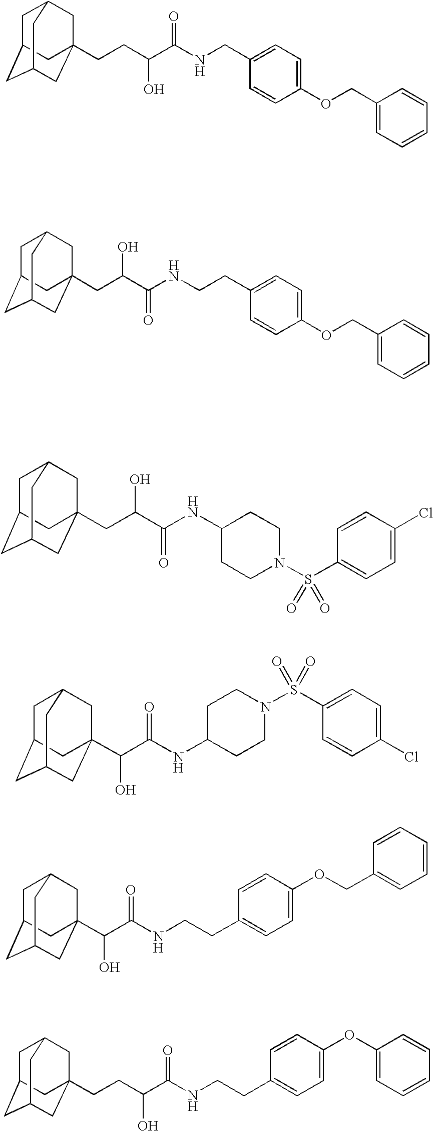

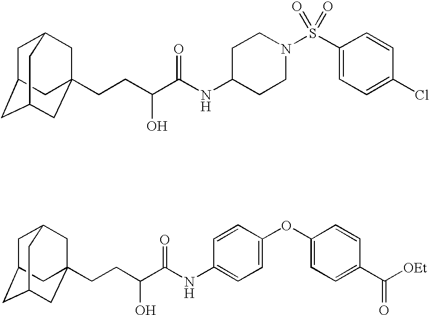

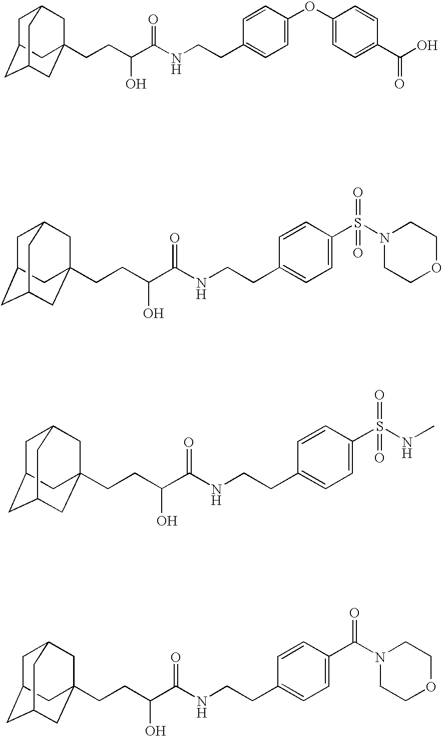

49. A compound or stereoisomer of Formula V or a pharmaceutically acceptable salt of the compound or the stereoisomer: 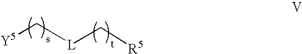
wherein
Y⁵ is selected from the group consisting of cycloalkyl, substituted cycloalkyl, phenyl, substituted phenyl, heterocyclyl, substituted heterocyclyl, heteroaryl, and substituted heteroaryl;
L is a linker of the formula: 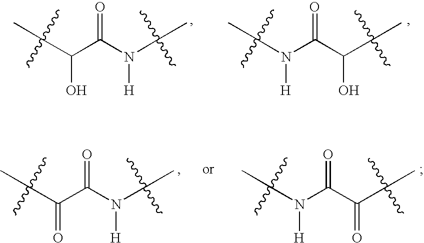
R⁵ is selected from the group consisting of alkyl, alkenyl, alkynyl, hydroxy, nitro, cyano, alkoxy, substituted alkoxy, aryloxy, substituted aryloxy, acyl, aminocarbonyl, carboxy, carboxy ester, amino, substituted amino, acylamino, (carboxyl ester)amino, aminocarbonylamino, aminosulfonyl, substituted sulfonyl, (substituted sulfonyl)amino, phenyl, substituted phenyl, heteroaryl, and substituted heteroaryl;
s is 0, 1, 2, 3, 4, or 5; and
t is 0, 1, 2, 3, 4, 5, 6, 7, 8, 9, 10, 11, 12, or 13.

50. A compound of claim 49, wherein Y⁵ is cycloalkyl or substituted cycloalkyl.

51. A compound of claim 50, wherein Y⁵ is cyclohexyl, adamantyl, substituted cyclohexyl, substituted adamantyl, substituted phenyl, or substituted heterocyclyl, wherein the substituent on Y is selected from the group consisting of alkyl, halo, and haloalkyl.

52. A compound of claim 49, wherein Y⁵ is phenyl or substituted phenyl.

53. A compound of claim 49, wherein s is 0, 1, 2, or 3 and t is 9, 10, 11, or 12.

54. A compound of claim 49, wherein R⁵ is carboxy or carboxy ester.

55. A compound of claim 49, wherein L is

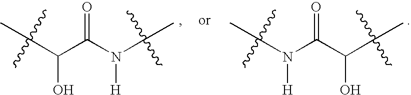

56. A compound of claim 49, wherein L is

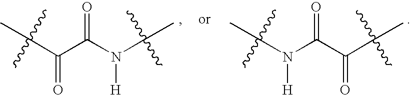

57. A compound of claim 49 or stereoisomer, or pharmaceutically acceptable salt of the compound or the stereoisomer, wherein the compound is

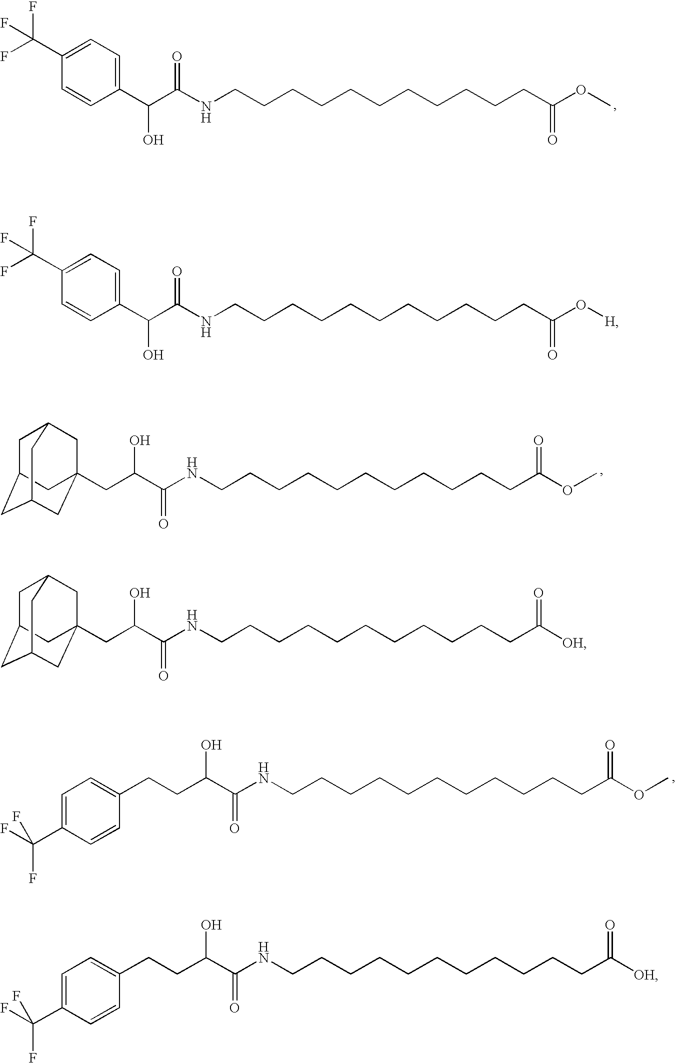

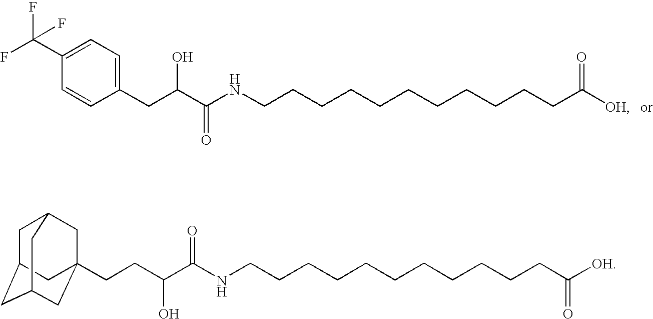

58. A pharmaceutical composition comprising a pharmaceutically acceptable carrier and a therapeutically effective amount of a compound or stereoisomer or pharmaceutically acceptable salt of the compound or the stereoisomer of claim 1 or claim 49 for treating a soluble epoxide hydrolase mediated disease.

59. (canceled)

60. A method for treating a soluble epoxide hydrolase mediated disease, said method comprising administering to a patient a pharmaceutical composition comprising a pharmaceutically acceptable carrier and a therapeutically effective amount of a compound or stereoisomer of claim 1.

61. A method for treating a soluble epoxide hydrolase mediated disease, said method comprising administering to a patient a pharmaceutical composition comprising a pharmaceutically acceptable carrier and a therapeutically effective amount of a compound or stereoisomer of Formula I or a pharmaceutically acceptable salt of the compound or the stereoisomer: 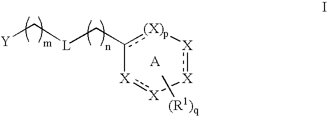
wherein
Y is selected from the group consisting of cycloalkyl, substituted cycloalkyl, phenyl, substituted phenyl, heterocyclyl, substituted heterocyclyl, heteroaryl, and substituted heteroaryl;
L is a linker of the formula: 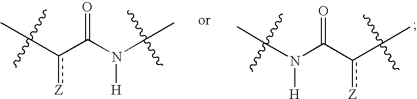
Z is O if [symbol] connected thereto is a double bond or Z is OH if [symbol] connected thereto is a single bond;
each X in ring A is independently selected from the group consisting of N, NH, NR¹, O, CH, CH₂, CHR¹, and CR¹R¹, with the proviso that at least two X's of the A ring are independently CH, CH₂, CHR¹, or CR¹R¹;
p is zero or one;
each R¹ is independently selected from the group consisting of alkyl, substituted alkyl, alkenyl, substituted alkenyl, alkynyl, substituted alkynyl, aryl, substituted aryl, heteroaryl, substituted heteroaryl, cycloalkyl, substituted cycloalkyl, heterocyclic, substituted heterocyclic, halo, hydroxy, nitro, cyano, alkoxy, substituted alkoxy, aryloxy, substituted aryloxy, acyl, aminocarbonyl, carboxy, carboxy ester, amino, substituted amino, acylamino, (carboxyl ester)amino, aminocarbonylamino, aminosulfonyl, substituted sulfonyl, and (substituted sulfonyl)amino;
q is 0, 1, 2, 3, or 4; [symbol]
of the ring A indicates a single or double bond;
m is 0, 1, 2, 3, 4, or 5; and
n is 0, 1, 2, 3, 4, or 5.

62. A method for treating a soluble epoxide hydrolase mediated disease, said method comprising administering to a patient a pharmaceutical composition comprising a pharmaceutically acceptable carrier and a therapeutically effective amount of a compound or stereoisomer of Formula V or a pharmaceutically acceptable salt of the compound or the stereoisomer: 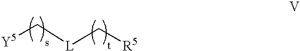
wherein
Y⁵ is selected from the group consisting of cycloalkyl, substituted cycloalkyl, phenyl, substituted phenyl, heterocyclyl, substituted heterocyclyl, heteroaryl, and substituted heteroaryl;
L is a linker of the formula: 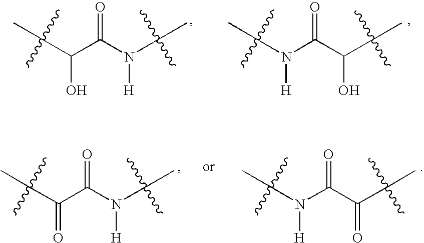
R⁵ is selected from the group consisting of alkyl, alkenyl, alkynyl, hydroxy, nitro, cyano, alkoxy, substituted alkoxy, aryloxy, substituted aryloxy, acyl, aminocarbonyl, carboxy, carboxy ester, amino, substituted amino, acylamino, (carboxyl ester)amino, aminocarbonylamino, aminosulfonyl, substituted sulfonyl, (substituted sulfonyl)amino, phenyl, substituted phenyl, heteroaryl, and substituted heteroaryl;
s is 0, 1, 2, 3, 4, or 5; and
t is 0, 1, 2, 3, 4, 5, 6, 7, 8, 9, 10, 11, 12, or 13.
